SEQUENCE LISTING (8 sequences)

SEQ ID NO: 1 - DNA, 184 bp, Mus sp.
  source 1..184: mol_type = genomic DNA
tggcctcgttgttccacaaaacagcaagcgcgtcatttagcgtgtagtctgttgagatccagggctgtagatagcgcgtgcgtggaggcgcgaaagcttctgatgaggcgatctcaaagcttggtggagagctctttgtcaccgcacttcacaggacacctgagttgtacatgaggtcatacag

SEQ ID NO: 2 - DNA, 15 bp, synthetic construct
  source 1..15: mol_type = other DNA
gatagcgcgtgcgtg

SEQ ID NO: 3 - DNA, 11 bp, synthetic construct
  source 1..11: mol_type = other DNA
cgcgtgcgtgg

SEQ ID NO: 4 - DNA, 10 bp, synthetic construct
  source 1..10: mol_type = other DNA
cgcacgcgct

SEQ ID NO: 5 - DNA, 13 bp, synthetic construct
  source 1..13: mol_type = other DNA
aacagcaagcgcg

SEQ ID NO: 6 - DNA, 10 bp, synthetic construct
  source 1..10: mol_type = other DNA
tatgacctca

SEQ ID NO: 7 - DNA, 20 bp, synthetic construct
  source 1..20: mol_type = other DNA
wwcaactcaggtgtcctgtg

SEQ ID NO: 8 - DNA, 20 bp, synthetic construct
  source 1..20: mol_type = other DNA
wwcttctcaggtgtcctgtg

(19) United States
(12) Patent Application Publication
(10) Pub. No.: US 2026/0345659 A1
(43) Pub. Date: Nov. 5, 2026

(54) METHODS AND COMPOSITIONS FOR IMPROVING PANCREATIC BETA CELL FUNCTION AND GLUCOSE HOMEOSTASIS

(71) Applicant: President and Fellows of Harvard College, Cambridge, MA (US)

(72) Inventors: Nora Kory, Boston, MA (US); Byungsoo Kong, Boston, MA (US)

(21) Appl. No.: 19/464,862

(22) Filed: Jan. 30, 2026

Related U.S. Application Data
(60) Provisional application No. 63/751,915, filed on Jan. 31, 2025.

(51) Int. Cl. A61K 31/7076; A61P 3/10; C12Q 1/6876; G01N 33/573; G01N 33/68

(52) U.S. Cl. CPC A61K 31/7076; A61P 3/10; C12Q 1/6876; G01N 33/5735; G01N 33/68; C12Q 2600/158

(57) ABSTRACT

The present invention provides methods for detecting the presence of cellular senescence in a subject, the methods comprising detecting the level of biomarkers solute carrier family 25 member 51 (SLC25A51) and/or nicotinamide adenine dinucleotide (NAD+). The present invention provides methods and composition for improving pancreatic β-cell function, and for treating a metabolic disorder associated with cellular senescence (e.g., diabetes) in a subject in need thereof by inhibiting SLC25A51 expression or activity.

### RELATED APPLICATIONS

This application claims the benefit of priority to U.S. Provisional Application No. 63/751,915, filed on Jan. 31, 2025, the entire contents of which are incorporated herein by reference.

### GOVERNMENT SUPPORT

This invention was made with government support under GM151097 awarded by National Institutes of Health (NIH). The government has certain rights in this invention.

### SEQUENCE LISTING

The instant application contains a Sequence Listing which has been submitted electronically in XML file format and is hereby incorporated by reference in its entirety. Said XML copy, created on Apr. 22, 2026, is named 117823-38202_SL.xml and is 8,237 bytes in size.

### BACKGROUND OF THE INVENTION

Aging is a complex phenomenon marked by changes in biological processes and metabolism, and increasing disease susceptibility. The redox cofactor nicotinamide adenine dinucleotide (NAD+) links metabolic control with cellular and epigenetic states. Disrupted NAD+ levels or redox balance are observed in aging and age-related diseases across model organisms and tissues. Altered NAD+ levels and NAD+/NADH redox balance are also linked to mitochondrial dysfunction and cellular senescence, which are key hallmarks of aging (Yoshino et al, Cell metabolism 27.3 (2018): 513-528; Lopez-Otin et al, Cell (2018) 186, 243-278).

Senescent cells are characterized by irreversible cell cycle arrest in response to aging-related stress. Likewise, the phenotype of senescent cells varies depending on the physiological context.

Senescence protects against tumorigenesis by stably arresting mitotic cells, such as fibroblasts or immune cells, but it also contributes to changes in metabolic activities and gene expression associated with inflammation and aging. Postmitotic cells, which are unreplaceable or infrequently replaced with age, including pancreatic beta cells, neurons and oocytes, are particularly vulnerable to metabolic stress and can also become senescent.

Cellular NAD+ levels and NAD+/NADH ratios are altered in beta-cell aging and diabetes.

Whole-body knockout of nicotinamide riboside kinase 1 (NRK1) in mice reduced pancreatic NAD+ levels, impaired glucose tolerance, and reduced insulin secretion and sensitivity upon a high-fat diet (Cercillieux et al, Mol Metab (2022) 66, 101605). Similarly, nicotinamide mononucleotide adenylyl transferase 2 (NMNAT2), a cytosolic NAD+ synthesis enzyme, was found to be induced in pancreatic beta cells in aged and diabetic mouse models, possibly as an adaptive response to increased glycolytic flux, compensating for decreased NADH re-oxidation. Conversely, NMNAT2 suppression improved beta-cell identity and function under increased glycolytic flux, potentially by altering NAD+ availability for nuclear NAD+ synthesis and its dependent enzymes, Sirt1 and PARP1, in a mechanism analogous to changes in compartmentalized NAD+ synthesis during adipogenesis. Overall, these findings highlight the critical role of NAD+ homeostasis and compartmentalization in regulating beta-cell function and the diabetes pathogenesis. However, the mechanisms by which compartmentalized NAD+ pools are established within cells and how they affect NAD+-dependent processes in relation to aging, cell state, function, and metabolism remain unclear.

Recently, solute carrier family 25 member 51 (SLC25A51) was identified as the mitochondrial NAD+ transporter in metazoans, which plays a critical role in regulating mitochondrial NAD+ levels and intracellular NAD+ compartmentalization. Through its role in shifting cellular NAD+ compartmentalization, SLC25A51 has also been shown to alter NAD+-dependent metabolic pathways, DNA repair, and cancer cell proliferation by uncoupling the mitochondrial NAD+/NADH ratio.

While it is well-established that NAD+ levels decline during aging and in age-related diseases, the processes contributing to this decline across different tissues, as well as the effects of altered NAD+ levels on cell function, metabolism and state are poorly understood. In particular, the molecular mechanisms underlying pancreatic beta-cell senescence remain unknown. Pancreatic beta-cell senescence is of particular interest as it is increasingly recognized as a contributor to the pathogenesis of both type 1 (T1D) and type 2 diabetes (T2D). This has highlighted the targeting of senescent beta cells as a promising therapeutic approach for diabetes.

Thus, there is a need in the art for the identification of specific markers to differentiate senescent from non-senescent beta-cell subsets. Further, there is a need for therapeutic approaches targeting pancreatic beta-cell senescence for the development of novel diabetes treatments.

### SUMMARY OF THE INVENTION

The present invention is based, at least in part, on the surprising discovery that the SLC25A51 gene is upregulated in the early and mid-stages of beta-cell senescence, and serves as a differentially expressed gene (DEG) marking the mid-stage. This upregulation of SLC25A51 was found to be associated with a loss of beta-cell identity, progression into senescence, and a decline in NAD+ levels and the NAD+/NADH ratio. Overexpression of SLC25A51 resulted in an increased mitochondrial NAD+/NADH ratio and induction of senescence and DNA damage markers (e.g., p16^{INK4A} p21^{Cip1/Waf1}, γ-H2AX, and NAMPT).

Further, Applicant surprisingly discovered that knockdown of SLC25A51 prevented the induction of senescence markers in vitro. Importantly, deleting SLC25A51 in vivo was found to improve glucose homeostasis and prevented insulin resistance by reducing circulating insulin and glucose levels in a mouse model of type 2 diabetes. Thus, SLC25A51 modulates senescence in pancreatic beta cells and acts as a factor linking NAD+ compartmentalization to the control of beta-cell state and function.

Accordingly, in one aspect, the present invention provides a method for detecting the presence of cellular senescence in a subject, the method comprising: (a) detecting the level of one or more biomarkers comprising solute carrier family 25 member 51 (SLC25A51) or nicotinamide adenine dinucleotide (NAD+) in a biological sample from the subject; and (b) comparing the level of the one or more biomarkers with a control sample, wherein an increase in the level of SLC25A51 in the subject as compared to the control sample indicates the presence of cellular senescence in the subject; and/or wherein a decrease in the level of NAD+ in the subject as compared to the control sample indicates the presence of cellular senescence in the subject.

In some embodiments, the level of SLC25A51 is a level of the SLC25A51 mRNA or SLC25A51 protein.

In some embodiments, the method further comprises determining the NAD+/NADH ratio in the sample from the subject, wherein a decrease in the NAD+/NADH ratio as compared to the control sample indicates the presence of cellular senescence in the subject.

In some embodiments, the method further comprises detecting the level of one or more molecules selected from the group consisting of P16INK4A, P21Cip1/Waf1, γ-H2AX, NAMPT, glutamine, glutamate, cellular ammonia and serum secretory associated phenotype (SASP) factors.

In some embodiments, the control sample is an earlier-in-time biological sample obtained from the subject, or wherein the control sample is a biological sample obtained from a younger subject.

In some embodiments, the biological sample is obtained from blood, testis, breast, pancreas, or liver tissue of the subject. In some embodiments, the biological sample is obtained from pancreas tissue and comprises pancreatic islet cells. In some embodiments, the pancreatic islet cells comprise pancreatic β-cells.

In another aspect, the present invention provides a method of improving pancreatic β-cell function in a subject in need thereof, the method comprising administering to the subject a therapeutically effective amount of an SLC25A51 inhibitor, thereby improving pancreatic β-cell function in the subject.

In some embodiments, administration of the SLC25A51 inhibitor increases glucose clearance, increases insulin sensitivity, and/or decreases body weight in the subject compared to a control. In some embodiments, glucose clearance is measured by a glucose tolerance test and insulin sensitivity is measured by an insulin tolerance test. In some embodiments, the blood glucose level in the subject after administration of the SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control. In some embodiments, the circulating insulin level in the subject after administration of SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control.

In some embodiments, the body weight of the subject after administration of the SLC25A51 inhibitor is at least 1.1 fold, at least 1.5 fold, at least 2 fold lower compared to the control.

In some embodiments, administration of the SLC25A51 inhibitor delays the onset of senescence in pancreatic β-cells of the subject. In some embodiments, the expression levels of one or more genes selected from the group consisting of CDKN2A, CDKN1A, CXCL10, IL-1β, and IL-6 in a biological sample from the subject is at least 1.1 fold, at least 1.5 fold, at least 2 fold, at least 5 fold, at least 10 fold, at least 20 fold, at least 50 fold, at least 100 fold, at least 200 fold lower compared to a control. In some embodiments, the levels of p16INK4A-positive senescent beta cells in the subject is at least 1.1 fold, at least 1.5 fold, at least 2 fold, at least 5 fold, at least 10 fold, at least 20 fold, at least 50 fold, at least 100 fold, at least 200 fold lower compared to a control.

In some embodiments, the control is measured prior to administration of the SLC25A51 inhibitor to the subject.

In some embodiments, the SLC25A51 inhibitor is selected from the group consisting of a small molecule, an antibody, an antisense oligonucleotide, an siRNA, an shRNA, and a miRNA. In some embodiments, the SLC25A51 inhibitor is fludarabine phosphate.

In some embodiments, the subject has diabetes. In some embodiments, the subject is a human subject.

In another aspect, the present invention provides a method of treating a metabolic disorder in a subject, the method comprising administering to the subject a therapeutically effective amount of an SLC25A51 inhibitor, thereby treating the metabolic disorder in the subject.

In some embodiments, the metabolic disorder is associated with cellular senescence and is selected from the group consisting of diabetes, metabolic syndrome (MetS), metabolic cardiovascular diseases, osteoporosis, atherosclerosis, hepatic steatosis, osteoarthritis, idiopathic pulmonary fibrosis, obesity-induced anxiety, tau-mediated neurodegenerative disease and chronic kidney disease.

In some embodiments, the SLC25A51 inhibitor is selected from the group consisting of a small molecule, an antibody, an antisense oligonucleotide, an siRNA, an shRNA, and a miRNA. In some embodiments, the SLC25A51 inhibitor is fludarabine phosphate.

In some embodiments, the subject has diabetes. In some embodiments, the subject is a human subject.

### BRIEF DESCRIPTION OF THE DRAWINGS

FIG. 1 shows that induction of mitochondrial NAD⁺ transporters is an evolutionarily conserved feature of chronological aging.

FIG. 1A: Analysis of published datasets for mRNA levels of SLC25A51 orthologs (Ndt1, Ndt2) in A. thaliana (GSE43616) and S. cerevisiae (GSE210032).

FIG. 1B: Levels of SLC25A51 and other mitochondrial proteins, as well as β-actin, were analyzed in various tissues harvested from 90-week-old C57BL/6N male and female mice (gonad, spleen, kidney, pancreatic islet cells, liver, and fat [preadipocytes]).

FIG. 1C: Levels of SLC25A51 and other mitochondrial proteins were analyzed in pancreatic islets and livers from 12-, 30-, 60-, 90-, and 120-week-old C57BL/6N male and female mice (n=2~3/group).

FIG. 1D: Levels of SLC25A51 protein, senescence markers (p16^{INK4a}, P21^{cip1/waf1}, p53, γ-H2AX, NAMPT), mitochondrial markers (Tom20, Citrate synthase [CS]), Cyp8b1 (hepatocyte marker), insulin (beta-cell marker), and CD3e (T-cell marker) were analyzed in tissue-derived primary cells (splenocytes, hepatocytes, pancreatic islet cells) isolated from 12- and 60-week-old male C57BL/6N mice (n=2/group).

(FIG. 1E-1J): Twelve-, 30-, and 90-week-old C57BL/6N male mice (n=10/group) were analyzed

FIG. 1E and FIG. 1F: IF staining of SLC25A51, insulin, glucagon, and DAPI; Scale bars, 50 μm.

FIG. 1G: Beta-Gal activity in pancreas tissue sections; Scale bars, 50 μm.

FIG. 1H: Body weight

FIG. 1I: random blood glucose levels,

FIG. 1J: serum SASP profile. One-way ANOVA; error bars are SEM. *p<0.05, **p<0.01, ****p<0.0001.

(FIG. 1K-1O): Pancreatic islet cells were isolated from 12- and 90-week-old C57BL/6N mice (n=5/group)

FIG. 1K: Glutamine levels

FIG. 1L: glutamate levels

FIG. 1M: Cellular ammonia levels

FIG. 1N and FIG. 1O: NAD⁺ and NADH levels and NAD⁺/NADH ratio. Two-tailed t-test; error bars are SEM. *p<0.05, **p<0.01, ***p<0.001.

FIG. 1P: 12-week-old and 90-week-old C57BL/6N male mice (n=8 per group) were sacrificed. Liver tissues were collected to analyze the expression of mitochondria-related genes, including Slc25a51 (left graph), Tom20, citrate synthase (CS), Slc25a38, and Slc25a53 (right graph). A two-tailed t-test was used to compare Slc25a51 expression between 12-week-old and 90-week-old mice. One-way ANOVA was used for comparisons of Tom20, CS, Slc25a38, and Sfxn1. Slc25a53 mRNA levels were not detected in liver tissues. Error bars represent the SEM. p<0.05.

FIG. 1Q: 12-week-old and 90-week-old C57BL/6N male mice (n=8 per group) were sacrificed. Pancreatic islets were collected to analyze the expression of mitochondria-related genes, including Slc25a51. A two-tailed t-test was used to compare Slc25a51 expression between 12-week-old and 90-week-old mice. Error bars represent the SEM. p<0.05.

FIG. 1R: 12-week-old and 90-week-old C57BL/6N male mice (n=8 per group) were sacrificed. Brain tissues were collected to analyze the expression of mitochondria-related genes, including Slc25a51 (left graph), Tom20, citrate synthase (CS), Slc25a38, and Slc25a53 (right graph). A two-tailed t-test was used to compare Slc25a51 mRNA expression levels between 12-week-old and 90-week-old mice. One-way ANOVA was used for comparisons of Tom20, CS, Slc25a38, Sfxn1, and Slc25a53 mRNA levels. Error bars represent the SEM. *p<0.05.

FIG. 2 shows that pancreatic islet cells isolated from S961-treated C57BL/6N mice are transcriptionally heterogeneous, and exhibit increased senescent beta-cell features marked by mitochondrial inner membrane transporter gene signatures.

Twelve-week-old C57BL/6N mice were bred and fed ad libitum until 28 weeks of age.

FIG. 2A: At 28 weeks, the mice were implanted with a mini-pump containing either vehicle or S961 (n=10).

FIG. 2B: Mice were monitored for 2 weeks to analyze random blood glucose levels

FIG. 2C: Mice were monitored for 2 weeks to analyze body weight; two-way repeated measures ANOVA; error bars represent SEM. ****p<0.0001.

FIG. 2D: Diabetes incidence were assessed

FIG. 2E: Plasma insulin levels were assessed

FIG. 2F: HOMA-IR were assessed

FIG. 2G: Serum SASP profiles (n=10/group) were assessed; one-way ANOVA; error bars are SEM. *p<0.05, ****p<0.0001.

FIG. 2H: After 2 weeks of monitoring, 12-week-old control (12W-C), 30-week-old control (30W-C), and 30-week-old S961-treated (30W-S) mice were sacrificed (n=10/group). Subsequently, beta-Gal, p16^{INK4A}, and insulin staining were performed in pancreatic islets on frozen sections and live cells using flow cytometry to confirm S961-induced b-cell senescence. Scale bars, 50 μm. One-way ANOVA; error bars are SEM. *p<0.05, **p<0.01, ****p<0.0001.

(FIG. 2I, FIG. 2 J) Using a second set of animals (n=10/group), pancreatic islet scRNA-seq was conducted. From this experiment, 21,853 qualified pancreatic islet cells were obtained from three groups.

FIG. 2I: The top five differentially expressed genes. An overview of the beta-cell senescence-induced diabetes islet cell atlas is presented, showing clusters (upper panel), group distributions (middle panel), and cluster annotation (lower panel). Cell-type annotation in scRNA-seq suggests clusters 0, 2, 5, 10, 11, 12, 14, 20, 21, and 23 represent pancreatic beta-cells.

FIG. 2J: The beta-cell senescence-induced diabetes islet cell atlas

FIG. 2K, FIG. 2L: Analyses in all clusters in three groups (12W-C, 30W-C, and 30W-S) were performed.

FIG. 2K: Cell number and proportion (%) were evaluated in each cluster and group.

FIG. 2L: GO (GO:CC, GO:BP, GO:MF) analyses in scRNA-seq data showed that 26 different clusters can be categorized into 12 different groups based on GO:CC.

FIG. 3 shows that SLC25A51 is part of a senescence gene signature in pancreatic beta cells.

Cluster annotation was performed using the CellKB^{PRO} program, as an unbiased approach, to filter out non-beta-cell and non-senescent clusters.

FIG. 3A: The filtered clusters (14, 20, 23, 10, and 21) were further evaluated for senescence characteristics using three different indices: beta-cell identity, beta-cell disallowed, and beta-cell senescence and aging-related genes. Additionally, a dendrogram was constructed to compare each cluster by analyzing the whole scRNA-seq data. Genes with positive Log 2FC and p-value (p<0.05) were counted for each index in a cluster to calculate the P.S. Using this method, early-stage senescence (cluster 20, 14), mid-stage (cluster 23, 10), and late-stage (cluster 21) were identified.

FIG. 3B: Slc25a51, Igf1r, and Nampt levels were analyzed in UMAP graphs.

FIG. 3C: Venn diagram analysis was performed between senescent beta-cell clusters (10, 14, 20, 21, and 23) to identify commonly shared genes.

FIG. 3D: Top box: Based on the GO:CC of clusters 10, 14, 20, 21, and 23 (mitochondrial inner membrane, transport), these clusters were re-annotated with mitochondrial inner membrane-related genes, including SLC25A family and ETC complex genes. Bottom diagram: A diagram depicting five different senescent beta-cell clusters, each annotated with its unique differentially expressed mitochondrial inner membrane transporter genes (DEGs). Each DEG is also illustrated in the diagram, showing how different mitochondrial inner membrane transporters facilitate the transport of specific metabolites in different senescent states.

FIG. 3E: Mouse pancreas tissues were stained for insulin, glucagon, and Slc25a51 in 12W-C, 30W-C, and 30W-S groups (n=10/group). Scale bars, 50 μm. One-way ANOVA; error bars represent SEM. ****p<0.0001.

FIG. 3F-FIG. 3J: Fresh pancreatic beta cells were isolated from 12W-C, 30W-C, and 30W-S (n=5/group)

FIG. 3F: Glutamine levels,

FIG. 3G: Glutamate levels

FIG. 3H: Cellular ammonia levels,

FIG. 3I: NAD+ and NADH levels

FIG. 3J: NAD+/NADH ratio. One-way ANOVA for comparison between 12W-C, 30W-C, and 30W-S. **p<0.01, ****p<0.0001, **p<0.01, ****p<0.0001.

FIG. 4 shows that SLC25A51 is upregulated in senescence downstream of the senescence-associated cell cycle regulator p16^{INK4A}, and modulation of intracellular NAD compartmentalization by SLC25A51 contributes to the senescence phenotype.

FIG. 4A-FIG. 4E: Thirty-week-old C57BL/6N male mice were sacrificed to isolate pancreatic beta cells and cultured with either PBS (control) or H₂O₂ (200 μM, 24 hours) (n=5/group).

FIG. 4A: beta-Gal activity,

FIG. 4B: SASP levels,

FIG. 4C mRNA levels of senescence markers (Igf1r, Cdkn2a, Trp53, Cdkn1a, Il1b, 116, Cxcl10) and Slc25a51 (n=5/group).

FIG. 4D: Protein levels of IGF1R, SLC25A51, MTCO2, and β-actin.

FIG. 4E: Mitochondrial membrane potential (TMRE), MitoTracker, and MitoSOX (n=5/group). Statistical analysis was performed using either (FIG. 4A) two-tailed t-test or (FIG. 4B, FIG. 4C, FIG. 4E) two-way ANOVA; error bars represent SEM; *p<0.05, **p<0.01, ***p<0.001, ****p<0.0001 for comparison between control (PBS) and H₂O₂-treated groups.

FIG. 4F: SLC25A51 expression levels and other protein levels were analyzed in MIN6 cells with replicative senescence by treating with H₂O₂ in a concentration-dependent manner with 0, 50, 100, 200, and 500 μM, or in a time-dependent manner (hour). At least three repeats of Western blot were performed for each experiment.

FIG. 4G, FIG. 4H: MIN6 cells overexpressed with pMXs-HA-Mito-GFP were generated to analyze mitochondrial NAD⁺ and NADH levels. Cells were treated with either control (PBS) or H₂O₂ (200 μM) in a time-dependent (0, 0.5, 1, 3, 24 hours) manner. Then, fresh whole-cell lysates and isolated mitochondria were obtained from each group to analyze

FIG. 4G: NAD⁺ and NADH levels at 0, 0.5, 1, 3, and 24 hours (n=5/group)

FIG. 4H: NAD⁺/NADH ratio at 0 and 24 hours, measured by a nanomolar-sensitive NAD⁺/NADH-Glo assay. Statistical analysis was performed using two-way ANOVA; error bars are SEM, *p<0.05, **p<0.01, ***p<0.001, ****p<0.0001.

FIG. 4I: SLC25A51 and other protein levels were analyzed in MIN6 cells passaged for 1, 4, 8, 12, and 16 weeks. At least three trials of Western blot were performed.

FIG. 4J: Six cells were irradiated with 10 Gy of ionizing radiation (IR) and analyzed at the indicated time points for SLC25A51 and other protein levels. At least three trials of Western blot were performed.

FIG. 4K: Six cells were treated with or without H₂O₂ (200 μM) in the presence or absence of Mito-Tempo (10 μM) to assess SLC25A51 and other protein levels. At least three trials of Western blot were performed.

FIG. 4L: Six cells were treated with either H₂O₂ (200 μM), Antimycin A (A. A., 0.5 μM, 24 hours), BSO (1 mM, 24 hours), Oligomycin A (Oligo., 0.5 μM, 24 hours), or Rotenone (Rot., 0.5 μM, 24 hours) to assess SLC25A51 and other protein levels. At least three trials of Western blot were performed.

FIG. 5 shows that SLC25A51 overexpression induces senescence markers in MIN6 cells

FIG. 5A: MIN6 cells were stably transfected with pLenti-Empty, pLenti-Cdkn1a (p21^{Cip1/Waf1}) pLenti-Cdkn2a (p16^{INK4A}), or pLenti-Nfe2l2 (Nrf2).

FIG. 5B: MIN6 cells were stably transfected with pLKO.1-shEmpty-tGFP or pLKO.1-shSLC25A51-tGFP treated with or without H₂O₂ (200 μM, 24 hours).

FIG. 5E: MIN6 cells were stably transfected with pMXs-Empty, pMXs-Flag-Slc25a51, or pMXs-Flag-Slc25a38.

(FIG. 5A, FIG. 5B, FIG. 5E): Whole-cell lysates from each condition were probed for SLC25A51, IGF1R, p16^{INK4A}, p21^{Cip1/Waf1}, p53, γ-H2AX, NRF2, MTCO2, TOMM20, citrate synthase, and D-actin.

Additionally, (FIG. 5A, FIG. 5E) Flag, SLC25A38, or (FIG. 5B) tGFP were tested. At least three trials of Western blot were performed.

(FIG. 5C, FIG. 5D, FIG. 5F, FIG. 5G): Fresh whole-cell lysates and mitochondria were isolated from each group

FIG. 5C and FIG. 5D: NAD⁺, NADH, and the NAD⁺/NADH ratio were analyzed in pLKO.1-shEmpty-tGFP or pLKO.1-shSLC25A51-tGFP cells. Statistical analysis was performed using two-way ANOVA; error bars are SEM, *p<0.05, **p<0.01.

FIG. 5F and FIG. 5G: NAD⁺, NADH, and the NAD⁺/NADH ratio were analyzed in HA-mito-GFP overexpressed pMXs-Empty, Flag-Slc25a51, or Flag-Slc25a51-K91A-mutant cells and

FIG. 6 shows that Beta cell-specific deletion of SLC25A51 improves age-associated glucose homeostasis and insulin resistance in mice

FIG. 6A: Human Slc25a51 levels were analyzed in T2D patients and healthy controls from publicly available datasets (E-M-5061, GSE87375).

FIG. 6B: The diagram depicts the strategy for infusing AAV6-INS1-CRE viruses through the pancreatic duct to specifically knock out beta-cell-specific SLC25A51 in 30-week-old SLC25A51 floxed C57BL/6N mice. After infusion and recovery, mice were analyzed for body weight, random blood glucose levels, b-hydroxybutyrate levels, IPGTT, and ITT under specified conditions and sacrificed after 15 weeks.

FIG. 6C-FIG. 6E: Pancreatic ductal infusion of either PBS, AAV6-INS1-Empty, AAV6-INS1-CRE was performed in each group. At 15 weeks after surgery, pancreatic islet cells and pancreatic tissues were isolated to confirm the knockout of SLC25A51 using Western blots and (FIG. 6J) IF staining.

FIG. 6C: Body weight analyzed twice a week.

FIG. 6D: Random blood glucose levels analyzed twice a week.

FIG. 6E: b-hydroxybutyrate levels were analyzed once a week for each mouse. Statistical analysis was performed using repeated measurement two-way ANOVA; error bars represent SEM; *p<0.05, **p<0.01 between PBS and AAV6-INS1-CRE groups, ^{#}p<0.05, ^{##}p<0.01 between AAV6-INS1-Empty and AAV6-INS1-CRE groups.

FIG. 6F-FIG. 6I: Six or eight weeks after surgery, mice were subjected to IPGTT and ITT. FIG. 6G: Graphs show blood glucose levels at each time point and area under the curve.

FIG. 6H: During IPGTT, serum was collected from each group at 0 and 15 min to measure serum insulin.

FIG. 6I After two weeks from IPGTT, ITT was performed to test insulin sensitivity. Two-way ANOVA; error bars represent SEM; **p<0.01 comparison between PBS and AAV6-INS1-CRE, ^{##}p<0.01 comparison between AAV6-INS1-Empty and AAV6-INS1-CRE. HOMA-IR was calculated for each group in male mice. HOMA-IR values between 0.5 and 1.4 are considered normal, ≥1.9 is regarded as early insulin resistance, and above 2.9 indicates insulin resistance.

FIG. 6J: After sacrifice, mouse pancreatic islets were acquired from each group. IF staining of (up) DAPI, insulin, Cox4, and Slc25a51, or (down) DAPI, insulin, and p16^{INK4A} were performed in pancreatic sections. Pancreatic sections were prepared as described; representative data are from 2-5 pancreatic islets per mouse in each group (n=35~40 islets/group). Scale bars, 20 μm. Two-way ANOVA; error bars are SEM. **p<0.01, ***p<0.001, ****p<0.0001.

FIG. 7

FIG. 7A: Human SLC25A51 mRNA levels were compared between different tissues from publicly available datasets (GTEx, Fantom5, and HPA).

FIG. 7B: Human cell lines before and after induction of senescence by extensive replication, oxidative stress, or DNA damage were analyzed for SLC25A51 mRNA levels (WI38: GSE130727; MSC, IMR90, HUVEC: GSE98440; HDF161: GSE93535; RPE-1: GSE83647; LS8817: GSE74620; MRC5: GSE64553). Human pancreatic islet cells (alpha, beta, PP, delta-cells) scRNA-seq data (GSE73727, GSE85241, GSE87375) were analyzed for SLC25A51 in RPKM levels.

FIG. 7C: Beta cells isolated from 12W, 30W, and 90W (n=5/group).

FIG. 7D: Beta cells isolated from 12W-C, 30W-C, and 30W-S groups (n=10/group) were stained with (FIG. 7C, FIG. 7D) insulin, (FIG. 7D) CD45, and beta-gal. Beta-gal frequency in pancreatic islets was analyzed in insulin+CD45-beta-gal+ cells from 10 mice per group. One-way ANOVA; error bars represent SEM. *p<0.05, ****p<0.0001.

FIG. 7E: Cell numbers of every cluster from scRNA-seq (FIG. 1) were compared between groups (12W-C, red; 30W-C, blue; 30W-S, green).

FIG. 7F and FIG. 7G: Senescence-associated secretory phenotype (SASP) was analyzed in mouse serum (n=10/group) using the Legendplex mouse proinflammatory chemokine panel.

FIG. 8.

FIG. 8A: Beta-cell identity genes, beta-cell disallowed genes, beta-cell senescence, and aging-related genes were analyzed in clusters 0, 5, 10, 14, 20, 21, and 23.

FIG. 8B: SLC25A51, Igf1r, Cdkn1a, Nfe2l2, and Nfe2l1 genes were compared in all 26 clusters. Expression of genes is in Log 2(Expression) (Log 2EXP).

FIG. 8C: Volcano plots for all 11,668 DEGs (up) and SLC25A family genes (bottom). The dotted line indicates FDR<0.05.

FIG. 8D: A total of 253 genes showed the GO term “mitochondrial inner membrane” in clusters 10, 14, 20, 21, and 23. With these 253 genes, STRING analysis indicated that these genes were involved in “mitochondrial inner membrane transport,” “mitochondria dynamics,” “mitochondrial ribosomal protein,” “oxidative phosphorylation,” and “fatty acid beta-oxidation.”

FIG. 8E: Diagram showing the unbiased approach to mine mitochondrial-related genes significant in senescence.

FIG. 9.

FIG. 9A: Slc25a51, Cdkn1a, Cdkn2a, and Igf1r expression levels (Log 2EXP) per cell were analyzed in each group (12W-C, 30W-C, 30W-S; n=21,853 cells). Average Log 2EXP expression values are in red for each gene and each group.

FIG. 9B and FIG. 9C: Slc25a51 expression levels in different groups (12W-C, 30W-C, 30W-S) and clusters (0-25 clusters) were compared in Log 2EXP.

FIG. 10.

To obtain an overview of systemic metabolic changes in (FIG. 10A-FIG. 10C) chronologically aged or (FIG. 10D-FIG. 10F) S961-treated mice, targeted metabolomics was performed on mouse serum (n=3/group). Serum samples were collected from 12-week (12W), 30-week (30W), and 90-week (90W) mice as well as from 12W-C, 30W-C, and 30W-S groups for metabolomics analysis.

FIG. 10A and FIG. 10D: Principal component analysis (PCA) revealed clear shifts in metabolite profiles among 12W, 30W, and 90W mice, as well as between 12W-C, 30W-C, and 30W-S groups driven by multiple components.

FIG. 10B and FIG. 10E: Using the MetaboAnalyst database, hierarchical clustering was performed between groups.

FIG. 10C: 30W and 90W groups were compared to analyze the overview of enriched metabolism pathways.

FIG. 10F: 30W-C and 30W-S groups were compared to analyze the overview of enriched metabolism pathways.

FIG. 10G: Mouse SLC25A51 enhancer region (SEQ ID NO: 1; RNA-seq intron features SEQ ID NOs: 7 and 8).

FIG. 10H: Predicted top 5 transcription factors for mouse SLC25A51 enhancer (top) and bona fide SLC25A51 target genes (bottom) analyzed by JASPAR (jaspar.genereg.net/) (MA0506.2.Nrf1: SEQ ID NO: 2; MA0162. 1.Egr1: SEQ ID NO: 3; MA01 7.1.Myc: SEQ ID NO: 4; MA0697.2.Zic3: SEQ ID NO:5; MA0609. 1.Crem: SEQ ID NO: 6).

FIG. 10I: Top 20 transcription factors analyzed using the PROMO program (alggen.lsi.upc.es/cgi-bin/promo_v3/promo/promoinit.cgi?dirDB=TF_8.3) for the human SLC25A51 gene.

FIG. 10J: To search for genes that may predict sensitivity or resistance to NRF2 activation, Weiss-Sadan et al. (Cell Metab 35, 487-503 e487. 10.1016/j.cmet.2023.01.012) performed a genome-wide CRISPR screen in the KEAP1-dependent cell line CALU6. Following infection with the sgRNA library, cells were grown for 11 population doublings in the presence of 1 μM of KI696 or vehicle control. As validation, NRF2 appeared as the top-scoring gene that mediated resistance when depleted in KEAP1-1-dependent CALU6 cells. Among many oxidative phosphorylation-related genes, SLC25A51, SLC25A22, NDUFB1, and SLC25A1 were enriched in KEAP1-dependent CALU6 cells.

FIG. 10K: Slc25a51, Nfe2l1 (Nrf1), and Nfe2l2 (Nrf2) expression levels were analyzed by UMAP in pancreatic islet scRNA-seq from FIG. 2.

FIG. 11.

FIG. 11A: Pancreatic islet cells were isolated from 30-week-old C57BL/6N male mice (n=5/group). The cells were treated with H₂O₂ and stained for beta-Gal activity; Two-way ANOVA, error bars represent SEM, *p<0.05, ***p<0.001, ****p<0.0001.

FIG. 11B: MIN6 cells were treated with H₂O₂ in a time- and concentration-dependent manner to probe for mitochondrial ETC complexes.

FIG. 11C: MIN6 cells were transfected with lentiviral vectors to generate pMXs-Empty and pMXs-HA-GFP;

FIG. 11D: pLKO.1-shEMPTY and pLKO.1-shSLC25A51 MIN6 cells. Each stable cell line was assessed for GFP or tGFP signals using flow cytometry.

FIG. 11E: Mitochondria were isolated from different stable cell lines and then probed for Tom20 and beta-actin.

(FIG. 11F-FIG. 11H) pMXs-HA-Mito-GFP-transfected MIN6 cells were treated with PBS, FK866 (2 nM, 24 hours), or H₂O₂ (200 μM, 24 hours). Cells were processed to isolate whole-cell lysates and purified mitochondria by MITO-IP.

FIG. 11F: Samples were probed for mitochondrial matrix protein (citrate synthase), mitochondrial OMM protein (Tom20), mitochondrial IMM proteins (SLC25A51, SLC25A38), cytosolic protein (beta-actin), and ER protein (calreticulin).

FIG. 11G: Metabolites extracted from whole-cell lysates to perform LC-MS analysis (n=3/group)

FIG. 11H: Metabolites extracted from purified mitochondria to perform LC-MS analysis (n=3/group). Two-way ANOVA, error bars represent SEM, *p<0.05, **p<0.01, ***p<0.001, ****p<0.0001.

FIG. 12

FIG. 12A: A diagram depicting the generation of SLC25A51-floxed C57BL/6N mice using a CRISPR-Cas9-based approach by Biocytogen Pharmaceuticals.

FIG. 12B and FIG. 12C: SLC25A51 expression per cell was compared between T2D patients and healthy controls using two different human beta-cell scRNA-seq datasets (E-MTAB-5061, GSE81608). Heatmaps of Cdkn1a (p21) and Cdkn2a (p16) expression were also analyzed in both datasets.

FIG. 12D: 30-week-old SLC25A51-floxed C57BL/6N male mice were infused with eitherAAV6-INS1-Empty or AAV6-INS1-CRE viruses via pancreatic ductal infusion. Mice were sacrificed 15 weeks post-surgery. SLC25A51, p16^{INK4A}, MTCO2, and beta-actin protein expression levels were analyzed in pancreatic beta cells isolated from 45-week-old INS1-EMPTY (n=2) and INS1-CRE (n=4) groups.

FIG. 12E: Pancreatic ductal infusion of either PBS, AAV6-INS1-Empty, or AAV6-INS1-CRE was performed in each group of female C57BL/6N mice (n=5-6 per group).

FIG. 12F: Random blood glucose levels measured twice a week.

FIG. 12G: body weight measured twice a week.

FIG. 12H: beta-hydroxybutyrate levels measured once a week for each mouse.

FIG. 12I-FIG. 12K: Six or eight weeks post-surgery, female mice underwent an IPGTT and ITT.

Graphs show blood glucose levels at each time point and area under the curve (FIG. 12I, FIG. 12J).

FIG. 12I: During the IPGTT, serum was collected from each group at 0 and 15 min.

FIG. 12J: Two weeks after the IPGTT, an ITT was performed to test insulin sensitivity. Two-way ANOVA; error bars represent SEM; **p<0.01.+++

FIG. 12K: Serum insulin levels measured.

FIG. 13: 30-week-old C57BL/6N wild-type (WT) and β-cell-specific Slc25a51 knockout male mice (INS2-KO) (n=5 per group) were sacrificed. Pancreatic islets were isolated and treated with or without Nutlin-3a (10 μg/mL, 6 h) or CDDO-Im (10 nM, 6 h) to activate p53 and Nrf2, respectively. Samples were then collected for qPCR analysis of Slc25a51 and CRE mRNA expression levels. One-way ANOVA was used for comparisons of Slc25a51 and CRE mRNA expression levels. Error bars represent the SEM. *p<0.05, **p<0.01, ***p<0.001.

FIG. 14

FIG. 14A: 12-week-old and 1-year-old C57BL/6N wild-type (WT) and β-cell-specific Slc25a51 knockout male and female mice (Slc25a51^{βKO}) were generated and maintained on a 9% fat diet throughout life.

FIG. 14B: Body weight, fat mass, and lean mass (n=5 per group), as well as random blood glucose levels (n=10 per group), were measured in the mice generated as shown in FIG. 14A. Two-way ANOVA was used for comparisons. Error bars represent the SEM. *p<0.05, ** p<0.01, ***p<0.001, ****p<0.0001.

(FIGS. 14C-14H): 1-year-old wild-type (WT, white) and β-cell-specific Slc25a51 knockout male mice (Slc25a51^{βKO}, red) were maintained on a 9% fat diet throughout life.

FIG. 14C: Pancreatic islets were isolated and stained with MitoTracker, MitoSox, and TMRE. A two-tailed t-test was used for comparisons. Error bars represent the SEM. *p<0.05, ***p<0.001.

FIG. 14D: 1-year-old wild-type and Slc25a51^{βKO} male mice (n=5 per group) were sacrificed, and pancreatic islets were isolated, with islets from each mouse placed in separate wells. Glucose-stimulated insulin secretion (GSIS) assays were performed by sequential incubation in low (2.8 mM) and high (17.5 mM) glucose in Krebs Ringer Bicarbonate buffer. Supernatants were collected at each step, and insulin levels were quantified by ELISA using insulin standards.

FIG. 14E: Pancreatic islets were isolated and were evaluated for oxygen consumption rate (OCR) with a Seahorse extracellular flux analyzer XFe96 (Agilent). Freshly isolated pancreatic islets were first placed in each well with low glucose (2 mM) for 30 minutes in non-C02 incubator. After recording basal cellular respiration, cells were treated sequentially with glucose (20 mM), oligomycin (2 ug/ml), and rotenone/antimycin A (2 uM). Basal and glucose-stimulated OCR was compared between groups.

FIG. 14F: Whole pancreatic islets (WI) were isolated from each mouse, and mitochondria were pooled from five mice per sample to achieve a mitochondrial protein concentration of approximately 0.5 mg/mL. Whole-islet NAD⁺ and NADH levels were quantified using NAD⁺/NADH Glo kit (Promega) with NAD⁺/NADH standards and normalized to protein content determined by BSA standards. Mitochondrial NAD⁺ and NADH levels were measured in a similar manner, with normalization accounting for pooling from five mice per sample. After obtaining NAD⁺ and NADH levels, NAD⁺/NADH ratio was calculated. A two-tailed t-test was used for comparisons. Error bars represent the SEM. *p<0.05, **p<0.01, ****p<0.0001.

FIG. 14G: Whole pancreatic islets (WI) were isolated from each mouse, and then, glutamine and glutamate levels were quantified using Glutamine/Glutamate Glo kit (Promega) with standards. A two-tailed t-test was used for comparisons. Error bars represent the SEM. *p<0.05.

FIG. 14H: Mice were sacrificed, and pancreatic tissues were collected and fixed in 4% paraformaldehyde (PFA). Fixed tissues were embedded in paraffin and processed for H&E staining. A two-tailed t-test was used for comparisons. Error bars represent the SEM. *p<0.05, **p<0.01, ****p<0.0001.

FIG. 14I: Mice were sacrificed, and pancreatic tissues were collected and snap-frozen in OCT compound with 2-methylbutane. Snap-frozen tissues were subjected to beta-galactosidase staining. A two-tailed t-test was used for comparisons. Error bars represent the SEM. *p<0.05, **p<0.01, ****p<0.0001.

### DETAILED DESCRIPTION OF THE INVENTION

The present disclosure provides methods for detecting the presence of cellular senescence in a subject. The methods include detecting the level of a biomarker comprising solute carrier family 25 member 51 (SLC25A51) in a biological sample from the subject and comparing the level of SLC25A51 with a control sample, wherein an increase in the level of SLC25A51 in the subject as compared to the control sample indicates the presence of cellular senescence in the subject. The methods also include detecting the level of a biomarker comprising nicotinamide adenine dinucleotide (NAD+) in a biological sample from the subject and comparing the level of NAD+ with a control sample, wherein a decrease in the level of NAD+ in the in the subject as compared to the control sample indicates the presence of cellular senescence in the subject.

Additionally, the present disclosure provides methods for improving pancreatic β-cell function in a subject, for treating a metabolic disorder (e.g., a metabolic disorder associated with cellular senescence, e.g. diabetes) in a subject, for increasing the survival of pancreatic β-cells during β-cell transplantation therapy in a subject, and for delaying the onset of senescence in pancreatic β-cells of a subject. The methods include administering to the subject a therapeutically effective amount of an agent that decreases the level of SLC25A51 (e.g., an SLC25A51 inhibitor) in the subject. In some embodiments, the subject has a metabolic disorder (e.g., a metabolic disorder associated with cellular senescence, e.g., diabetes).

### I. Methods of the Invention

### A. Methods for Detecting the Presence of Cellular Senescence

In one aspect, the present disclosure provides a method for detecting the presence of cellular senescence in a subject, the method comprising: (a) detecting the level of one or more biomarkers comprising solute carrier family 25 member 51 (SLC25A51) or nicotinamide adenine dinucleotide (NAD+) in a biological sample from the subject; and (b) comparing the level of the one or more biomarkers with a control sample, wherein an increase in the level of SLC25A51 in the subject as compared to the control sample indicates the presence of cellular senescence in the subject; and/or wherein a decrease in the level of NAD+ in the in the subject as compared to the control sample indicates the presence of cellular senescence in the subject.

As used herein, the term “senescence” or “cellular senescence” refers to a state of a cell in which the cell remains viable and metabolically active but has lost the ability to proliferate (see, for example, Herranz et al. The Journal of clinical investigation 128.4 (2018): 1238-1246; Hernandez-Segura et al, Trends in cell biology 28.6 (2018): 436-453). Senescence is characterized by stable cell cycle arrest in which proliferating cells become resistant to growth-promoting stimuli. Senescence can be induced by a wide range of intrinsic and extrinsic insults, including DNA damage, oncogenic activation, oxidative stress, inflammation, mitochondrial dysfunction, chemotherapeutic agents, and exposure to UV irradiation and ionizing radiation. In response to these stimuli, senescent cells undergo multiple phenotypic changes such as morphology changes, chromatin remodeling, and metabolic reprogramming, and secrete a complex mix of factors termed the senescence-associated secretory phenotype (SASP). Hyperglycemia is an important driver of senescence, cell identity loss, and beta-cell dysfunction, though the mechanism remains unclear. Increased glycolytic rates have been shown to drive adaptation in NAD⁺ compartmentalization, affecting pancreatic beta cell identity and function.

Senescent cells influence multiple biological processes in disease and aging, as well as normal tissue homeostasis. Senescence protects against tumorigenesis by stably arresting mitotic cells, such as fibroblasts or immune cells, but it also contributes to changes in metabolic activities and gene expression associated with inflammation and aging. Postmitotic cells, which are unreplaceable or infrequently replaced with age, including neurons, pancreatic beta cells and oocytes, are particularly vulnerable to metabolic stress and can also become senescent.

Cells that have undergone senescence may be characterized by one or more features such as formation of γ-H2AX (a phosphorylated form of the histone variant H2AX) nuclear foci; an increase in the expression and/or level of p16^{INK4A}, an increase in the expression and/or level of p16^{INK4B}; an increase in the expression and/or level of p21^{Cip1/Waf1}; increased activity of senescence associated β-galactosidase (SA-β-Gal); production of DNA senescence-associated heterochromatic foci (SAHF); loss of proliferation; trimethylation of histone 3 lysine 9 (H3K9me3); endoplasmic reticulum stress and induction of the unfolded protein response; increased glucose consumption; cell enlargement; persistent DNA damage response (DDR) signaling; and formation of DNA segments with chromatin alterations reinforcing senescence (DNA-SCARS). Cellular senescence is also associated with increased expression and/or secretion of pro-inflammatory cytokines called the senescence associated secretory phenotype (SASP), which include, but are not limited to, CXCL10, CCL4, CCL3, CXCL1, CCL5, eNAMPT, CCL2, CCL4, CXCL2, CXCL3, CCL20, CCL11, CCL17, CCL22, CXCL9, CXCL13, CXCL5, IL-6, and IL-1(3. Cells that have undergone cellular senescence typically have increased levels of p16^{INK4A} expression compared to the level of P16^{INK4A} expression in cells that have not undergone cellular senescence. Cells that have undergone cellular senescence typically have increased levels of SA-β-Gal activity compared to cells that have not undergone cellular senescence.

In some embodiments, markers of senescence in pancreatic β-cells include, but are not limited to, changes in the expression levels of Ulk3, Arntl, Igf1r, GIs, Mapk14, Cdkn1a, Trp53, Map2k1, Sirt1, Id2, Il6, Cdkn2a, Il1a, Prkcd, Bambi, Plaur; Gls2, Cav1, Tbx2, Tbx3 or Il1b, compared to a pancreatic β-cell that has not undergone senescence. In some embodiments, markers of senescence in pancreatic β-cells are changes in the expression level of one or more genes selected from the group consisting of Igf1r Cdkn2a, Trp53, and Il-1β.

As used herein, “solute carrier family 25 member 51” or “SLC25A51” refers to the well-known mitochondrial membrane carrier protein that mediates the import of NAD+ into mitochondria. SLC25A51 plays a critical role in regulating mitochondrial NAD+ levels and intracellular NAD+ compartmentalization. Through its role in shifting cellular NAD+ compartmentalization, SLC25A51 has also been shown to alter NAD+-dependent metabolic pathways, DNA repair, and cancer cell proliferation by uncoupling the mitochondrial NAD+/NADH ratio.

Exemplary nucleotide and amino acid sequences of SLC25A51 can be found, for example, at GenBank Accession No. NM_033412.4 (Homo Sapiens SLC25A51); GenBank Accession No. NM_001009949.3 (Mus musculus SLC25A51); GenBank Accession No. NM_001024785 (Rattus Norvegicus SLC25A51); and GenBank Accession No. NM_001194642 (Macaca mulatta SLC25A51).

Additional examples of SLC25A51 mRNA sequences are readily available through publicly available databases, e.g., GenBank, UniProt and OMIM. Further information on SLC25A51 can be found, for example, at www.ncbi.nlm.nih.gov/gene/?term=SLC25A51.

The entire contents of each of the foregoing GenBank Accession numbers and the Gene database numbers are incorporated herein by reference as of the date of filing this application.

SLC25A51 is expressed in various tissues and cell types, and has diverse functions in metabolic regulation through its role in mitochondrial NAD⁺ transport and intracellular compartmentalization. Increased SLC25A51 levels correlate with elevated tumor growth or cancer cell proliferation. In AML patients, increased SLC25A51 levels are associated with poor survival outcomes, and lowering SLC25A51 levels prevented tumor growth in U937-engrafted female mice. Similar observations have been made in other types of cancer. In human thoracic aortic aneurysm, SLC25A51 expression levels inversely correlate with disease severity and postoperative progression.

As a mitochondrial NAD⁺ transporter, SLC25A51 plays a key role in regulating intracellular NAD⁺ distribution and redox ratio. Its activity influences not only mitochondrial NAD⁺ levels, but also nucleo-cytoplasmic NAD⁺ distribution in certain cell types. This regulation affects processes such as the epigenetic state, DNA repair, and metabolic function through sirtuins, PARPs, and AMPK, as well as cell cycle regulation and senescence through p53, p16^{INK4}, and p2^{Cip1/Waf1}. Modulating mitochondrial NAD⁺ transporters such as Ndt1/Ndt2 has been shown to extend chronological lifespan in yeast and Arabidopsis plants by enhancing mitochondrial respiration efficiency and reducing ROS production. Notably, recent studies on beta-cell aging found that NAD⁺-related genes (Nrk1, Nmnat2) are upregulated with aging and disrupt the intracellular NAD⁺/NADH ratio. In rapidly proliferating cancer cells, SLC25A51 is upregulated to decouple mitochondrial NAD⁺/NADH, providing a proliferative advantage by supporting oxidative reactions from a variety of fuels.

As used herein, the term “biomarker” refers to a measurable substance or characteristic that indicates a specific biological process, disease state, or response to treatment. Biomarkers may include proteins, peptides, nucleic acids (e.g., RNA), or chemicals. In some embodiments, a biomarker is a protein (e.g., SLC25A51 protein). In some embodiments, a biomarker is a peptide (e.g., a peptide derived from the SLC25A51 protein). In some embodiments, a biomarker of the present disclosure is an mRNA (e.g., SLC25A51 mRNA). In some embodiments, a biomarker is a chemical (e.g., NAD+).

The level of SLC25A51 protein may be determined using any method known in the art for the measurement of protein levels. Such methods include, for example, Western blotting, enzyme-linked immunosorbent assays (ELISAs), immunofluorescent assays, electrochemiluminescence assays, high performance liquid chromatography (HPLC), absorption spectroscopy, colorimetric assays, spectrophotometric assays, flow cytometry, immunoelectrophoresis, radioimmunoassay (RIA), and the like.

The level of SLC25A51 mRNA that is expressed by a cell or group of cells (e.g., pancreatic β-cells may be determined using any method known in the art for assessing mRNA expression. In some embodiments, the level of expression of SLC25A51 in a sample is determined by detecting a transcribed polynucleotide, or portion thereof, e.g., mRNA of the SLC25A51 gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene™ (PreAnalytix™, Switzerland). In some embodiments, the level of expression of SLC25A51 is determined using a nucleic acid probe that is capable of selectively binding to a specific SLC25A51.

In some embodiments, determining the level of SLC25A51 mRNA in a sample involves the process of nucleic acid amplification or reverse transcriptase (to prepare cDNA) of for example mRNA in the sample, e.g., by RT-PCR, followed by the detection of the amplified molecules using techniques well known to those of skill in the art. These detection schemes are especially useful for the detection of nucleic acid molecules if such molecules are present in very low numbers. In some embodiments, the level of expression of SLC25A51 is determined by quantitative RT-PCR.

The level of NAD+ in a sample may be determined using any method known in the art for the quantification of NAD+ amounts. Such methods include, for example, enzymatic assays measured by methods such as fluorescence, colorimetry or luminescence. In some embodiments, the levels of NAD+ are determined by methods involving mass spectrometry (MS) (e.g., tandem mass spectrometry (MS/MS)). In some embodiments, the MS is coupled to liquid chromatography (e.g., ultra-high-pressure LC).

In some embodiments, methods of the disclosure further comprises determining the NAD+/NADH ratio in the sample. The NAD+/NADH ratio in a sample may be determined using any method known in the art for the quantification of NAD+, NADH and NAD+/NADH amounts. Such methods include, but are not limited to, colorimetric, fluorometric or luminescent assays which assess cellular metabolic activity, redox state, and energy production. Exemplary assays include the NAD/NADH-Glo™ Assay (Promega).

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs.

In some embodiments, samples may be derived from blood. In some embodiments, samples may be derived from testis, breast, pancreas, or liver tissue of the subject. In certain embodiments, samples are derived from the pancreatic tissue of the subject. In some embodiments, samples derived from pancreatic tissue comprise pancreatic islet cells. In some embodiments, the pancreatic islet cells comprise pancreatic β-cells. In some embodiments, the sample comprises cell lysates of a cell or a group of cells (e.g., cells derived from a subject).

A control sample that may be used to assess the level of one or more biomarkers (e.g., SLC25A51, e,g, NAD+) includes a sample from a cell or a group of cells that have not undergone senescence. In some embodiments, the control sample may be an earlier-in-time sample derived from the subject (e.g., a human or animal subject). In some embodiments, the control sample may be obtained from a younger subject.

In some embodiments, the level of one or more biomarkers is compared to a predetermined threshold value. In some embodiments, the predetermined threshold value is determined from an earlier-in-time sample derived from the subject. In some embodiments, the predetermined threshold value is determined from a younger subject. In some embodiments, the predetermined threshold value is determined from a cell or a group of cells that have not undergone senescence.

In some embodiments, an increase in the level of SLC25A51 in the subject as compared to a control sample indicates the presence of cellular senescence in the subject. In some embodiments, an increase of at least 1.1 fold, at least 1.5 fold, at least 2 fold, at least 5 fold, at least 10 fold, at least 20 fold, at least 50 fold, at least 100 fold, at least 200 fold in the level of SLC25A51 in the subject as compared to a control sample indicates the presence of cellular senescence in the subject.

In some embodiments, a decrease in the level of NAD+ in the subject as compared to a control sample indicates the presence of cellular senescence in the subject. In some embodiments, a decrease of at least 1.1 fold, at least 1.5 fold, at least 2 fold, at least 5 fold, at least 10 fold, at least 20 fold, at least 50 fold, at least 100 fold, at least 200 fold in the level of NAD+ in the subject as compared to a control sample indicates the presence of cellular senescence in the subject.

In some embodiments, a decrease in the NAD+/NADH ratio in the subject as compared to a control sample indicates the presence of cellular senescence in the subject. In some embodiments, a decrease of at least 1.1 fold, at least 1.5 fold, at least 2 fold, at least 5 fold, at least 10 fold, at least 20 fold, at least 50 fold, at least 100 fold, at least 200 fold in the NAD+/NADH ratio in the subject as compared to a control sample indicates the presence of cellular senescence in the subject.

The methods of the disclosure further comprise determining the levels of one or more molecules selected from the group consisting of P16^{INK4A} P21^{Cip1/Waf1}, γ-H2AX, NAMPT, IGF1R, glutamine, glutamate, cellular ammonia and serum secretory associated phenotype (SASP) factors. Exemplary methods for the measurement of glutamate and glutamine levels include the Glutamine/Glutamate-Glo™ Assay (Promega).

In some embodiments, an increase in the levels of one or more molecules selected from the group consisting of P16^{INK4A}, P21^{Cip1/Waf1}, γ-H2AX, IGF1R, NAMPT, glutamine, glutamate, cellular ammonia and serum secretory associated phenotype (SASP) factors as compared to a control sample indicates the presence of cellular senescence in the subject. In some embodiments, an increase of at least 1.1 fold, at least 1.5 fold, at least 2 fold, at least 5 fold, at least 10 fold, at least 20 fold, at least 50 fold, at least 100 fold, at least 200 fold in the levels of one or more molecules selected from the group consisting of P16^{INK4A}, P21^{Cip1/Waf1}, γ-H2AX, IGF1R, NAMPT, glutamine, glutamate, cellular ammonia and serum secretory associated phenotype (SASP) factors in the subject as compared to a control sample indicates the presence of cellular senescence in the subject.

SASP factors senescence associated secretory phenotype (SASP) are factors (e.g., inflammatory cytokines, immune modulators, growth factors and proteases) secreted by senescent cells. In some embodiment, SASP factors comprise one or more of CXCL10, CCL4, CCL3, CXCL1, CCL5, eNAMPT, CCL2, CCL4, CXCL2, CXCL3, CCL20, CCL11, CCL17, CCL22, CXCL9, CXCL13, CXCL5, IL-6, and IL-1β. In some embodiments, an increase of at least 1.1 fold, at least 1.5 fold, at least 2 fold, at least 5 fold, at least 10 fold, at least 20 fold, at least 50 fold, at least 100 fold, at least 200 fold in one or more SASP factors in the subject as compared to a control sample indicates the presence of cellular senescence in the subject.

As used herein, the term “upregulated” in the context of gene expression refers to an increase in the expression of a gene relative to its endogenous levels of expression. In some embodiments, the expression of the gene is increased by at least 1.1 fold, at least 1.5 fold, at least 2 fold, at least 5 fold, at least 10 fold, at least 20 fold, at least 50 fold, at least 100 fold, at least 200 fold compared to its endogenous expression level. In some embodiments, gene expression levels are quantified by measuring corresponding mRNA levels (e.g., by PCR-based techniques, RNA-seq, Northern blotting, fluorescence in situ hybridization (FISH), etc.)

### B. Methods for Improving Pancreatic β-Cell Function

In another aspect, the present disclosure provides a method of improving pancreatic β-cell function in a subject in need thereof, the method comprising administering to the subject a therapeutically effective amount of an SLC25A51 inhibitor to the subject.

Pancreatic beta cells, or pancreatic β-cells are specialized endocrine cells located within the pancreatic islets of Langerhans, and are responsible for the production and release of insulin and amylin. Pancreatic β-cells play a vital role in maintaining blood glucose levels.

As used herein, the term “improving pancreatic β-cell function” includes one or more of the following: increasing insulin sensitivity, decreasing insulin resistance, maintaining glucose homeostasis, increasing glucose clearance, lowering circulating insulin levels, lowering glucose levels (e.g., blood glucose levels) in a subject.

In some embodiments, administration of the SLC25A51 inhibitor improves glucose clearance in the subject. Glucose clearance is a measure of how quickly glucose is removed from the bloodstream and is typically calculated by dividing glucose utilization by plasma glucose levels. In some embodiments, glucose clearance is measured by a glucose tolerance test. In some embodiments, the blood glucose level in the subject after administration of the SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control.

In some embodiments, administration of the SLC25A51 inhibitor increases insulin sensitivity in the subject. Insulin-sensitivity describes the level of insulin that is necessary to keep a constant blood glucose level maintained by insulin-susceptible cells. In some embodiments, insulin sensitivity is measured by an insulin tolerance test, for example, a test during which insulin is injected into a subject's vein, after which blood glucose is measured at regular intervals. In some embodiments, the circulating insulin level in the subject after administration of SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control.

In some embodiments, the body weight of the subject after administration of the SLC25A51 inhibitor is at least 1.1 fold, at least 1.5 fold, at least 2 fold lower compared to the control.

In some embodiments, administration of the SLC25A51 inhibitor delays the onset of senescence in pancreatic β-cells of the subject. In some embodiments, the SLC25A51 inhibitor delays the onset of senescence in pancreatic β-cells as a result of aging. In some embodiments, the SLC25A51 inhibitor delays the onset of senescence in pancreatic β-cells following exposure to one or more external or internal stimuli such as DNA damage, oncogenic activation, oxidative stress, inflammation, mitochondrial dysfunction, chemotherapeutic agents, and exposure to UV irradiation and ionizing radiation.

In some embodiments, pancreatic β-cells that have been administered an SLC25A51 inhibitor progress to senescence at a later time point than control cells (e.g, pancreatic β-cells that have not been administered an SLC25A51 inhibitor). In some embodiments, the onset of senescence in pancreatic f-cells that have been administered an SLC25A51 inhibitor is delayed by at least 1.1 fold, at least 1.5 fold, at least 2 fold, at least 5 fold, at least 10 fold, at least 20 fold, at least 50 fold, at least 100 fold, at least 200 fold compared to the control.

In some embodiments, the expression levels of one or more genes selected from the group consisting of CDKN2A, CDKN1A, CXCL10, IL-1β, and IL-6 in a subject after administration of the SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control.

In some embodiments, the levels of p16^{INK4A}-positive senescent beta cells in a subject after administration of the SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control.

In some embodiments, the control is measured in the subject (e.g., in a sample derived from the subject) prior to administration of the SLC25A51 inhibitor.

In another aspect, the present disclosure provides a method for delaying the onset of senescence in a cell or a group of cells (e.g., pancreatic β-cells), the method comprising administering to the cell an SLC25A51 inhibitor, thereby delaying the onset of senescence in the cell or group of cells.

In another aspect, the present disclosure provides a method for inducing the onset of senescence in a cell or a group of cells (e.g., pancreatic β-cells), the method comprising increasing the expression of SLC25A51 in the cell or group of cells relative to endogenous SLC25A51 expression, thereby inducing the onset of senescence in the cell or group of cells.

As used herein, the term “inhibit” refers to a decrease in expression, stability, and/or a biological activity of SLC25A51. For example, the term “inhibit” refers to the ability to decrease or down-regulate the expression, stability, and/or activity of SLC25A51.

An “inhibitor of SLC25A51” or an “SLC25A51 inhibitor” as used herein, includes any molecule that partially or fully blocks, inhibits, or neutralizes a biological activity mediated by SLC25A51. In some embodiments, an SLC25A51 inhibitor comprises a small molecule, an antibody, an antisense oligonucleotide, an siRNA, an shRNA, a miRNA or combinations thereof. In some embodiments, an SLC25A51 inhibitor as described below, decreases the expression, stability, and/or activity of SLC25A51 by at least 1.1 fold, at least 1.5 fold, at least 2 fold, at least 5 fold, at least 10 fold, at least 20 fold, at least 50 fold, at least 100 fold, at least 200 fold, compared to measurements made in the absence of the inhibitor.

In some embodiments, the methods of the present invention comprise administering a therapeutically effective amount of an SLC25A51 inhibitor to the subject. The therapeutically effective amount of an SLC25A51 inhibitor is an amount sufficient to treat disease, e.g., a disease associated with cellular senescence, e.g., diabetes. The therapeutically effective dosage of an SLC25A51 inhibitor will vary somewhat from subject to subject, and will depend upon factors such as the age, weight, and condition of the subject and the route of delivery. Such dosages can be determined in accordance with procedures known to those skilled in the art. In an exemplary embodiment, a therapeutically effective amount of an SLC25A51 inhibitor is an amount effective to reduce the level or activity of SLC25A51 in a subject.

The term “subject” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject is a human, such as a human being treated or assessed for a disease, disorder or condition, a human at risk for a disease, disorder or condition, a human having a disease, disorder or condition, and/or human being treated for a disease, disorder or condition as described herein. In some embodiments, the subject has a metabolic disorder. In some embodiments, the subject has a metabolic disorder associated with cellular senescence. In some embodiments, the subject has diabetes. In some embodiments, the subject has Type 1 or Type 2 diabetes. In some embodiments, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age.

### C. Methods of Treating a Metabolic Disorder

In another aspect, the present disclosure provides a method of treating a metabolic disorder associated with cellular senescence in a subject, the method comprising administering to the subject a therapeutically effective amount of an SLC25A51 inhibitor to the subject.

As used herein, a “metabolic disorder” or “metabolic disease” refers to a disease or a disorder that alters normal metabolic processes. A metabolic disorder may occur due to altered chemical reactions, altered processing and distribution of macronutrients. Cellular senescence is an important risk factor for multiple metabolic diseases through mechanisms such as promoting systemic inflammation, oxidative stress, and metabolic dysregulation. Metabolic disorders associated with cellular senescence include diabetes, metabolic syndrome (MetS), metabolic cardiovascular diseases, osteoporosis, atherosclerosis, hepatic steatosis, osteoarthritis, idiopathic pulmonary fibrosis, obesity-induced anxiety, tau-mediated neurodegenerative disease, chronic kidney disease (see, for example, Zhang et al, Front. Cell Dev. Biol (2023); Spinelli et al, J Clin Invest. 2023; 133(12):e169922). In some embodiments, a metabolic disorder associated with cellular senescence is diabetes.

Diabetes mellitus or diabetes, is a group of endocrine diseases characterized by high blood glucose levels or hyperglycemia. Common symptoms of diabetes include increased thirst, frequent urination, extreme hunger, and unintended weight loss.

The distinct types of diabetes include Type 1 diabetes (T1D), Type 2 diabetes (T2D) and gestational diabetes. T1D arises from an autoimmune disorder that leads to the destruction of insulin-producing pancreatic β-cells. In T1D, the β-cells are destroyed, resulting in deficiency of insulin. Histologically, T1D is characterized by an immune infiltrate within or around the pancreatic islets (insulitis), consisting of large numbers of mononuclear cells and CD8+ T cells. In contrast to TiD, T2D is non-autoimmune-mediated and is characterized by insulin resistance in peripheral tissues, such as skeletal muscle and adipose tissues and/or pancreatic β-cell dysfunction. With disease progression, pancreatic β-cell dysfunction is gradually exacerbated, leading to pancreatic β-cell destruction, increased severity of diabetic symptoms, and the need for insulin replacements therapies in this patient population.

In some embodiments, administration of the SLC25A51 inhibitor to a subject with a metabolic disorder (e.g., a metabolic disorder associated with cellular senescence, e.g. diabetes) improves glucose clearance in the subject. In some embodiments, glucose clearance is measured by a glucose tolerance test. In some embodiments, the blood glucose level in the subject after administration of the SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control.

In some embodiments, administration of the SLC25A51 inhibitor a subject with a metabolic disorder (e.g., a metabolic disorder associated with cellular senescence, e.g. diabetes) increases insulin sensitivity in the subject. In some embodiments, the circulating insulin level in the subject after administration of SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control.

The term “treatment” or “treating” as used herein, refers to an intervention that imparts a benefit to a subject, e.g., a subject afflicted with or at risk for developing a disease. Treating includes actions taken and actions refrained from being taken for the purpose of improving the condition of the patient, e.g., the relief of one or more symptoms, or delay in the onset or progression of the disease.

In another aspect, the present disclosure provides a method of increasing the survival of pancreatic β-cells during β-cell transplantation therapy in a subject, the method comprising administering to the subject a therapeutically effective amount of an SLC25A51 inhibitor.

β-cell transplantation, or islet cell transplantation, is a therapy for diabetes (e.g., type 1 diabetes) which transplants insulin-producing cells to a subject to restore endogenous insulin production. In some embodiments, administration of an SLC25A51 inhibitor increases the survival of pancreatic β-cells during β-cell transplantation therapy by at least 1.1 fold, at least 1.5 fold, at least 2 fold, at least 5 fold, at least 10 fold, at least 20 fold, at least 50 fold, at least 100 fold, at least 200 fold lower compared to control.

In some embodiments, the control is measured in a subject who has not been administered the SLC25A51 inhibitor.

### II. SLC25A51 Inhibitors

Molecules which inhibit the expression and/or activity of SLC25A51 are useful in the methods of the present invention. An inhibitor of SLC25A51 can be a nucleic acid, a polypeptide, an antibody, or a small molecule compound. In some embodiments, the inhibitor or activator functions at a level of transcription, mRNA stability, translation, protein stability/degradation, protein modification, and protein binding.

### A. Inhibitory Nucleic Acids

In some embodiments, the methods of the disclosure include inhibiting SLC25A51 (e.g., inhibiting SLC25A51 activity or expression) using inhibitory nucleic acids. A nucleic acid inhibitor can encode a small interference RNA (e.g., an RNAi agent) that targets SLC25A51 and inhibits its expression or activity. The term “RNAi agent” refers to an RNA, or analog thereof, having sufficient sequence complementarity to a target RNA to direct RNA interference. Examples also include a DNA that can be used to make the RNA. Exemplary nucleic acid inhibitors of SLC25A51 include, but are not limited to, MISSION® esiRNA targeting human SLC25A51 (Millipore Sigma), MISSION® shRNA Lentiviral particles (Millipore Sigma), MCART1 siRNA (h) and shRNA Plasmid (h), MCART1 CRISPR Plasmids (h).

RNA Interference: RNA interference (RNAi) refers to a sequence-specific or selective process by which a target molecule (e.g., a target gene, protein or RNA) is down-regulated. Generally, an interfering RNA (“RNAi”) is a double stranded short interfering RNA (siRNA), short hairpin RNA (shRNA), or single-stranded micro-RNA (miRNA) that results in catalytic degradation of specific mRNAs, and also can be used to lower or inhibit gene expression. RNA interference (RNAi) is a process where double-stranded RNA (dsRNA) induces the sequence-specific regulation of gene expression in animal and plant cells and in bacteria (Aravin and Tuschl, FEBS Lett. 26:5830-5840, 20 2005; Herbert et al., Curr Opin. Biotech. 19:500-505, 2008; Sharp, Genes Dev, 15:485-490, 2001; Valencia-Sanchez et al. Genes Dev 20:515-524, 2006). In mammalian cells, RNAi can be triggered by 21-nucleotide (nt) duplexes of small interfering RNA (siRNA) (Chiu et al., Mol. Cell. 10:549-561, 2002), by microRNA (miRNA), functional small-hairpin RNA (shRNA), or other dsRNAs which are expressed in vivo using DNA templates with RNA polymerase II or III promoters (Zeng et al., Mol. Cell 9:1327-1333, 2002; Paddison et al., Genes Dev 16:948-958, 2002; Denti, et al., Mol. Ther 10:191-199, 2004; Lee et al., Nature Biotechnol. 20:500-505, 2002; Paul et al., Nature Biotechnol. 20:505-508, 2002; Rossi, Human Gene Ther 19:313-317, 2008; Tuschl, T., Nature Biotechnol. 20:440-448, 2002; Yu et al., Proc. Natl. Acad. Sci. USA 99(9):6047-6052, 30 2002).

siRNA Molecules: The term “short interfering RNA” or “siRNA” or “small interfering RNA” refers to an RNA agent, preferably a double-stranded agent, of about 10-50 nucleotides in length, preferably between about 15-25 nucleotides in length the strands optionally having overhanging ends comprising, for example 1, 2 or 3 5 overhanging nucleotides (or nucleotide analogs), which is capable of directing or mediating RNA interference.

In general, the methods described herein can use dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can be transcribed in vitro or in vivo, e.g., shRNA, from a DNA template. The dsRNA molecules can be designed using any method known in the art.

The methods described herein can use both siRNA and modified siRNA derivatives, e.g., siRNAs modified to alter a property such as the specificity and/or pharmacokinetics of the composition, for example, to increase half-life in the body, e.g., crosslinked siRNAs. Thus, the invention includes methods of administering siRNA derivatives that include siRNA having two complementary strands of nucleic acid, such that the two strands are crosslinked. The oligonucleotide modifications include, but are not limited to, 2-O-methyl, 2′-fluoro, 2′-O-methyoxyethyl and phosphorothioate, boranophosphate, 4′-thioribose. (Wilson and Keefe, Curr Opin. Chem. Biol. 10:607-614, 2006; Prakash et al., J Med. Chem. 48:4247-4253, 2005; Soutschek et al., Nature 30 432:173-178, 2004).

Modifying siRNA derivatives at a 3′ terminus (e.g., with a peptide, nanoparticle, biotin molecule) may improve cellular uptake or enhance cellular targeting activities of the resulting siRNA derivative, be useful for tracing the siRNA derivative in the cell, or improve the stability of the siRNA derivative compared to the corresponding siRNA. The inhibitory nucleic acid compositions can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.: 47(1), 99-112, 2001; Fattal et al., J. Control Release 53(1-3):137-43, 1998; Schwab et al., Ann. Oncol. 5 Suppl. 4:55-8, 1994; and Godard et al., Eur J. Biochem. 232(2):404-10, 1995).

siRNA Delivery: Direct delivery of siRNA in saline or other excipients can silence target genes in tissues, such as the eye, lung, and central nervous system. Liposomes and nanoparticles can also be used to deliver siRNA into animals. Delivery methods using liposomes, e.g. stable nucleic acid-lipid particles (SNALPs), dioleoyl phosphatidylcholine (DOPC)-based delivery system, as well as lipoplexes, e.g. Lipofectamine 2000, TransIT-TKO, have been shown to effectively repress target mRNA (de Fougerolles, Human Gene Ther 19:125-132 (2008); Landen et al., Cancer Res. 65:6910-6918 (2005); Luo et al., Mol. Pain 1:29 (2005); Zimmermann et al., Nature 441:111-114 (2006)). Conjugating siRNA to peptides, RNA aptamers, antibodies, or polymers, e.g. dynamic polyconjugates, cyclodextrin-based nanoparticles, atelocollagen, and chitosan, can improve siRNA stability and/or uptake. Viral-mediated delivery mechanisms can also be used to induce specific silencing of targeted genes through expression of siRNA, for example, by generating recombinant adenoviruses harboring siRNA under RNA Pol II promoter transcription control. Engineered RNA precursors, introduced into cells or whole organisms will lead to the production of a desired siRNA molecule.

Antisense: An “antisense” nucleic acid can include a nucleotide sequence that is complementary to a “sense” nucleic acid encoding a protein, e.g., complementary to the coding strand of a double-stranded cDNA molecule or complementary to a target mRNA sequence. The antisense nucleic acid can be complementary to an entire coding strand of a target sequence, or to only a portion thereof (for example, the coding region of a target gene). In another embodiment, the antisense nucleic acid molecule is antisense to a “noncoding region” of the coding strand of a nucleotide sequence encoding the selected target gene (e.g., the 5′ and 3′ untranslated regions).

An antisense nucleic acid can be designed such that it is complementary to the entire coding region of a target mRNA but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences relating to SLC25A51 (e.g, GenBank Accession No. NM_033412.4 (Homo Sapiens SLC25A51); GenBank Accession No. NM_001009949.3 (Mus musculus SLC25A51); GenBank Accession No. NM_001024785 (Rattus Norvegicus SLC25A51); and GenBank Accession No. NM_001194642 (Macaca mulatta SLC25A51)), one of skill in the art can easily choose and synthesize any of a number of appropriate antisense molecules for use in accordance with the present invention.

The antisense nucleic acid molecules of the invention are typically administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

CRISPR Technology: The expression of a target polynucleotide (e.g., DNA or RNA of SLC25A51) can be modified by allowing a CRISPR complex to bind to the polynucleotide, wherein the CRISPR complex comprises a CRISPR enzyme complexed with a guide sequence hybridized to a target sequence within the target polynucleotide, wherein the guide sequence is linked to a tracr mate sequence which in turn hybridizes to a tracr sequence. In some embodiment, binding of CRISPR complex to a target polynucleotide results in an increased expression of the target polynucleotide. In another embodiment, binding of CRISPR complex to a target polynucleotide results in a decreased expression of the target polynucleotide (e.g., DNA or RNA of SLC25A51).

The clustered, regularly interspaced, short palindromic repeat (CRISPR) technology is included in the invention as an approach for generating RNA-guided nuclease with customizable specificities for targeted genome editing. Genome editing mediated by these nucleases has been used to rapidly, easily and efficiently modify endogenous genes in a wide variety of biomedically important cell types and in organisms that have traditionally been challenging to manipulate genetically.

In general, the term “CRISPR system” refers collectively to transcripts and other/elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In embodiments of the invention the terms guide sequence and guide RNA are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides (e.g., DNA or RNA of SLC25A51).

In some embodiments, the CRISPR/Cas system is a type II CRISPR system and the Cas enzyme is Cas9, which catalyzes DNA cleavage. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence NGG following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archae, Mol. Cell 2010, 37(1): 7.

### B. Antagonist Antibodies

The invention further contemplates methods and compositions comprising an antagonist antibody which inhibits SLC25A51, thereby decreasing expression and/or activity of SLC25A51 and improving pancreatic β-cell function, and/or treating diabetes in a subject. In some embodiments, the antagonist antibody of SLC25A51, or antigen binding portion thereof, decreases the expression and/or activity of SLC25A51.

Antagonist antibodies suitable for use in the methods of the present invention may be identified, screened for (e.g., using phage display), or characterized for their physical/chemical properties and/or biological activities by various assays known in the art (see, for example, Antibodies: A Laboratory Manual, Second edition, Greenfield, ed., 2014). Binding specificity of an antibody for its antigen can be tested by known methods in the art such as ELISA, Western blot, or surface plasmon resonance.

Antibodies may be produced using recombinant methods and compositions known in the art, e.g., as described in U.S. Pat. No. 4,816,567, incorporated by reference herein. An isolated nucleic acid encoding, for example, an anti-SLC25A51 antibody is used to transform host cells for expression. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): a vector comprising a nucleic acid that encodes an amino acid sequence comprising the VL of the antibody and an amino acid sequence comprising the VH of the antibody, or a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell).

For recombinant production of an anti-SLC25A51 antibody, a nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR.sup.-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### C. Small Molecule Inhibitors

Other inhibitory modulators that can be used to specifically inhibit the activity of an SLC25A51 protein are chemical compounds that directly inhibit SLC25A51 activity. Such compounds can be either natural products or members of a combinatorial chemistry library, and can be identified using screening assays known in the art. Exemplary small molecule inhibitors of SLC25A51 include fludarabine phosphate (Li et al, Cell Death Differ (2023) Jul. 7; 30(8):1916-1930).

### III. Pharmaceutical Formulations

Pharmaceutical formulations comprising SLC25A51 inhibitors of the present invention may be prepared by mixing the protein or nucleic acid having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween™, Pluronics™ or polyethylene glycol (PEG).

The active ingredients may also be packaged in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes. Generally, the ingredients of compositions are supplied either separately or mixed together in unit dosage form, for example, as a dry lyophilized powder or water free concentrate in a hermetically sealed container such as an ampoule or sachet indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration. In an alternative embodiment, one or more of the pharmaceutical compositions of the invention is supplied in liquid form in a hermetically sealed container indicating the quantity and concentration of the agent.

The active agent can be incorporated into a pharmaceutical composition suitable for parenteral administration, typically prepared as an injectable solution. The injectable solution can be composed of either a liquid or lyophilized dosage form in a flint or amber vial, ampule or pre-filled syringe. The liquid or lyophilized dosage may further comprise a buffer (e.g., L-histidine, sodium succinate, sodium citrate, sodium phosphate or potassium phosphate, sodium chloride), a cryoprotectant (e.g., sucrose trehalose or lactose, a bulking agent (e.g., mannitol), a stabilizer (e.g., L-Methionine, glycine, arginine), an adjuvant (hyaluronidase).

The compositions of this invention, e.g., a SLC25A51 inhibitor, may be in a variety of forms. These include, for example, liquid, semi-solid and solid dosage forms, such as liquid solutions (e.g., injectable and infusible solutions), microemulsion, dispersions, liposomes or suspensions, tablets, pills, powders, liposomes and suppositories. The preferred form depends on the intended mode of administration and therapeutic application. Pharmaceutical compositions comprising SLC25A51 inhibitors described herein may be formulated for administration to a particular tissue. For example, in certain embodiments, it may be desirable to administer SLC25A51 to pancreatic tissue.

SLC25A51 inhibitors suitable for use in the methods of the present invention can be administered by any suitable means, including parenteral administration (e.g., injection, infusion), and may be administered by subcutaneous, intraperitoneal, intrapulmonary, and intranasal, and, if desired for local treatment, intralesional administration, e.g., intratumoral administration. Parenteral infusions include intravenous, intraarterial, intraperitoneal, intramuscular, intradermal or subcutaneous administration. In addition, SLC25A51 inhibitors can be suitably administered by pulse infusion, particularly with declining doses. The dosing can be given by injections, such as intravenous or subcutaneous injections. The route of administration can be selected according to various factors, such as whether the administration is brief or chronic. Other administration methods are contemplated, including topical, particularly transdermal, transmucosal, rectal, oral or local administration e.g. through a catheter placed close to the desired site. Injection, especially intravenous, is of interest.

In some embodiments, the methods of the present invention comprise administering a therapeutically effective amount of an SLC25A51 inhibitor to the subject. The therapeutically effective amount of SLC25A51 is an amount sufficient to treat disease (e.g., a metabolic disorder, e.g., diabetes), in a subject. The therapeutically effective dosage of a SLC25A51 inhibitor will vary somewhat from subject to subject, and will depend upon factors such as the age, weight, and condition of the subject and the route of delivery. Such dosages can be determined in accordance with procedures known to those skilled in the art. In an exemplary embodiment, a therapeutically effective amount of an SLC25A51 inhibitor is an amount effective to reduce the level or activity of a SLC25A51 in a subject.

In some embodiments, SLC25A51 inhibitors are administered in combination with an additional therapeutic. In some embodiments, the additional therapeutic is administered to treat diabetes. In some embodiments, the additional therapeutic comprises one or more of insulin (e.g., short-acting insulin, long-acting insulin, intermediate-acting insulin, rapid-acting insulin, combination insulin), amylinomimetic injectables (e.g., Pramlintide), metformin, dipeptidyl peptidase 4 (DPP-4) inhibitors (e.g., Alogliptin, Linagliptin, Saxagliptin, Sitagliptin), glucagon-like peptide 1 (GLP-1), dual glucose-dependent insulinotropic polypeptide (GIP), GLP-1 receptor agonists (e.g., Dulaglutide, Exenatide, exenatide extended-release, Liraglutid, Lixisenatid, injectable semaglutide), sodium-glucose cotransporter 2 (SGLT2) inhibitors (e.g., Bexagliflozin, canagliflozin, dapagliflozin, and empagliflozin), sulfonylureas (e.g., glimepiride, glipizide, glyburide), thiazolidinediones (TZDs) (e.g., Rosiglitazone and pioglitazone), alpha glucosidase inhibitors (e.g., Acarbose, miglitol), bile acid sequestrants (e.g., colesevelam), dopamine-2 agonists (e.g., Bromocriptine) and meglitinides (e.g., Nateglinide, repaglinide)

In some embodiments, the therapeutic methods described herein are performed on a human subject.

### EXAMPLES

### Example 1: Methods

Animal Housing: Animal experiments were conducted in the specific pathogen-free environment at the animal facility at Seoul National University Hospital with approval from the Institutional Animal Care and Use Committee (IACUC no. 21-0089-S1A0) and at the Harvard Center for Comparative Medicine, Harvard Medical School, Harvard University with approval from the IACUC (no. IS00003204). Mice were maintained on a 12-hour light/dark cycle and were fed PicoLab Mouse Diet 20 (5058), formulated with 20% protein and 9% fat.

C57BL/6N Mice: To avoid confounding effects from the loss of nicotinamide nucleotide transhydrogenase (Nnt), which could contribute to differences in metabolic phenotype, Nnt-wild-type C57BL/6N mice were employed instead of C57BL/6J mice, which have a known mutation in Nnt exon 7-11. Twelve-, 30-, and 90-week-old C57BL6/N male mice were acquired from Joongah Bio (Korea) or from wild-type littermates produced from SLC25A51 heterozygote floxed mice. Random blood glucose levels (ad libitum) and body weight were measured twice a week for SLC25A51-floxed mice infused with either PBS, AAV6-INS1-Empty, or AAV6-INS1-CRE. For S961-treatment experiments, random blood glucose levels and body weight were measured once a day for 14 days. Circulating β-hydroxybutyrate levels were measured once a week for SLC25A51-floxed mice infused with PBS, AAV6-INS1-Empty, or AAV6-INS1-CRE. After sacrifice, pancreatic tissues were collected from mice, stored in optimal cutting temperature (OCT) compound, or fixed in 4% paraformaldehyde for further analyses. Isolation of pancreatic islet cells, splenocytes, and hepatocytes from C57BL/6N mice was performed as described below.

Generation of SLC25A51 fl/fl C57BL/6N Mic: Slc25a51 floxed C57BL/6N mice were generated using a CRISPR/Cas9-based approach by Biocytogen Pharmaceuticals. Briefly, two single-guide RNAs (sgRNAs) were designed using the CRISPR design tool www.sanger.ac.uk/htgt/wge/) to target regions upstream of the exon 3 and downstream of the 3′UTR. These were screened for on-target activity using a Universal CRISPR Activity Assay (UCATM, Biocytogen Pharmaceuticals). To minimize random integrations, a circular donor vector was used. The gene targeting vector, containing a 5′ homologous arm, a target fragment (part of the 5′UTR, exon 3 coding region, and 3′UTR), and a 3′ homologous arm, served as a template to repair the double-strand breaks generated by Cas9/sgRNA. Two LoxP sites were precisely inserted on both sides of the target fragment of the Slc25a51 gene. T7 promoter sequence was added to the Cas9 or sgRNA template via PCR amplification in vitro. Cas9 mRNA, the targeting vector, and sgRNAs were co-injected into the cytoplasm of one-cell-stage fertilized C57BL/6N eggs. The injected zygotes were transferred into the oviducts of Kunming pseudo-pregnant females to generate F0 mice. F0 mice with the expected genotype, confirmed by tail genomic DNA PCR and sequencing, were mated with C57BL/6N mice to establish germline-transmitted F1 heterozygous mice. F1 heterozygous mice were genotyped using tail genomic PCR, Southern blot, and DNA sequencing.

AAV6 Virus Production, Infusion, and Surgical Procedures: The rat INS1 promoter sequence was inserted into an AAV6-CRE backbone, which is commonly used for pancreatic ductal infusion. Subsequently, AAV6-INS1-Empty (1.81×10¹³ GC/ml) and AAV6-INS1-CRE (7.16×10¹³ GC/ml) were produced by the Penn Vector Core, University of Pennsylvania School of Medicine. For the pancreatic ductal infusion of AAV6 in SLC25A51 fl/fl mice, 1012 GC/ml per mouse was infused (30-week-old mouse body weight: 45~50 g, infusion rate: 6 μl/min, volume: 180 μl).

scRNA-seq of Mouse Pancreatic Islet Cells: Chromium Next GEM Single Cell 3′ v3.1 was used for scRNA-seq on mouse pancreatic islet cells. Libraries were prepared using the Chromium controller according to the 10× Chromium Next GEM Single Cell 3′ v3.1 protocol (CG000204). Briefly, the cell suspensions were diluted in nuclease-free water to achieve a targeted cell count of 5,000. The cell suspension was mixed with a master mix and loaded with Single Cell 3′ v3.1 Gel Beads and Partitioning Oil into a Chromium Next GEM chip G. RNA transcripts from single cells were uniquely barcoded and reverse-transcribed within droplets. cDNA molecules were pooled and underwent end repair, the addition of a single ‘A’ base, and adapter ligation. The products were purified and enriched by PCR to create the final cDNA library. The purified libraries were quantified using qPCR according to the qPCR Quantification Protocol Guide (KAPA) and qualified using the Agilent Technologies 4200 TapeStation (Agilent Technologies). Libraries were then sequenced using the HiSeq platform (Illumina). Read lengths used were 28 for Read1 (Cell barcode & UMI length), 10 for i7 and i5 index (sample index), and 90 for Read2 (insert).

Evaluating the Progression of Senescence for Beta-Cell Clusters: The filtered clusters (14, 20, 23, 10, and 21) were further evaluated for senescence characteristics using three different indices: beta-cell identity, beta-cell disallowed genes, and beta-cell senescence and aging-related genes. Additionally, a dendrogram analysis was performed to compare each cluster by analyzing the complete scRNA-seq data. Genes with a positive Log 2FC and p-value (p<0.05) were counted for each index in a cluster to calculate the Progression of Senescence (P. S.). The total number of beta-cell identity genes with a positive Log 2FC and p-value (p<0.05) was subtracted by the sum of beta-cell disallowed and beta-cell senescence genes with a positive Log 2FC and p-value (p<0.05). Using this method, early-stage senescence (clusters 20 and 14), mid-stage (clusters 23 and 10), and late-stage (cluster 21) were identified.

Global Metabolomics Profiling Using LC-QTOF-MS: Freshly collected mouse plasma samples were diluted (85:15, sample:mixture; v/v) with an acetonitrile:methanol:water (3:3:4) mixture (HPLC-grade, Millipore, Bedford, MA, USA) and 10% methanol, respectively. Each sample (5 μL) was loaded onto an Acquity UPLC BEH C18 (1.7 μm, 2.1 mm×100 mm; Waters Corp., Milford, MA, USA) column and analyzed using an Agilent 6530 QTOF mass spectrometer (Agilent Technologies). The experimental procedure was performed by the Seoul National University Hospital Metabolomics Core Facility. The average % coefficient of variance of analytes ranged from 15%-30%. Quantified measurements were analyzed using the MetaboAnalyst program. All samples were examined simultaneously in a single run to avoid batch effects, and the overall quality of the analysis procedure was monitored using repeat extracts of a pooled plasma sample (Biocrates, AbsoluteIDQ p180 kit).

Mitochondria Isolation: Mitochondria were immunopurified from MITO-HA-GFP-expressing MIN6 cells (30 million cells per sample) following the Mito-IP protocol. Mitochondria from shEmpty-tGFP and shSLC25A51-tGFP MIN6 cells were isolated using the Mitochondria Isolation Kit for Cultured Cells (Thermo Fisher Scientific).

### Quantification of NAD⁺ and NADH

LC-MS/MS in Mitochondria Isolated From HA-Tagged MIN6 Cells: For small polar metabolite (e.g., NAD⁺, NADH) separation and data acquisition on mitochondria samples (diluted in a 40:40:20 methanol:acetonitrile:water mixture with 0.5% formic acid), a Vanquish Horizon (Thermo Fisher Scientific, Waltham, MA) ultra-high-pressure liquid chromatography (LC) system coupled to a Thermo Exploris 480 mass spectrometer (MS) was used. For separation, a Waters (Milford, MA) XBridge BEH Amide (2.5 μm, 2.1×150 mm) column fitted with a VanGuard (2.5 μm, 2.1×5 mm) column guard (Waters, Milford, MA) was used. The mobile phases were as follows: Phase A: 95% water/5% acetonitrile and Phase B: 20% water/80% acetonitrile with 10 mM ammonium acetate and 10 mM ammonium hydroxide in both phases. The flow rate was held constant at 0.3 mL/min, and the following gradient conditions were used: 0 min, 100% B; 3 min, 100% B; 3.2 min, 90% B; 6.2 min, 90% B; 6.5 min, 80% B; 10.5 min, 80% B; 10.7 min, 70% B; 13.5 min, 70% B; 13.7 min, 45% B; 16 min, 45% B; 16.5 min, 100% B; and 22 min, 100% B. The samples were held at 4 °C, the injection volume was 5 μL, the column was held at 25 °C, and the run time was 22 min. The separated metabolites were analyzed on the MS in both positive and negative ionization modes in the same run (switching mode) with the Full MS scan detection mode. The mass spectra were acquired using the following parameters: resolution=120,000, m/z range=70-1,000, AGC Target: Standard, Maximum Injection Time: Standard, and run time=22 min. The ElectroSpray Ionization source parameters for both modes were as follows: Ion Transfer Tube 325 °C, Vaporizer 300 °C, Spray Voltage 3.5 kV (+)/2.5 kV (−), Sheath Gas 50 (arbitrary units), Aux Gas 10 (arbitrary units), Sweep Gas 1 (arbitrary units), and RF Lens 50%.

Glutamine Glutamate-Glo, Ammonia, NAD⁺/NADH-Glo measurements in Pancreatic Islet Cells and Cultured MIN6 Cells: For Glutamine/Glutamate-Glo assay (Promega) and Ammonia assay kit (Abcam), cell lysates from mouse pancreatic islet cells were analyzed according to the manufacturer's instructions. For NAD measurements in MIN6 cells, thirty million HA-Mito-GFP-expressing MIN6 cells (pMXs-EMPTY-HA-Mito-GFP, FLAG-SLC25A51-HA-Mito-GFP, FLAG-SLC25A51-K91A-HA-Mito-GFP) or shNTC-tGFP and shSLC25A51-tGFP cells were collected for each group. Mitochondrial isolation was performed as described above, and then the NAD⁺/NADH-Glo assay (Promega) was conducted according to the manufacturer's instructions with μl of the mitochondrial fraction.

S961 Treatment In vivo Prior to the insertion of an osmotic pump, mice were evenly matched and randomized into groups considering their baseline body weights and blood glucose concentrations. Vehicle (PBS) or 20 nmol S961 diluted in PBS was loaded into an Alzet osmotic pump 2001 and implanted subcutaneously on the back of either 10- or 28-week-old mice, with pumps changed weekly for a total of two weeks.

IPGTT ITT and Insulin Measurements Prior to the IPGTT, C57BL/6N mice were transferred to clean cages with no food for overnight fasting (12 hr). Mice were weighed to calculate the volume of 20% glucose solution required for intraperitoneal injection (10 μL×body weight in g), and random blood glucose levels were measured at 0 min. Before and after 20% glucose injection, blood was collected from the submandibular vein. The collected serum was stored at −80 °C to analyze insulin content within 2 weeks (Ultra Mouse Insulin Elisa Kit, Crystal Chem). For the ITT, C57BL/6N mice were transferred to clean cages and fasted for 4-6 hours before the test. To prepare the insulin solution, two stock solutions were required. First stock: Dilute 50 μL Humulin R 100 U/mL (3.5 mg/mL) to 4,950 μL of cold, sterile 1×PBS. Gently invert the solution 3-4 times to mix. Second stock: Dilute 1 mL of the first stock in 9 mL of cold, sterile 1×PBS. Again, gently invert solution 3-4 times to mix. Each mouse was weighed to determine the appropriate dose of the insulin solution, which was administered via intraperitoneal injection. Blood glucose levels were measured and recorded at 15, 30, 60, 90, and 120 min after the initial glucose delivery.

SA-Beta-Gal Staining. The SA-beta-Gal staining procedure was followed. Briefly, OCT-embedded, snap-frozen, unfixed tissues were cryosectioned at a thickness of 10 μm and stored at −80 °C for 24 hours. All tissues were processed and stained within 2-3 days after sacrifice. For SA-beta-Gal staining of frozen tissue sections, pancreatic sections were thawed, fixed, and stained using the Senescence Beta-Galactosidase Staining Kit (Cell Signaling, #9860). For live cell SA-beta-Gal staining using FACS, the CellEvent™ Senescence Green Flow Cytometry Assay Kit (Thermo, #C10840) was used following the manufacturer's protocol.

Primary Cell Isolation (Islet Beta Cell, Splenocyte, and Hepatocyte Isolation): Mice were euthanized, and freshly prepared collagenase P (Roche, Indianapolis, IN) solution (0.5 mg/mL) was injected into the pancreas via the common bile duct. The perfused pancreas was digested at 37 °C for 15 min, and the islets were handpicked under a stereoscopic microscope. Cells were then dispersed into a single-cell suspension. Furthermore, pancreatic islet cells were gated, sorted, and purified according to forward and side scatter, with the percentage of beta cells by insulin staining ranging from 85%-90%, as previously reported. For splenocyte and hepatocyte isolation, the process was conducted as previously reported.

IF Analyses on Pancreatic Tissue Samples: C57BL/6N mice were sacrificed, and pancreases were obtained and preserved in OCT compound for frozen cryotome sectioning. Pancreases embedded in a frozen block were prepared in 5 μm cross-sections for immunofluorescence (IF) staining. Sections were blocked with 5% goat serum (Jackson ImmunoResearch, 005-000-121) in antibody diluent (Dako) for 30 min and incubated overnight at 4 °C with the following antibodies: Insulin, Glucagon, or SLC25A51 (Thermo, BS24079R), Cox4 (Proteintech, 66110-1-1g), and p16^{INK4A} (Abcam, Ab54210, Ab211542). After washing with TBST, slides were incubated for 1 hour with the appropriate fluorochrome-attached secondary antibody (Jackson ImmunoResearch). Slides were rinsed and analyzed after mounting with a cover glass. Images were acquired using a confocal microscope (TCS-SP8, Leica, magnification 20×). For quantification, islet images were captured systematically, covering the whole section in confocal mode to evaluate every cluster of insulin-stained cells (3-7 cells) or islets (8 or more cells)/section for SLC25A51 intensity (AU); sections were coded and read blindly. For better microscopic resolution and color contrast, image channels were digitally merged, magnified, and assigned artificial colors using commercially available software programs.

Flow Cytometry, Intracellular Staining, or Sorting in Tissue-Derived Primary Cells: Isolated mouse beta cells, preadipocytes, splenocytes, or hepatocytes were dispersed using EDTA-PBS for 15 min at 37 °C and were resuspended in FACS buffer (0.5% BSA (Millipore) in PBS). Using a FACS Canto (BD), pancreatic islet cells were gated according to forward scatter, with the percentage of beta cells by insulin staining ranging from 85%-90%. For detecting preadipocytes, splenocytes, and hepatocytes, the gating strategy described in previous reports was followed. For the evaluation of beta-Gal activity, a fluorescent substrate from the flow cytometry assay kit (Thermo, C10841) was used following the manufacturer's protocol. For intracellular cytokine staining, cells were stained with or without surface markers (e.g., CD45) and then fixed and permeabilized using the Intracellular Fixation & Permeabilization Buffer Set (eBioscience). After permeabilization, an antibody cocktail (IL-1β, CXCL10, IL-6, Insulin) with different fluorochromes was added for intracellular staining. Cells were washed and analyzed in FACS buffer. Data were analyzed using FlowJo software (Treestar, Ashland).

SASP Analysis in Mouse Serum Using the Multiplex-Cytokine Assay: Mouse serum obtained from chronologically aged mice (12W, 30W, 90W) and treatment groups (12W-C, 30W-C, 30W-S) was stained and analyzed using a multiplexed bead-based assay for mouse proinflammatory chemokines (Legendplex, 740446, BioLegend).

Cell Lines and Plasmid: MIN6 cells were cultured in DMEM supplemented with 20% fetal bovine serum (FBS). HEK293FT cells for virus production were grown in DMEM supplemented with 10% FBS. Cells were incubated in a humidity-controlled environment at 37 °C with 5% CO2 (Thermo Fisher Scientific Forma 371) and were used up to 10 passages. pGenLenti-empty, pGenLenti-CDKN1A, pGenLenti-CDKN2A, and pGenLenti-NFE2L2 were obtained from Genscript. pMXs-FLAG-SLC25A38 and pMXs-SOD2-CAT-GFP vectors were generated in this study. pLKO.1-tGFP-shNTC (non-targeting control, SHC016) and pLKO.1-tGFP-shSLC25A51 were purchased from Mission shRNA Custom DNA (Millipore Sigma).

Virus Production in HEK293FT Cells: HEK293FT cells were co-transfected with the VSVG envelope plasmid, retroviral packaging plasmids Gag-Pol, and pMXs, pLKO.1, or pLenti plasmids. The plasmids included pMXs-HA-MITO-GFP (Addgene, #83356), pMXs-FLAG-SLC25A51 (Addgene, #133247), pMXs-FLAG-SLC25A51-K91A (Addgene, #133248), pMXs-FLAG-SLC25A38 (Addgene, #218412), pMXs empty vectors, pLKO.1-Neo-CMV-tGFP-shNTC (non-targeting control; Mission shRNA, Millipore, SH016), pLKO.1-Neo-CMV-tGFP-shSLC25A51 (Mission shRNA, Millipore), pLenti-Nfe2l2, pLenti-Cdkn2a, or pLenti-Cdkn1a (Genscript). Transfections were performed using 2.0×10⁶ cells in 6-well plates in RPMI containing polybrene (10 g/mL; Millipore). The culture medium was changed 8 hours after transfection. The virus-containing supernatant was collected 48 hours after transfection, spun, and filtered to eliminate debris and cells. Subsequently, virus-containing supernatants were used to generate MIN6 stable cell lines.

Generation of MIN6 Stable Cell Line: MIN6 cells were transfected with different viruses collected from HEK293FT cultured media, as described above. After an 18-hour incubation with the virus, the medium was changed to fresh culture medium containing the appropriate selection reagent (pMXs: blasticidin, pLenti: puromycin, pLKO.1: neomycin) and selected for 72 hours. MIN6 cells with HA-GFP or tGFP were sorted using a FACS Aria sorter (BD).

MIN6 Cell Sorting: For sorting MIN6 stable cell lines, GFP-positive (pMXs-HA-GFP or pLKO.1-NEO-CMV-tGFP-shEMPTY or pLKO.1-NEO-CMV-tGFP-shSLC25A51) cells were acquired and sorted using a FACS Aria cell sorter (BD). For evaluating mitochondrial morphology and function, TMRE, MitoTracker, and MitoSOX were used to stain either MIN6 or pancreatic beta cells. Cells were washed and analyzed in FACS buffer. Data were analyzed using FlowJo software (Treestar, Ashland). mRNA Quantification by qPCR Total RNA was purified using the RNeasy Mini Kit (Qiagen) as per the manufacturer's protocol. cDNA was synthesized by reverse transcription of 1 g of total RNA using AMV Reverse Transcriptase (Promega) following the manufacturer's instructions. Quantitative real-time PCR was performed in a 20 μL reaction mixture containing 1 μL of cDNA, 10 pM of each primer, and 10 L of SYBR Select Master Mix (Thermo Fisher Scientific) using an Applied Biosystems 7500 Real-Time PCR System (Thermo Fisher Scientific).

Western Blot Analysis: Cell lysates were subjected to electrophoresis using 4-20% precast gels (Thermo Fisher Scientific). The resolved gels were transferred to PVDF membranes, blocked with 5% skimmed milk in Tris-buffered saline containing 0.05% Tween-20 (TBS-T), and incubated with primary antibodies against SLC25A51 (Mouse, Thermo BS-24079R), SLC25A38 (Abcam, ab133614), IGF1R (Cell Signaling, 9750S), p16^{INK4A} (Novus, NBP2-37736), p21^{cip1/waf1} (Invitrogen 14-6715-81, Abcam ab188224), p53 (Genetex, GTX102965), γ-H2AX (Cell Signaling, 9718S), NRF2 (Proteintech, 16396-1-AP), NNT (Proteintech, 13442-2-AP), NAMPT (Proteintech, 66385-1-1g), TOM20 (Proteintech, 11802-1-AP), Citrate Synthase (Cell Signaling, 14039S), β-actin conjugated with HRP (Cell Signaling, 5125S), CD3e (Invitrogen, MA5-16622), Insulin (Abcam, ab181547), Cyp8b1 (Thermo, PA5-37088), and OXPHOS antibody cocktail (Abcam, ab110413) at 4 °C overnight. The membranes were incubated with HRP-conjugated secondary antibodies at room temperature for 2 hours and developed using Pico chemiluminescent substrate (Thermo Fisher Scientific) and imaged using an Amersham Imager 600 (GE Healthcare). At least three trials of Western blot analysis were performed.

### Bioinformatics and Statistical Analyses

scRNA-seq Gene Expression Analysis: Single-cell gene expression data were analyzed using Cell Ranger v5.0.0 (10× Genomics; support.10xgenomics.com/single-cell-gene-expression/software/pipelines/latest/what-is-cell-ranger). Briefly, raw BCL files from the Illumina HiSeq platform were demultiplexed to generate FASTQ files using ‘cellranger mkfastq’. Then, these raw FASTQ files were analyzed using ‘cellranger count’. The ‘cellranger count’ step includes mapping to the Mus musculus reference genome (mm10-2020-A), measuring gene expression with unique molecular identifiers (UMIs) and cell barcodes, determining cell clusters, and conducting differential gene expression analysis. The ‘count’ function can take input from multiple sequencing runs on the same library. The resulting raw count matrices were imported into Seurat 4.3.0. All genes expressed in ≥3 cells and all cells with at least 200 detected genes were used in downstream analysis. Rare subsets of cells with an abnormally high number of genes detected, suggesting potential multiplets, and low-quality or dying cells were filtered out if they had more than 6,000 genes or mitochondrial counts exceeding 15%. After filtering, the final count matrix contained 23,258 genes across 6,977 cells. Gene expression values were scaled and normalized for each gene across all integrated cells. Clustering and UMAP analysis were performed based on the statistically significant principal components. Significant top cluster markers for each cluster compared to all remaining cells were determined using the Wilcoxon rank sum test (min.pct=0.25, logfc.threshold=0.25, only the positive ones were filtered). Gene enrichment and functional annotation analysis for significant gene lists from two different results were performed using the g:Profilertool (biit.cs.ut.ee/gprofiler/).

GO and KEGG Analysis: GO and functional annotation analysis for significant probe lists were performed using KEGG (genome.jp/kegg/). All data analysis and visualization of differentially expressed genes was conducted using R 3.1.2 (r-project.org).

Network, STRING, Hierarchical Clustering, and Heatmap Analysis: Metabolomics data were further analyzed for network and pathway enrichment analyses were performed using MetaboAnalyst (version 5.0) (www.metaboanalyst.ca/). STRING analysis was performed using STRING (string-db.org/). Hierarchical clustering was conducted using the Ward variance minimization algorithm (method=‘Ward’) with Euclidean distance (metric=‘euclidean’) in SciPy (1.13.0). For heatmap analysis, the colors of individual cells in the heatmaps represent the Log 2 fold changes obtained using the 10× Genomics Loupe Browser v8.0.0. The values represent the ratio of normalized mean gene UMI counts of individual clusters compared to all other clusters. Genes with a smaller p-value are considered differentially expressed in the heatmap. P-values were adjusted using the Benjamini-Hochberg correction for multiple feature tests. Hierarchical clustering was performed using SciPy, applying the Ward variance minimization algorithm with Euclidean distance.

Cell Type Clustering Using Seurat: Calculation of single-cell expression matrices by Cell Ranger was performed using Seurat package for filtering, data normalization, dimensionality reduction, clustering, and gene differential expression analysis. The filtered scRNA-seq data were analyzed as follows:
1. Low-quality, multiplets, and mitochondrial contamination check: Cells with fewer than 200 genes, more than 8,000 genes, or a mitochondrial gene ratio greater than 25% were excluded. Doublets were detected using three standards: (1) The number of genes (nFeature), representing genes with at least one UMI count detected in each cell, which helps remove empty droplets or low-quality cells and identify doublets or multiplets. (2) The total number of molecules (nCount), representing the number of UMIs detected within a cell. (3) The percentage of reads mapped to the mitochondrial genome.
2. Aggregation for multiple datasets: Batch effects were corrected using anchors and canonical correlation analysis from the Seurat R library when aggregating multiple datasets.
3. Normalization and scaling: A global-scaling normalization method, “LogNormalize,” was applied to normalize gene expression measurements for each cell by total expression, multiply by a scale factor (10,000 by default), and log-transform the result.
4. Detection of variable genes: Highly variable genes showing a clear relationship between variability and average expression were extracted for dimensionality reduction analysis.
5. Scaling and centering data: The percentage of mitochondrial gene content was regressed against each feature, and the resulting residuals were scaled and centered. These residuals were used for dimensionality reduction and clustering analysis.
6. Principal component analysis (PCA): PCA was performed on the scaled data for graph-based clustering, selecting the number of principal components, k: 15.
7. Clustering cells: To find the graph-based clusters, f a K-nearest neighbor graph was first constructed based on the Euclidean distance in PCA space, and refined the edge weights between any two cells based on the shared overlap in their local neighborhoods (Jaccard similarity). Modularity optimization techniques such as the Louvain algorithm (default) to iteratively group cells together were also applied, with the goal of optimizing the standard modularity function. As UMAP/tSNE can help place cells with similar local neighborhoods in high dimensional space together into a low-dimensional space, UMAP was used for dimensionality reduction. UMAP is an algorithm for dimension reduction based on manifold learning techniques and ideas from topological data analysis.
8. Visualization: UMAP clustering results were visualized using the 10× Genomics Loupe Browser (version 8.0.0).

Following this pipeline, scRNA-seq data from 26,662 high-quality pancreatic tissue cells was processed to create a single-cell mouse pancreas atlas. 26 clusters representing senescence and diabetes-associated pancreatic islet cell types were identified.

Annotation of Cell Types Using CellKB: To confirm the classification of islet cell types, a three-step procedure was used: (1) SingleR, a reference-based scRNA-seq annotation, (2) comparison of cell numbers between groups, and (3) comparison of senescent beta-cell genes in each cluster. CellKB^{Pro} (version 2.1.2) (www.cellkb.com) is a knowledgebase of author-defined cell type marker gene sets (n=33,064) that can be rapidly searched for matching cell types (n=2,407), as previously reported. CellKB^{Pro} collects marker gene sets directly from publications describing primarily scRNA-seq and selected bulk RNA-seq or microarray experiments. It contains extensive cell type, tissue, and disease annotations for each cell type as described in the publication. To obtain a detailed and reliable pancreatic islet cell classification, DEGs of each cluster (in descending order of fold change) were uploaded. The query genes of each cluster were compared with every marker gene set in the database, and the best match was identified using a match score. The rank score was calculated based on the number of common genes between the query and the cell type, their ranks, their rank differences, and the total number of significant genes in the cell type. Higher match scores were assigned to cell types sharing highly ranked genes with the query. CellKB^{Pro} used an exponential function to score the matching genes, emphasizing higher-ranked matches more than lower-ranked ones. The algorithm also accounted for differences in gene list sizes between the query and various cell types to prevent disregarding cell types with fewer marker genes. After uploading the data, a list of relevant cell types (up to 100, ranked from highest to lowest score) were obtained for the DEGs of all 26 clusters. According to the rank-based score, 10 clusters (0, 2, 5, 10, 11, 12, 14, 20, 21, and 23) were classified as beta-cell clusters. Furthermore, among these beta-cell clusters, clusters 10, 11, 12, 14, 20, 21, and 23 exhibited a “senescent metaplastic cell” type. Thus, senescent beta-cell clusters were identified as 10, 14, 20, 21, and 23. Before confirming the expression levels of senescent genes in beta-cell clusters, cell numbers were compared between groups in each cluster. Clusters 10, 14, 20, 21, and 23 showed a consistent increase in cell numbers, while clusters 2, 5, 11, and 12 showed a lower cell proportion (%) in 30W-S compared to 12W-C or 30W-C groups. To reconfirm whether clusters filtered by steps 1 and 2 were indeed senescent beta-cell clusters, gene expression in each beta-cell cluster was compared with senescent beta-cell gene sets from previous studies characterizing senescent beta cells, using 69 genes (22 genes for hallmark beta-cell identity genes, 21 genes for beta-cell senescence, and 26 genes for beta-cell disallowed genes⁶):
A. Beta-cell identity genes: Rtx6, Pax6, Abcc8, Gck, Syt13, Vdz; Nkx2-2, Chga, Neurod1, Kcnj11, Ins1, Ins2, Mafa, Iapp, Nkx6-1, Pdx1, Dcx, Foxa2, Pklr; Elp4 (Sst and Lmo2 as negative controls),
B. Beta-cell senescence genes: Ulk3, Arntl, Igf1r; GIs, Mapk14, Cdkn1a, Trp53, Map2k1, Sirt1, Id2, 116, Cdkn2a, Il1a, Prkcd, Bambi, Plaur; Gls2, Cav1, Tbx2, Tbx3, Il1b.

C. Beta-cell disallowed genes: Fcgrt, Zcchc24, Arhgdib, Igf2bp2, Cox5a, Uqcrq, Zfp36l1, Pld2, Smad3, Cat, Selenbp1, Parp3, Zyx, Klhl5, Ldha, Arap2, Lmo4, Slc16a1, Tgm2, Yap1, Ndrg2, Mylk, Hpgds, Rnf144a, MgII, Rarres2.

These genes were assessed using Log 2 fold changes. Clusters 10, 14, 20, 21, and 23 were confirmed as senescent beta-cell clusters.

Analysis of Gene Expression in Publicly Available Datasets: Beta-cell RNA-seq data were obtained from human male and female datasets (E-MTAB-5061, GSE81608). SLC25A family genes, beta-cell identity genes, beta-cell disallowed genes, and beta-cell senescence genes were analyzed in each dataset. Additional pancreatic islet cell datasets (GSE73727, GSE85241, GSE87375) were analyzed for SLC25A51 mRNA levels. Non-pancreatic islet cell types were also analyzed using aging datasets from A. thaliana and S. cerevisiae (GSE43616, GSE210032) and human cell lines (WI38: GSE130727; MSC, IMR90 HUVEC: GSE98440; HDF161: GSE93535; RPE-1: GSE83647; LS8817: GSE74620; MRC5: GSE64553). The data were visualized using heatmaps or dot-plot graphs in GraphPad Prism.

Quantification and Statistical Analysis: Data are presented as mean±standard error of the mean (SEM). GraphPad Prism was used for two-tailed t-tests, one-way, or two-way ANOVA to assess significance between groups, followed by Bonferroni's multiple comparison test.

### Example 2. Analysis of Mitochondrial NAD+ Transporters from Various Model Organisms Undergoing Chronological Aging, as Well as Cells Undergoing Senescence In Vitro

To investigate the link between NAD+ compartmentalization, mitochondrial function, metabolic regulation, and aging, the expression of mitochondrial NAD+ transporters was analyzed using publicly available datasets from various model organisms undergoing chronological aging, as well as cells undergoing senescence in vitro. Mitochondrial NAD+ transport is mediated by Ndt1 and Ndt2 in yeast and plants. Recently, SLC25A51 was identified as the mitochondrial NAD+ transporter in mammals.

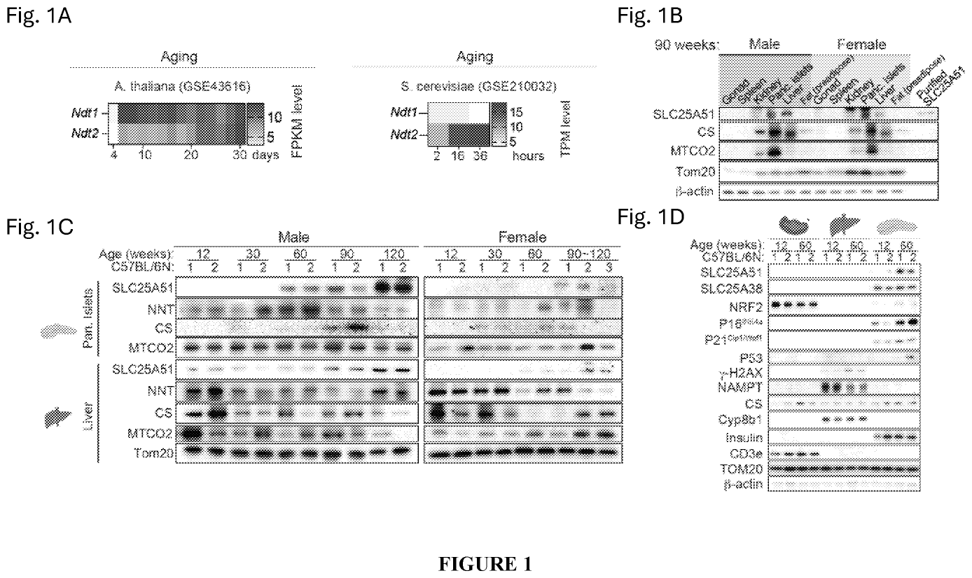

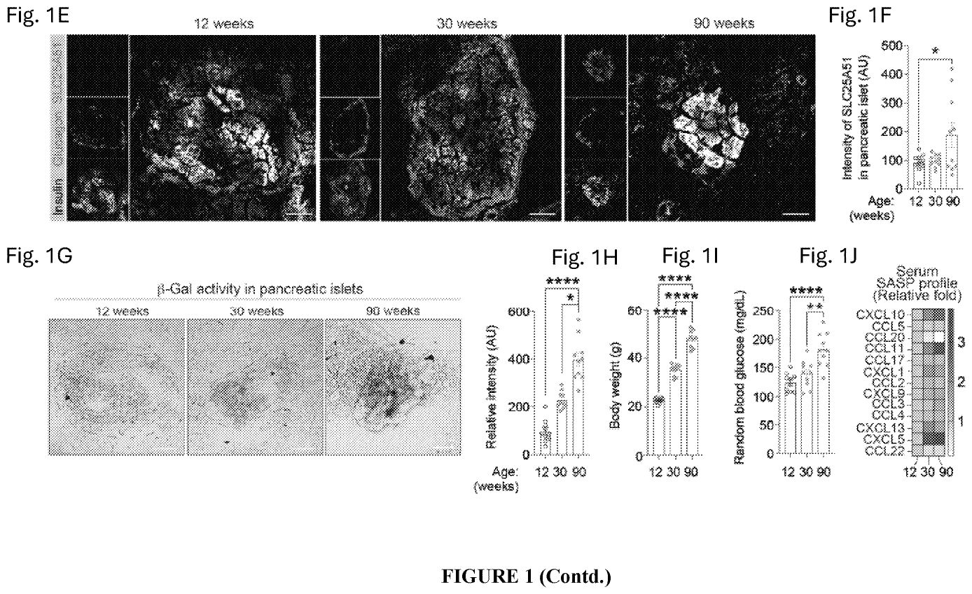

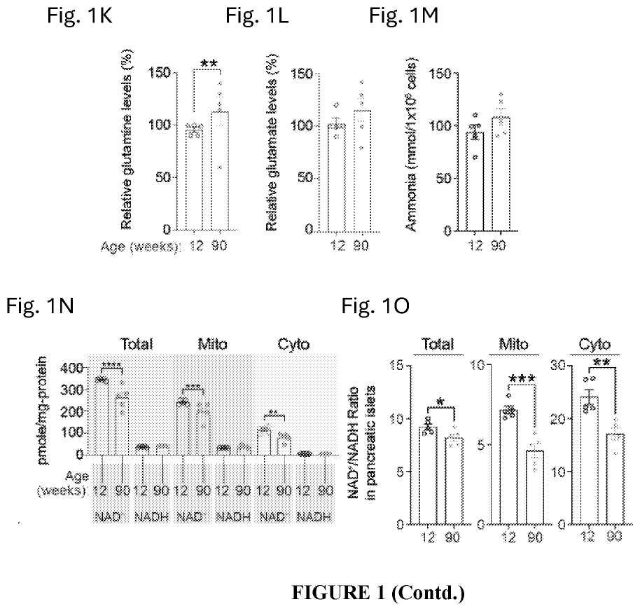

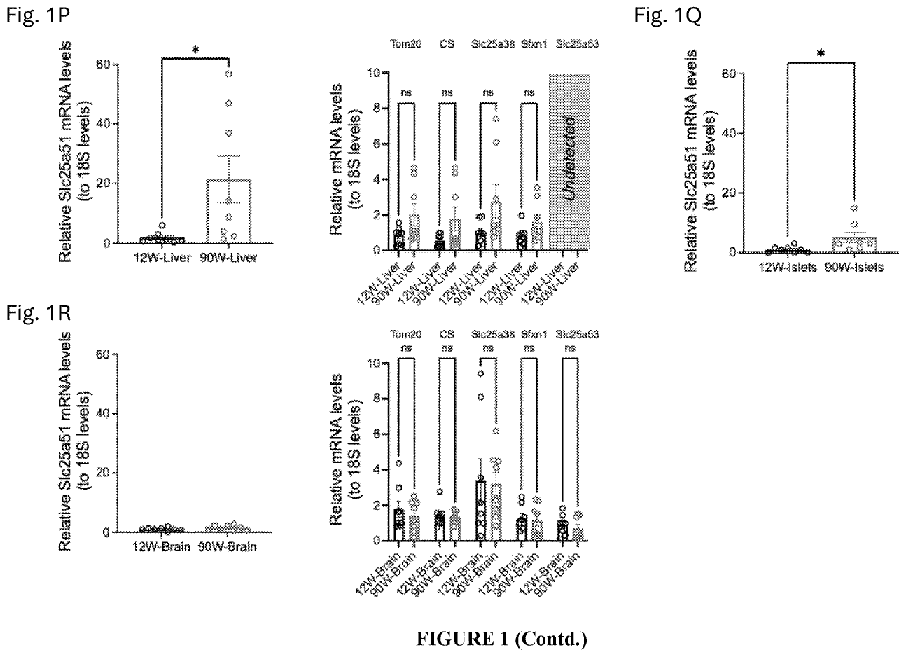

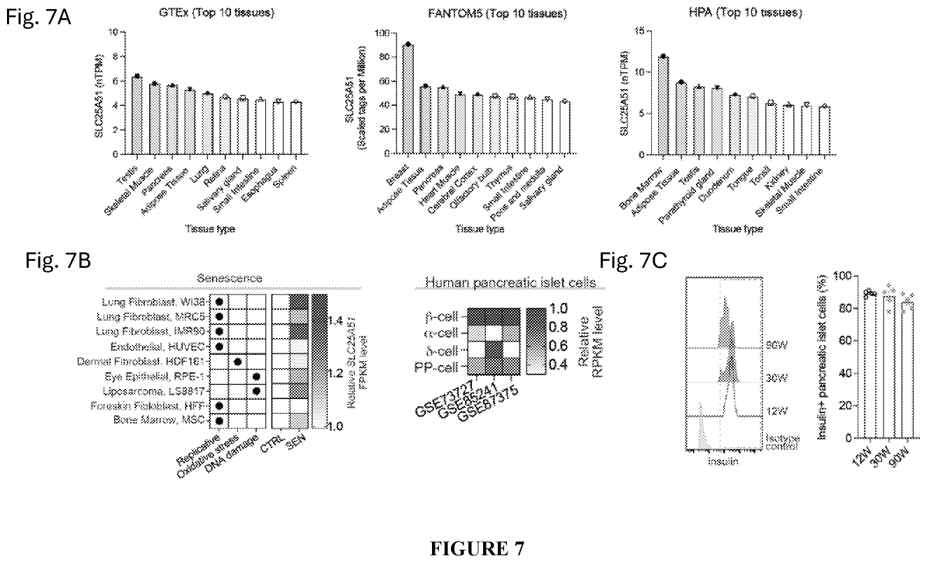

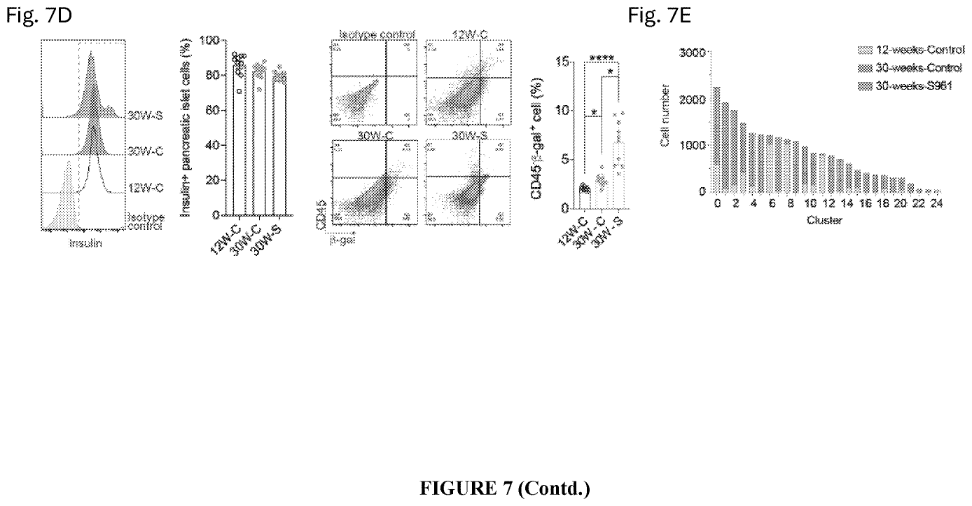

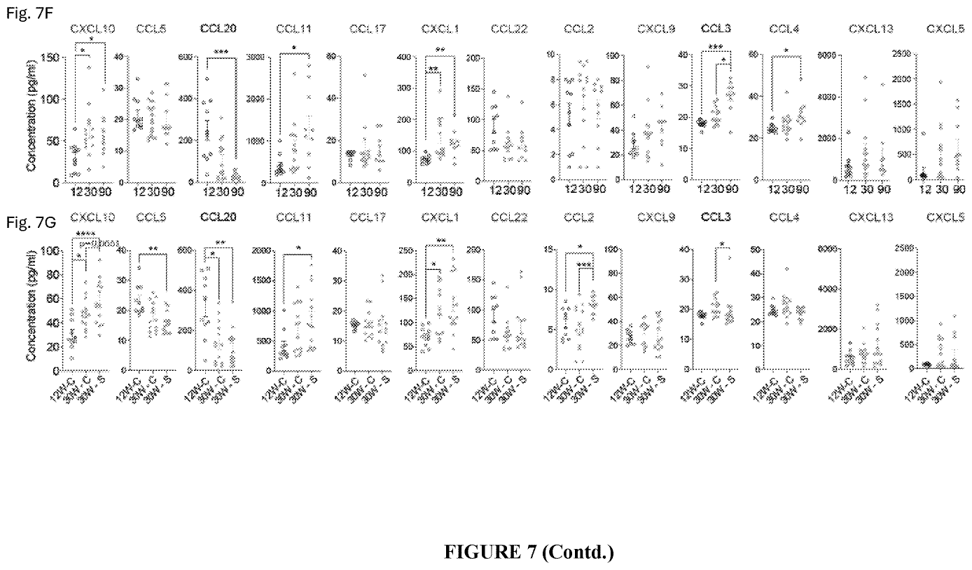

Expression of the SLC25A51 ortholog, Ndt2, was found to be upregulated in Arabidopsis thaliana and Saccharomyces cerevisiae during chronological aging (FIG. 1A). Furthermore, SLC25A51 was upregulated in several human cell lines undergoing replicative, oxidative stress-induced, or DNA damage-induced senescence (FIG. 7B). To validate these findings and identify tissues and cell types where SLC25A51 accumulates over time in mammals, SLC25A51 protein levels were examined in various isolated organs and tissues from 90-week-old male and female mice. SLC25A51 was ubiquitously expressed, with the highest mRNA levels observed in the testis, breast, pancreas, and adipose tissue (FIG. 7A). In aged mice, SLC25A51 protein was detected in pancreatic islets and the liver, but not in the gonads, kidney, spleen, or adipose tissue (FIG. 1B). Notably, SLC25A51 protein levels increased in pancreatic islets with age, particularly in male mice, whereas other mitochondrial proteins did not show a similar trend (FIGS. 1B-1D).

To detect changes in the expression of mitochondria-related genes with age, 12-week-old and 90-week-old C57BL/6N male mice (n=8 per group) were sacrificed. Liver tissues, pancreatic islets and brain tissues were collected to analyze the expression of mitochondria-related genes Slc25a51, Tom20, citrate synthase (CS), Slc25a38, Sfxn1 and Slc25a53. Expression levels of SLC25A51 were found to be significantly upregulated in the livers of 90-week old mice as compared to 12-week old mice, in contrast to Tom20, CS, Slc25a38, and Sfxn1 genes which showed no significant change in expression (FIG. 1P). Slc25a53 mRNA levels were not detected in liver tissues. Similarly, SLC25A51 expression was found to be significantly upregulated in pancreatic islets of 90-week old mice as compared to 12-week old mice (FIG. 1Q). However, SLC25A51 expression showed no significant differences in brain tissue of 90-week old mice as compared to 12-week old mice (FIG. 1R).

Mining a published scRNA-seq dataset from humans, it was found that SLC25A51 was predominantly expressed in beta cells, with minimal expression in other pancreatic islet cell types such as alpha cells, delta cells, or pancreatic polypeptide cells (FIG. 7B). Immunofluorescence (IF) staining of pancreatic tissues from aged mice confirmed that SLC25A51 accumulated in beta cells, the predominant cell type in mouse islets marked by insulin expression, but not in glucagon-producing alpha cells (FIGS. 1E, 1F).

In line with the increased prevalence of T2D and beta-cell senescence with aging, SLC25A51 accumulation in 90-week-old mice was accompanied by increased pancreatic beta-galactosidase (beta-Gal) activity, p16^{INK4A} and p21^{Cip1/Waf1} levels (markers of senescent beta cells, body mass, random blood glucose levels, and serum secretory associated phenotype (SASP) factors, such as CXCL10 (FIGS. 1G, H-J, 7F).

Thus, the analysis demonstrates that induction of mitochondrial NAD+ transporters is an evolutionarily conserved feature of chronological aging.

### Example 3. Analysis of Mitochondrial NAD+ Levels and SLC25A51 Expression in Pancreatic Beta Cells with Aging

At the whole-tissue level, NAD+ levels decline with aging in the pancreas and other organs. Given the increased expression of the mitochondrial NAD+ transporter SLC25A51 in pancreatic beta cells with aging, whether mitochondrial NAD+ levels in beta cells changed concomitantly was assessed.

Pancreatic islet cells isolated from 12-week, 30-week, and 90-week-old mice were found to consist of 80% insulin-producing beta cells (FIG. 7C). Compared to 12-week-old mice, 90-week-old mice exhibited a decline in total NAD+ level, as well as the NAD+/NADH ratio, in whole-cell lysates (total), cytosolic fractions, and mitochondrial fractions (FIGS. 1N, 1O). Notably, this decline occurred despite the increased expression of SLC25A51. Other metabolic features characteristic of senescent cells and tissues were observed, including increased glutamine, glutamate, and cellular ammonia levels in whole-cell lysates, indicative of enhanced glutaminolysis (FIGS. 1K-1M).

These findings indicate that both whole-tissue and mitochondrial NAD+ levels decrease in pancreatic islets during chronological aging, in contrast to the increase in SLC25A51 expression.

### Example 4. Investigation of SLC25A51 Expression and Function In Vivo

To investigate SLC25A51 expression and function and to test whether SLC25A51 accumulates in beta cells as they become senescent, contributing to metabolic changes and the senescence phenotype, scRNA-seq was performed. A mouse model of T2D and acute beta-cell senescence induced by the insulin receptor antagonist S961 was used (Aguayo-Mazzucato et al, Cell Metab (2017) 25, 898-910 e895).

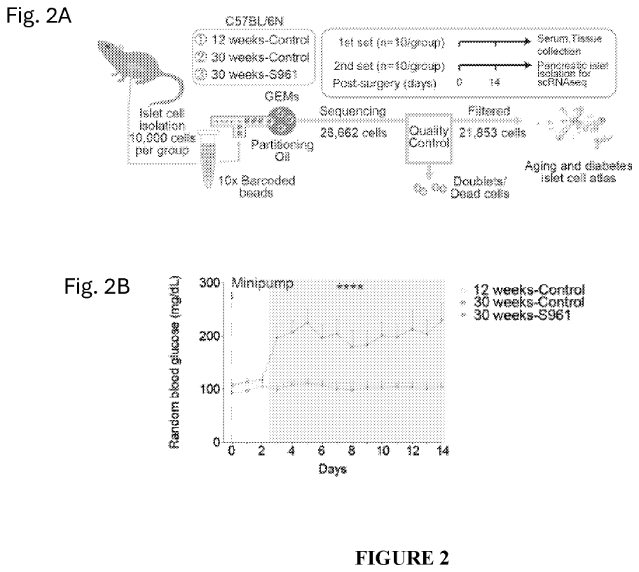

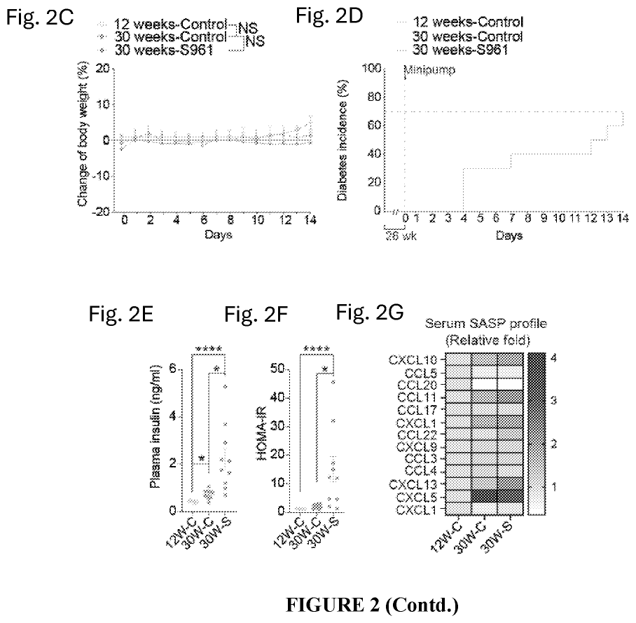

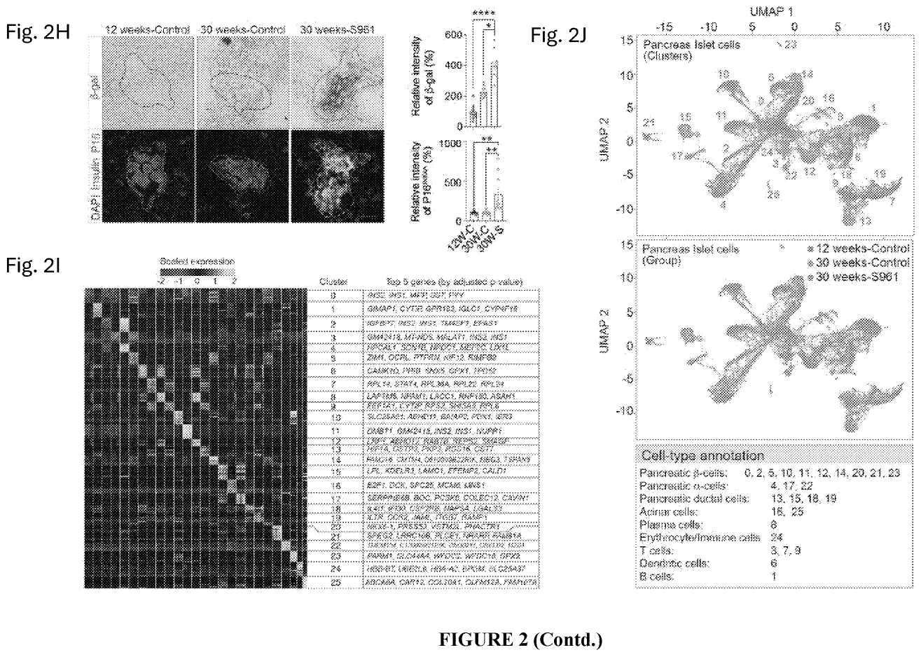

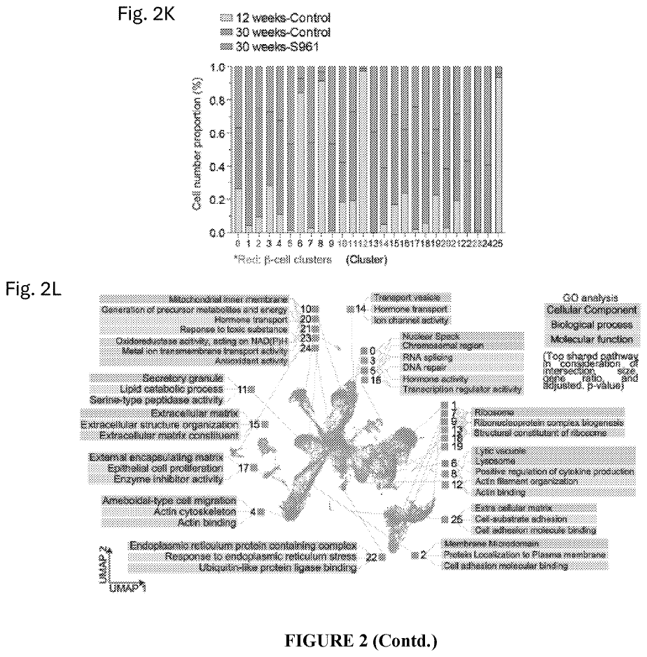

Systemic treatment was administered with S961 or PBS to C57BL/6N mice at 28 weeks of age (n=10/group) using an osmotic pump for two weeks (FIG. 2A).

S961 treatment induced hyperglycemia (12-week-old control [12W-C]: 111.20±12.04 mg/dL; 30-week-old control [30W-C]: 104.20±14.69 mg/dL; 30-week-old S961 treated [30W-S]: 229.70±98.21 mg/dL) and hyperinsulinemia (12W-C: 6.30±0.04 ng/ml; 30W-C: 15.91±0.19 ng/ml; 30W-S: 25.80±1.39 ng/ml) compared to both 12- and 30-week-old control mice (FIGS. 2B-2F). Overall, diabetes incidence was increased in S961-treated mice compared to control mice at 30 weeks (70% versus 0%, n=10/group) (FIG. 2D). These effects were independent of body weight (FIG. 2C).

To assess beta-cell senescence, SASP production, beta-Gal activity, and p16^{INK4A} levels were examined in pancreatic beta cells. SASP-related chemokines, such as CXCL10, CCL4, CCL3, CXCL1, and CCL5, have previously been reported to increase in hydrogen peroxide (H₂O₂)-induced senescent MIN6 beta cells. Furthermore, human and mouse senescent beta cells secrete SASP factors such as eNAMPT, CCL2, CCL4, CXCL2, and CXCL3. In addition, CCL20, CCL11, CCL17, CCL22, CXCL9, CXCL13, and CXCL5 are critical SASP factors in different tissue types and disease models, although they have not been evaluated in S961-treated diabetic mouse models. It was observed that CCL2 was elevated in the 30W-S group compared to both the 12W-C (p=0.0008) and 30W-C (p=0.0165) groups, while CXCL10 was slightly elevated, reaching significance only compared to 12-week-old control animals (12W-C: p<0.0001; 30W-C: p=0.0651). Other SASP factors did not show significant differences between the 30W-C and 30W-S groups (FIGS. 2G, 7G). Staining for CD45, a pan-lymphocyte marker, and beta-Gal activity in pancreatic islet cells revealed a 2- and 4-fold increase in CD45^{Negative} beta-Gal^{positive} beta cells in the 30W-S group compared to the 30W-C and 12W-C groups, respectively (FIG. 7D). Staining of pancreatic tissue confirmed increased beta-Gal activity and p16^{INK4A} expression in the 30W-S group in insulin^{positive} pancreatic beta cells (FIG. 2H).

In summary, these data indicate that systemic S961 treatment in C57BL/6N mice induces beta-cell senescence by increasing Igf1r and p16′ mRNA levels and insulin levels, contributing to the development of insulin resistance and T2D.

To specifically identify and profile senescent beta-cell subsets, single-cell atlases were generated for pancreatic islets isolated from three groups: 12W-C, 30W-C, and 30W-S mice (FIGS. 2A, 2I, 2J). To define each cell type, the sequencing data was processed using the Seurat R package for quality control, normalization, batch effect correction, and clustering. Each cell type was annotated based on the expression levels of canonical cell-type-specific markers. In total, 26 clusters were identified across the three groups, each exhibiting differentially expressed genes (DEGs) (FIG. 2I). These clusters were annotated as ten pancreatic beta-cell subsets, three alpha-cell subsets, four ductal cell subsets, two acinar cell subsets, and three T-cell subsets (FIG. 2J). Additionally, single clusters corresponding to plasma cells, erythrocyte/immune cells, dendritic cells, and a B-cell population were identified (FIG. 2J). All ten beta-cell clusters were classified as independent cell types based on their distinct DEGs (FIG. 2J). Using the CellKb^{pro} ranking-based score program, all 26 clusters were annotated, identifying clusters 10, 14, 20, 21, and 23 as “senescent metaplastic cells” as well as “beta cells” (FIG. 1J). Four of the five putative senescent beta-cell clusters (10, 14, 20, and 23) had a higher cell number and proportion (%) in the 30W-S group compared to the 12W-C or 30W-C groups (FIGS. 2K, 7E). Gene Ontology (GO) and pathway analyses revealed that clusters 10, 20, 21, and 23 were predominantly enriched in GO terms related to the mitochondrial inner membrane, while cluster 14 was enriched in transport vesicle (FIG. 2L).

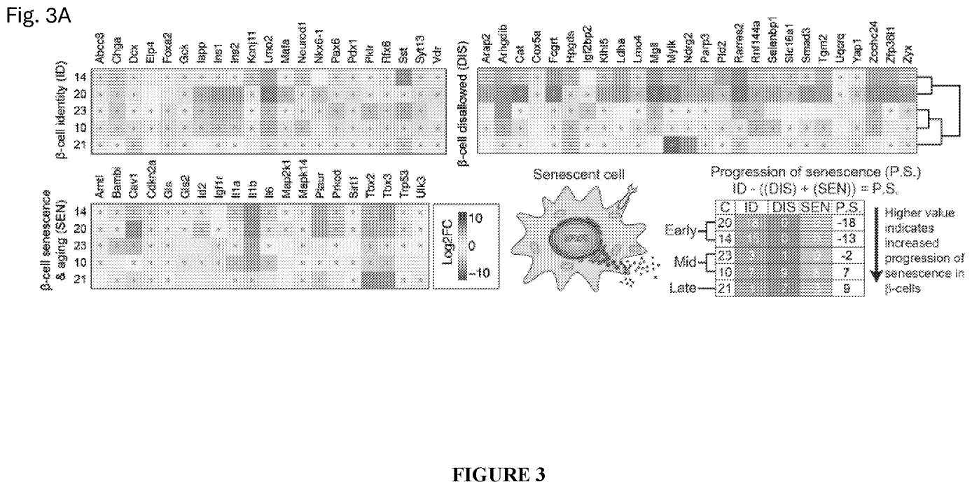

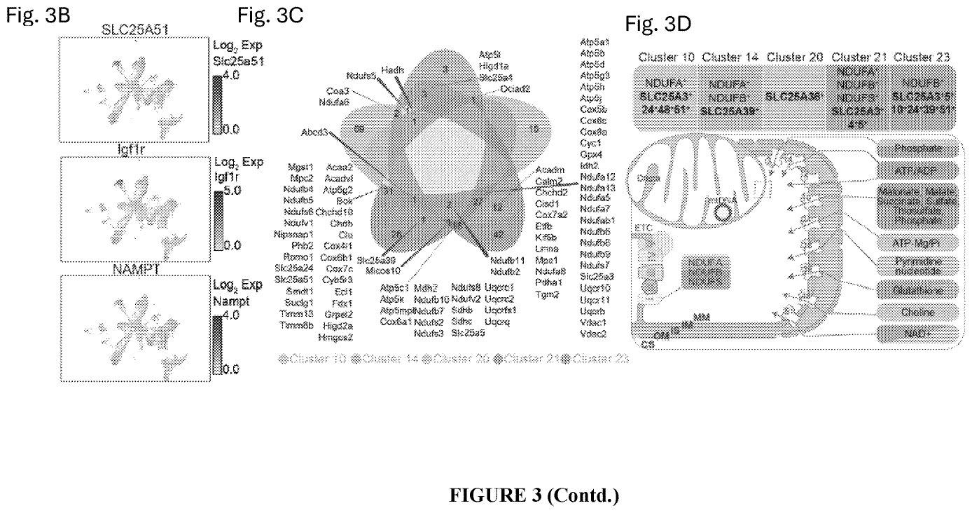

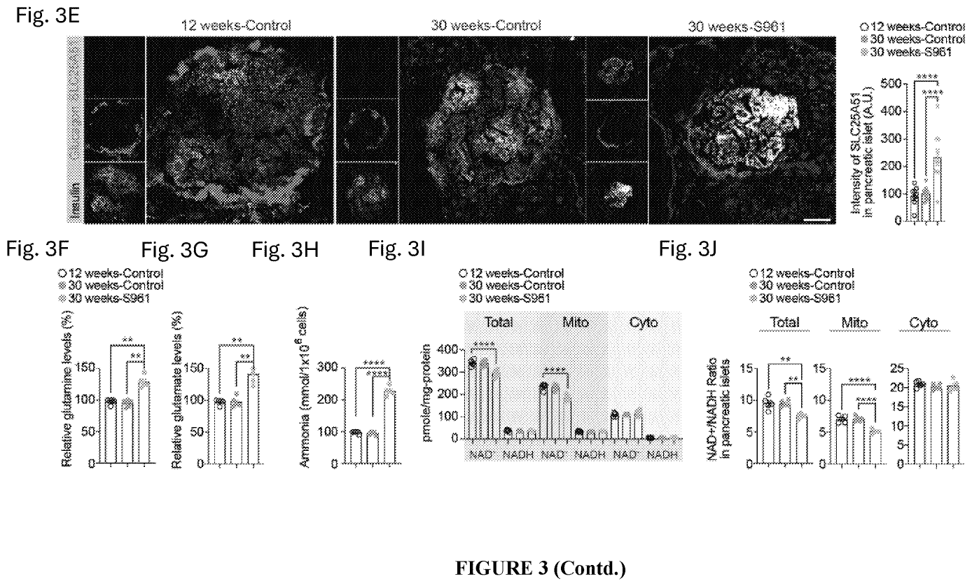

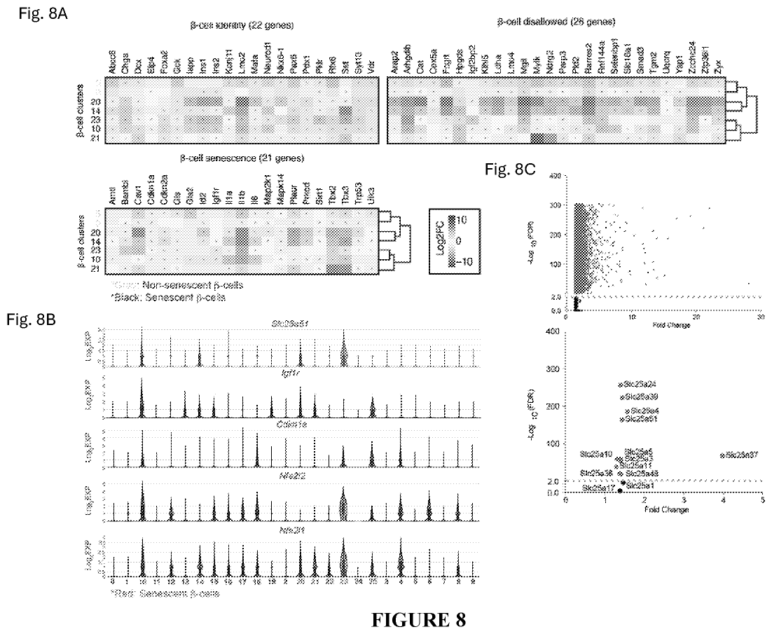

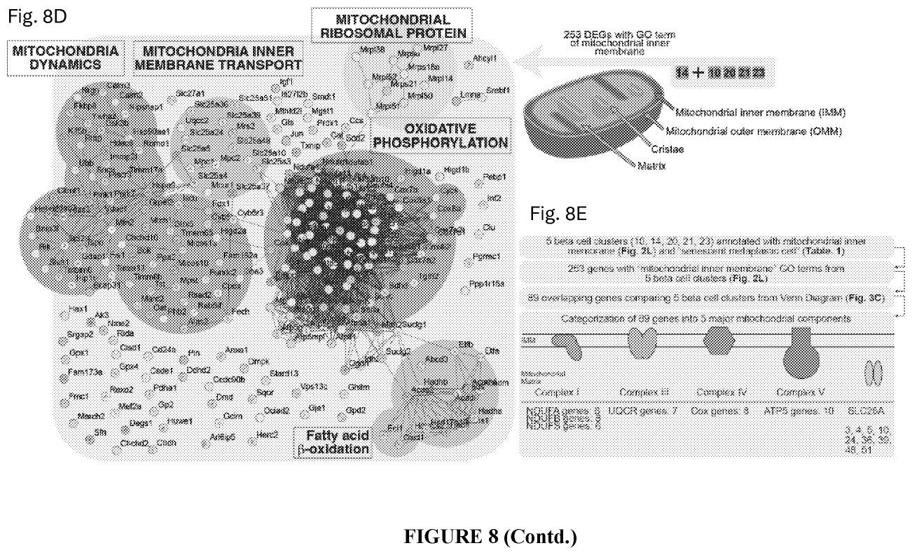

To further confirm whether clusters 10, 14, 20, 21, and 23 represent senescent beta cells, each cluster was analyzed for senescent beta-cell characteristics by comparing the expression of three beta-cell indices: beta-cell identity-related genes, beta-cell disallowed genes, and beta-cell senescence (and aging)-related genes. Research has established that senescent beta cells lose expression of beta-cell identity-related genes while exhibiting increased expression of beta-cell disallowed genes and senescence- and aging-associated genes. It was observed that the different senescent beta-cell subsets displayed heterogeneous gene expression levels across the three indices (FIGS. 3A, 8A). To more broadly categorize each cluster into different senescence stages, a “Progression of Senescence (P.S.)” equation was developed to numerically capture and integrate the diverse genetic markers associated with senescence. Consistent with the dendrogram analysis (FIG. 3A), the cell clusters were grouped into three stages using the P.S. equation-early, mid, or late. These results suggest that the heterogeneity of senescent beta-cell clusters may reflect “snapshots” of different stages of the senescence process.

To identify commonly shared genes among the different stages of beta-cell senescence, a Venn diagram analysis was performed across five senescent beta-cell clusters. No single DEG was identified as a shared gene across all clusters (FIG. 3C). However, further analysis identified 89 genes shared between a few clusters, primarily associated with five mitochondrial components: Complex I, Complex III, Complex IV, Complex V, and SLC25A family genes (FIGS. 3C, 3D, 8B-8E). Using these mitochondrial component genes, particularly mitochondrial inner membrane transporter SLC25A family genes, it was possible to distinguish each cluster: Cluster 10 (SLC25A3⁺24⁺48⁺51⁺), Cluster 14 (SLC25A39⁺), Cluster 20 (SLC25A36⁺), Cluster 21 (SLC25A3⁺4⁺5⁺), and Cluster 23 (SLC25A3⁺5⁺10⁺24⁺39⁺51⁺) (FIGS. 3D, 8C, 8G). Thus, pancreatic islet cells isolated from S961-treated C57BL/6N mice are transcriptionally heterogeneous and exhibit increased senescent beta-cell features marked by mitochondrial inner membrane transporter gene signatures.

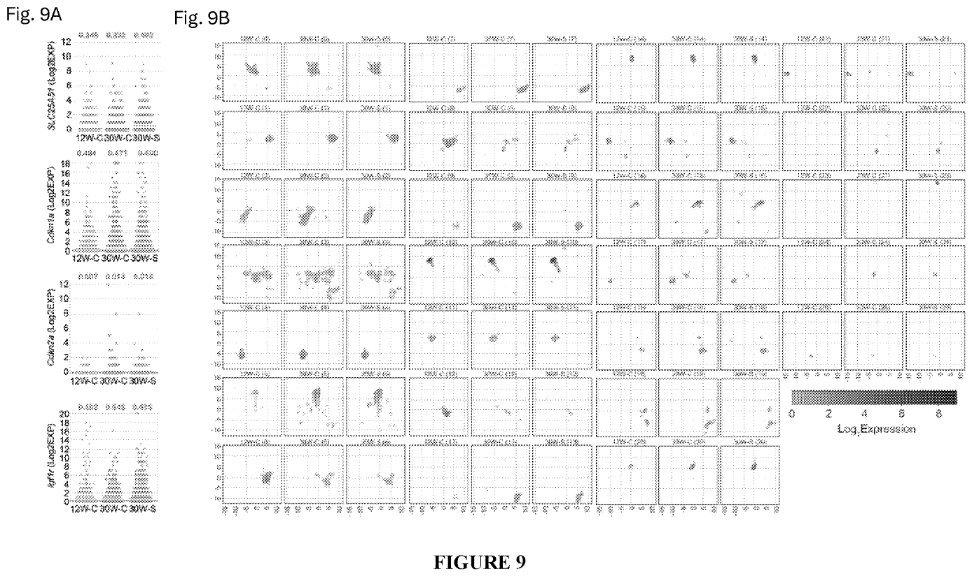

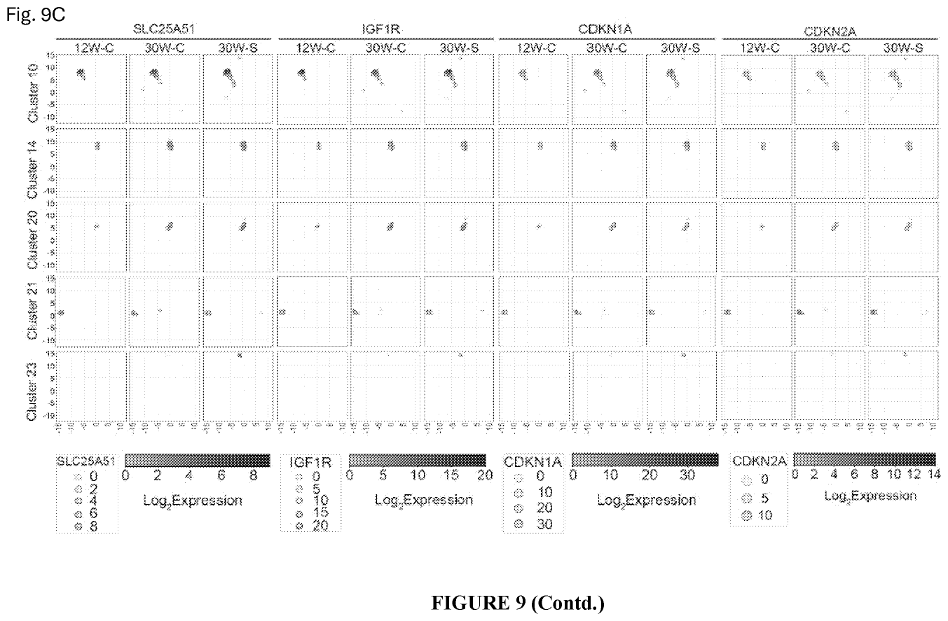

Notably, among the SLC25A genes, the mitochondrial NAD⁺-transporter SLC25A51 was highly expressed in clusters 10, 14, 20, and 23 (FIGS. 3B, 8B, 9A-9C) and served as a DEG in the ‘mid-stage’ of beta-cell senescence, represented by clusters 10 and 23 (FIG. 3A, 3D).

Overall, SLC25A51 was the sole gene identified as a DEG in more than two senescent beta-cell clusters, and its expression correlated with the beta-cell aging and senescence markers IGF1R and NAMPT, respectively (FIGS. 3B, 3C, 8B).

### Example 5. Analysis of SLC25A51 Protein Levels in Beta Cells in the S961 Model

To confirm whether SLC25A51 protein levels were concomitantly increased in beta cells in the S961-treated mice, IF staining was performed in pancreatic tissues from experimental cohorts.

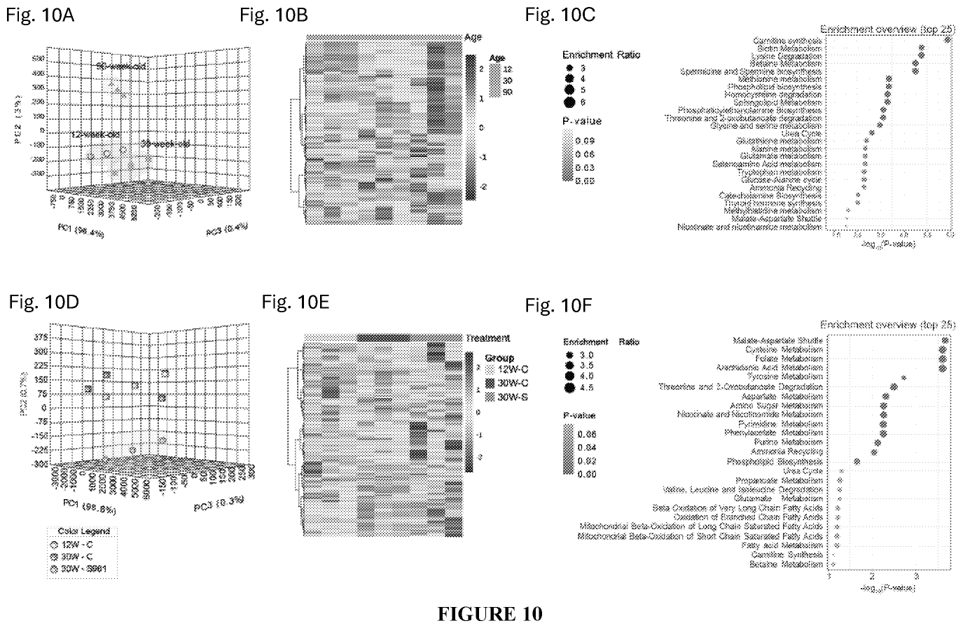

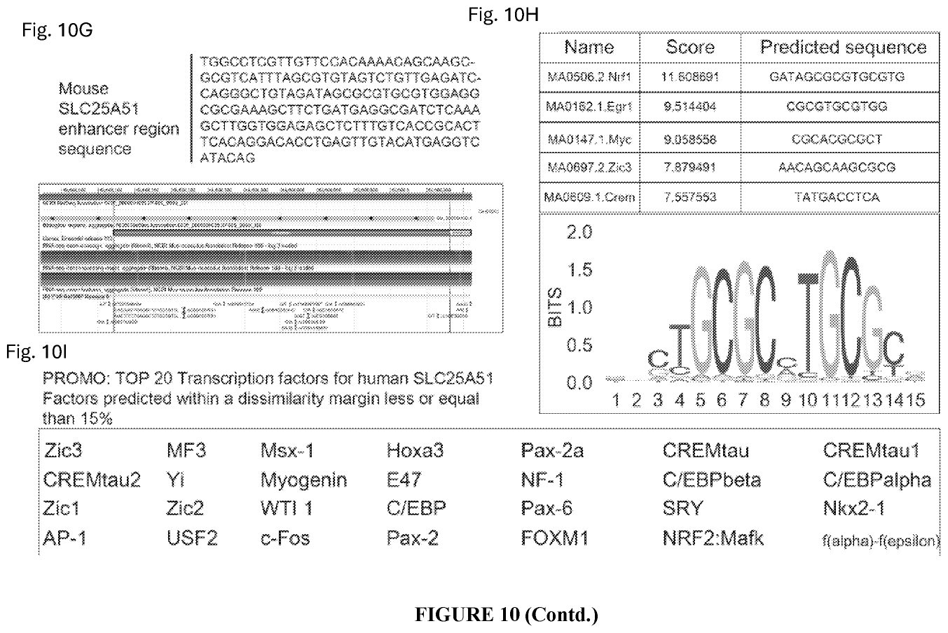

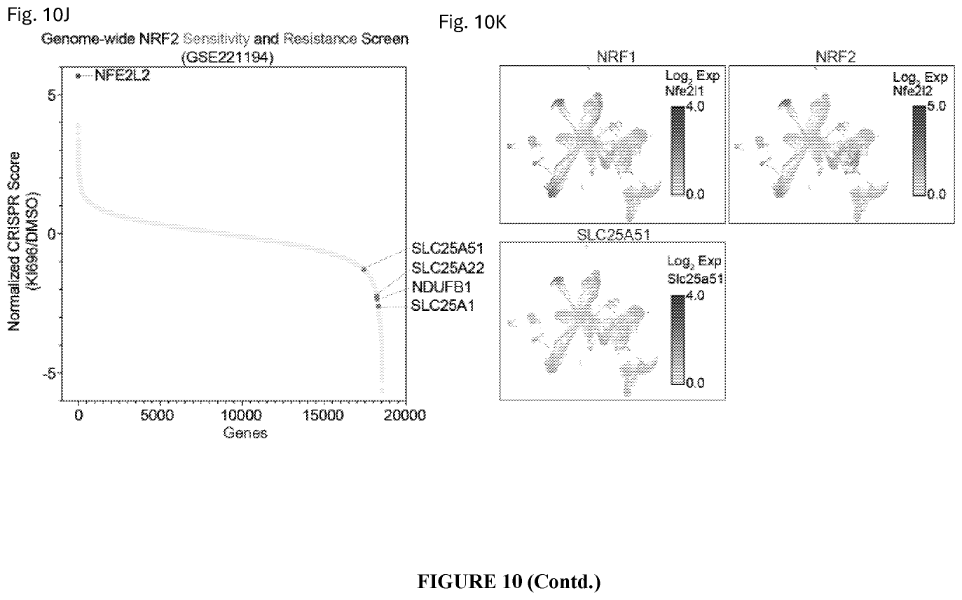

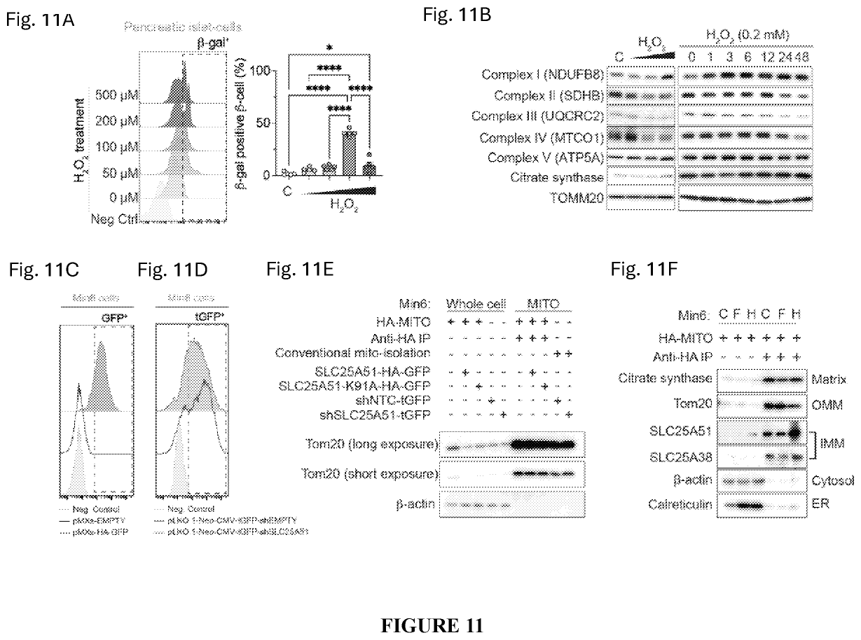

SLC25A51 accumulated in beta cells isolated from S961-treated animals compared to control groups (FIG. 3E), consistent with the observation in the chronological aging model (FIG. 1E). To gain a metabolic overview in these mice, serum was collected for targeted metabolomic analysis from both chronologically aged and S961-treated mice. Interestingly, metabolites in pathways influencing NAD⁺ levels or redox balance, including the malate-aspartate shuttle and nicotinate and nicotinamide metabolism, were significantly altered between groups (FIGS. 10A-10F). To further understand how these metabolites are altered at the sub-cellular level, pancreatic islets were isolated either from chronologically aged (FIGS. 1N, 1O) and S961-treated mouse models (FIGS. 31, 3J). In chronologically aged mouse model, a decreased NAD⁺/NADH ratio was observed in whole-cell lysates, cytosol, and mitochondria isolated from pancreatic islets (FIGS. 1N, 1O). However, mitochondrial, cytosolic, and whole-cell NAD⁺ and NADH levels were lower in the S961-treated group, with the NAD⁺/NADH ratio also reduced in the mitochondria and whole-cell lysates, but not in the cytosol, compared to the 12-week-old and 30-week-old untreated control groups (FIGS. 31, 3J). Similar to the metabolic changes observed in chronological aging (FIGS. 1K-1M), S961-treated mice showed increased glutamine, glutamate, and cellular ammonia levels compared to control groups, indicative of potential changes in glutaminolysis (FIGS. 3F-3H).

Glutaminolysis is a metabolic pathway essential for cell survival during senescence, and SLC25A51 is required for glutaminolysis and the entry of glutamate into the TCA cycle. Thus, upregulation of SLC25A51 in pancreatic islets of S961-treated or chronologically aged mice might be an adaptation or a driver of the metabolic changes in beta cells undergoing senescence.

Overall, SLC25A51 is part of a senescence gene signature in pancreatic beta cells.

### Example 6. In Vitro Evaluation of the Connection Between SLC25A51 and Senescence in Beta Cells

To mechanistically probe the functional connection between SLC25A51 and senescence in beta cells, in vitro studies were conducted using both murine pancreatic islets and MIN6 mouse insulinoma cells. While SV40 T antigen-immortalized MIN6 cells do not undergo a senescence-associated cell cycle arrest, they recapitulate aspects of senescence, including increased beta-Gal activity and the induction of senescence-associated factors (p16^{INK4A}, p21^{Cip1/Waf1}, NAMPT) and SASP factors in response to prolonged passaging or H₂O₂ treatment.

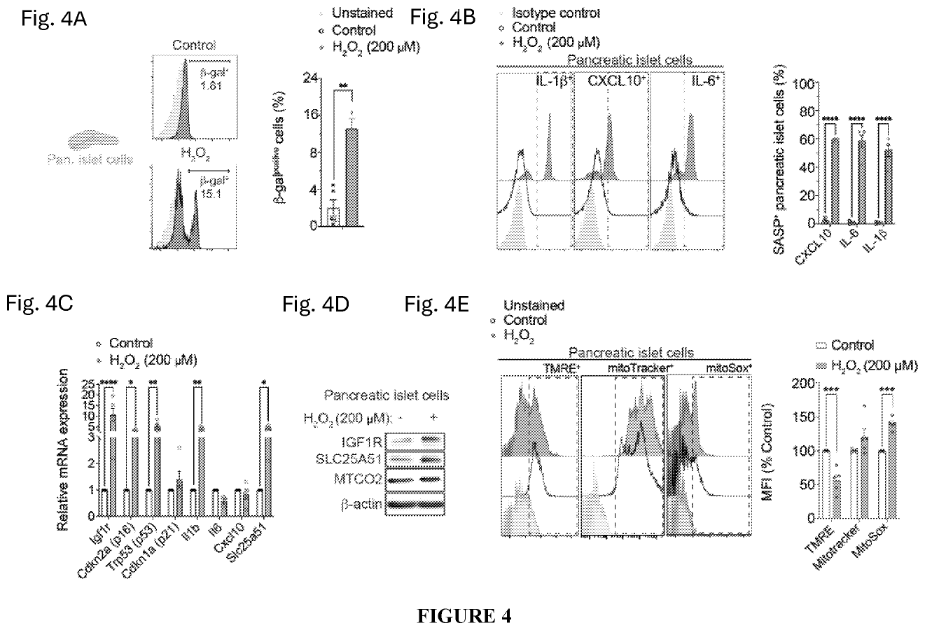

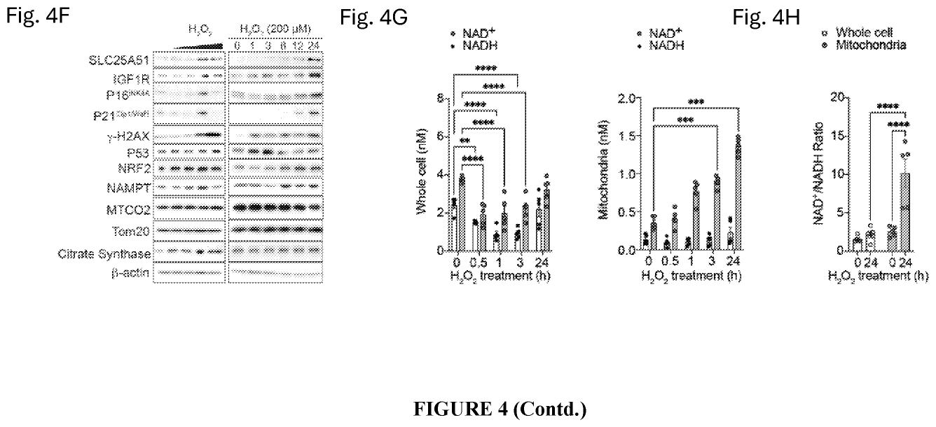

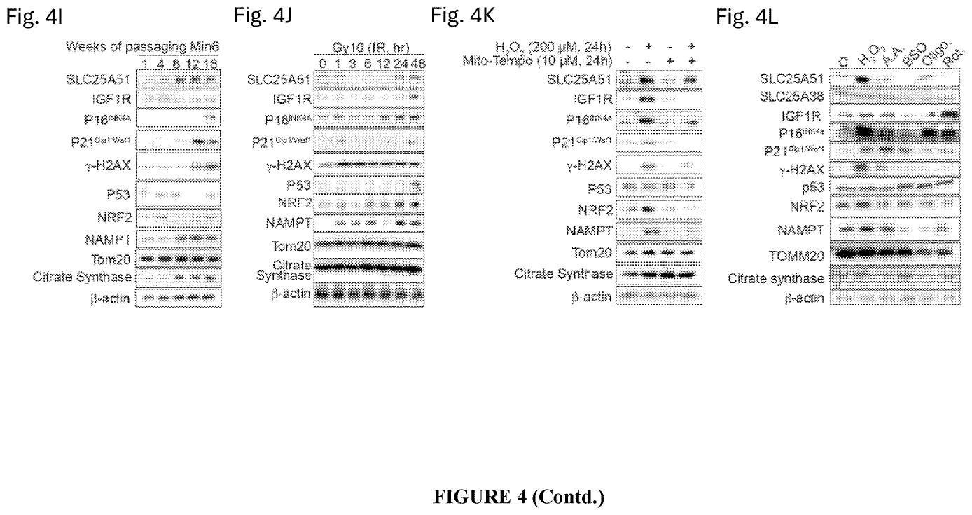

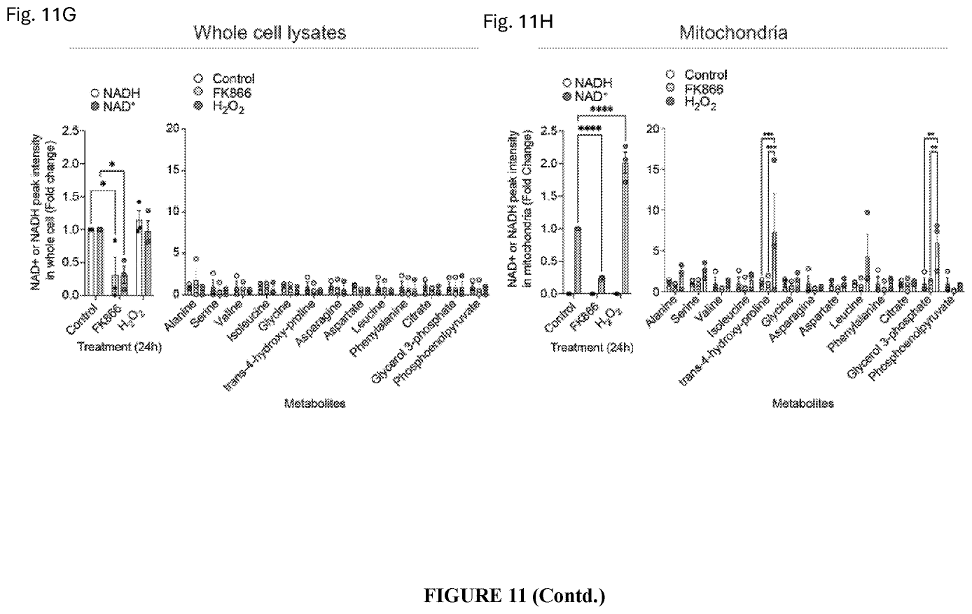

Primary pancreatic islet cells isolated from 30-week-old C57BL/6N mice (n=5/group) exhibited increased beta-Gal activity in a concentration-dependent manner following H₂O₂ treatment, peaking at 200 μM, with diminished activity and increased cell death at concentrations exceeding 500 μM (FIGS. 4A, 11A, data not shown). Thus, mRNA and protein levels of senescence markers were evaluated 24 hours after treatment with 200 μM H₂O₂. H₂O₂ treatment elevated SASP production (CXCL10, IL-6, and IL-1β) and increased mRNA levels of Slc25a51 and the beta-cell senescence markers Igf1r, Cdkn2a, Trp53, and Il-1b (FIGS. 4A-4C, 11A). Additionally, IGF1R and SLC25A51 protein levels were increased in pancreatic islet cells following H₂O₂ treatment (FIG. 4D). Similar to pancreatic islet cells, SLC25A51 and senescence markers (p16^{INK4A}, p21^{Cip1/Waf1}, γ-H2AX, NAMPT) were upregulated in a concentration- and time-dependent manner in MIN6 cells, with maximal levels observed at 200 μM for 24 hours (FIG. 4F). Treatment with MitoTempo, a mitochondria-localized redox modulator that removes reactive oxygen species (ROS) and reduces diabetic complications⁷⁰, prevented the H₂O₂-induced upregulation of SLC25A51 and senescence markers, including p16^{INK4A}, p21^{Cip1/Waf1}, NAMPT, and γ-H2AX (FIG. 4K). Oxidative stress induced by H₂O₂ treatment not only triggers senescence but also impairs glucose-induced insulin secretion in pancreatic beta cells by depolarizing the mitochondrial membrane potential. Indeed, TMRE and mitoSOX staining revealed that H₂O₂ decreased mitochondrial membrane potential and increased mitochondrial superoxide, respectively (FIG. 4E), without altering mitochondrial mass as indicated by MitoTracker staining (FIG. 4E). While citrate synthase and Complex I proteins increased following H₂O₂ treatment, MTCO2 and TOM20 levels remained unchanged (FIGS. 4F, 11B).

Because H₂O₂ treatment directly impacts mitochondrial physiology, it was further investigated whether the mitochondrial NAD⁺ transporter SLC25A51 was influenced by replicative senescence or irradiation with 10 Gy of ionizing radiation (IR). Similar to H₂O₂ treatment, both prolonged passaging and irradiation of MIN6 cells increased SLC25A51 levels and senescence markers such as p16^{INK4A}, p21^{Cip1/Waf1}, and γ-H2AX (FIGS. 41, 4J). These results indicated that SLC25A51 accumulation occurs in response to beta-cell senescence in a beta-cell-specific, oxidative stress/ROS-dependent manner. SLC25A51 is the major mitochondrial NAD⁺ transporter in mammals, and it is conceivable that its upregulation is a consequence of the drop in whole-cell or mitochondrial NAD⁺ levels or the NAD⁺/NADH redox ratio in pancreatic islets that have been observed during aging and senescence (FIGS. 1O, 1P, 3I, 3J).

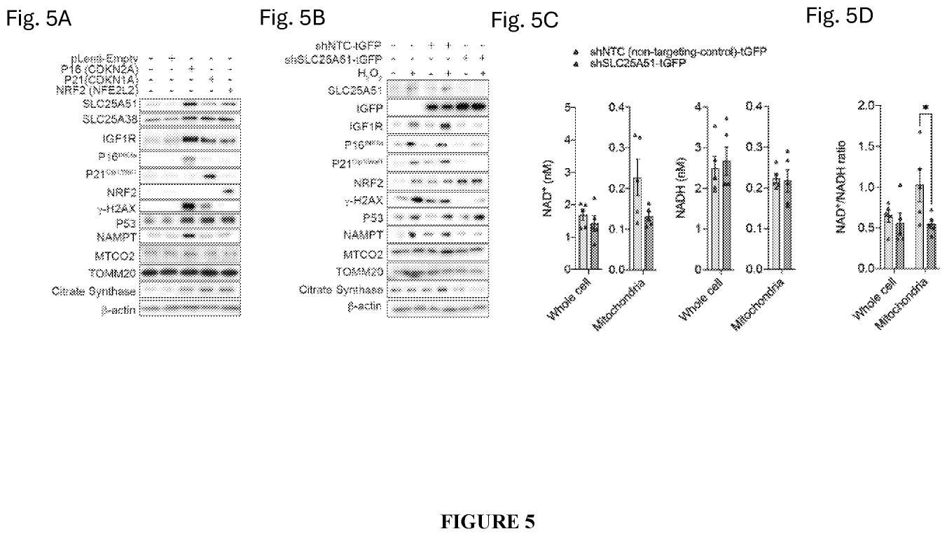

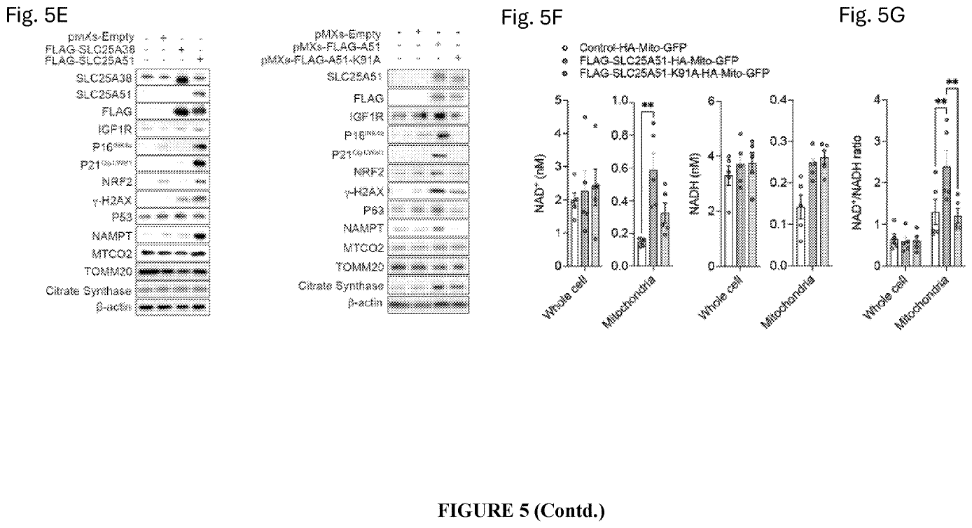

In MIN6 cells cultured in media containing excess concentrations of NAD⁺ precursors, such as nicotinamide, a decline in total cellular NAD⁺ levels was observed during H₂O₂-induced senescence from 0.5 to 3 hours, followed by a re-stabilization of NAD⁺ levels at 24 hours (FIG. 4G). The decrease in total cellular NAD⁺ levels and increased oxidative stress from H₂O₂ at early time points led to the induction of NRF2, a master regulator of the oxidative stress response, between 6 and 24 hours (FIG. 4F). NRF2 activation can upregulate the NAD⁺-consuming enzyme ALDH3A1 and decrease the NAD⁺/NADH ratio in lung cancer cells⁷⁴. Whether NRF2 can directly regulate Slc25a51 has not been evaluated. In an scRNA-seq analysis, it was observed that the Nfe2l2 (NRF2) and Nfe2l1 (NRF1) genes were upregulated in clusters with high Slc25a51 expression levels (FIGS. 8B, 10K). Furthermore, transcription factor prediction programs (JASPAR and PROMO) identified Nfe2l1 (NRF1) and Nfe2l2 (NRF2) as candidates for regulating Slc25a51 expression (FIGS. 10G-10I). By analyzing published data from a genome-wide CRISPR screen in KEAP1-NRF2 interaction inhibitor (K1696)-treated KEAP-1-dependent CALU6 cells⁷⁴, Slc25a51 was found to be one of the genes sensitive to NRF2 activation (FIG. 10J). Furthermore, H₂O₂-treatment, irradiation, and overexpression of Nfe2l2 in MIN6 cells led to an increase in SLC25A51 levels (FIGS. 4F, 4J, 5A).

These data implicate NRF2 as a potential regulator of SLC25A51 expression in beta cells, suggesting a mechanism to induce mitochondrial NAD⁺ influx and metabolic adaptation in response to oxidative stress.

To assess whether H₂O₂ treatment indeed induces mitochondrial NAD⁺ influx, mitochondria were isolated to measure NAD⁺ and NADH levels. Unlike total NAD⁺ levels, H₂O₂ treatment increased mitochondrial NAD⁺ levels ~4-fold after 24 hours and the NAD⁺/NADH redox ratio ~3-fold, concurrent with an increase in SLC25A51 protein levels, while whole-cell NAD⁺ and NADH levels remained unchanged (FIGS. 4G, 4H, 10G-10H). In contrast, treatment with the Complex I inhibitor, Rotenone (Rot), or other electron transport chain (ETC) inhibitors (Antimycin A [A.A] and Oligomycin A [Oligo]) did not induce SLC25A51 or NRF2 expression (FIGS. 4L, 10B). Induction of oxidative stress by treating cells with the glutathione synthesis inhibitor BSO increased senescence markers, such as p16^{INK4A} and p21^{Cip1/Waf1}, but failed to induce SLC25A51 or Nrf2 (FIG. 4L). Moreover, reducing whole-cell and mitochondrial NAD⁺ by inhibiting the NAD⁺ salvage enzyme NAMPT with FK866 did not induce SLC25A51 expression (FIGS. 10F-10H).

Thus, SLC25A51 is upregulated in senescence downstream of p16^{INK4A} and potentially NRF2, and modulates intracellular NAD+ compartmentalization to contribute to the senescence phenotype. Overall, the data indicate that SLC25A51 upregulation does not occur as a compensatory response to lower cellular or mitochondrial NAD⁺ levels, changes in the NAD⁺/NADH redox ratio, or mitochondrial dysfunction. Instead, it may serve as part of the senescence response to drive metabolic changes, such as increased glutaminolysis, that are necessary to sustain the senescent cell state.

### Example 7. Analysis of SLC25A51 Induction During the Senescence Program

While senescence can be induced downstream of DNA damage, metabolic or mitochondrial changes such as impaired glucose sensing and insulin production in beta cells, and mitochondrial insults in different senescent cell types, are central steps in the senescence program. To determine whether SLC25A51 induction occurred as part of the senescence program, it was tested whether overexpression of senescence factors or the putative transcriptional regulator NRF2, in the absence of other senescence stimuli, was sufficient to induce SLC25A51.

Overexpression of p16^{INK4A} and to a lesser degree p21^{Cip1/Waf1} and NRF2, was indeed sufficient to induce SLC25A51 protein (and other senescence markers) in MIN6 cells (FIG. 5A), while levels of another mitochondrial transporter, SLC25A38, remained unchanged. In addition, overexpression of SLC25A51, which led to a more than 3-fold increase in mitochondrial NAD⁺ levels, and a 2-fold increase in the mitochondrial NAD⁺/NADH ratio, but not its transport-dead K91A point mutant, or SLC25A38, was sufficient to induce p16^{INK4A}, p21^{Cip1/Waf1}, and γ-H2AX (FIGS. 5E-5G).

Thus, the data suggest that SLC25A51 upregulation downstream of p16^{INK4A}, possibly through its role in compartmentalizing NAD⁺ and controlling its redox balance, modulates senescence or could be part of a feed-forward loop that further drives the senescence program.

If metabolic changes downstream of SLC25A51's role in compartmentalizing NAD⁺ or controlling the cellular NAD⁺/NADH ratio contribute to the senescence program, it is likely that SLC25A51 depletion, in turn, could prevent senescence induction. Indeed, SLC25A51 depletion in MIN6 cells using stable shRNA transfection, which led to a reduction in mitochondrial NAD⁺, prevented H₂O₂-induced upregulation of senescence markers (p16^{INK4A}, p21^{Cip1/Waf1}, and γ-H2AX) (FIGS. 5B-5D). This finding aligns with a recent study in mammalian U2OS cells, where SLC25A51 knockdown prevented H₂O₂-induced DNA damage by increasing nuclear availability of NAD⁺ for PARP1-dependent DNA repair and phosphorylation of histone γ-H2AX, a marker of DNA damage and senescence.

Thus, SLC25A51 overexpression induces senescence markers in MIN6 cells.

### Example 8. In Vivo Studies of Inhibition of SLC25A51 in Insulin-Resistant Male Mice

Chronological aging and metabolic insults induce pancreatic beta-cell senescence and insulin resistance in mice and humans. The accumulation of senescent beta cells during aging contributes to insulin resistance and T2D through both cell-autonomous and non-autonomous mechanisms. Developing strategies to prevent beta-cell senescence could have major implications for the treatment of T2D. Given that SLC25A51 is a key regulator of mitochondrial NAD⁺ metabolism and its levels correlate with increased senescence and inflammation, it was investigated whether inhibiting SLC25A51 in insulin-resistant male mice could prevent age-associated diabetes.

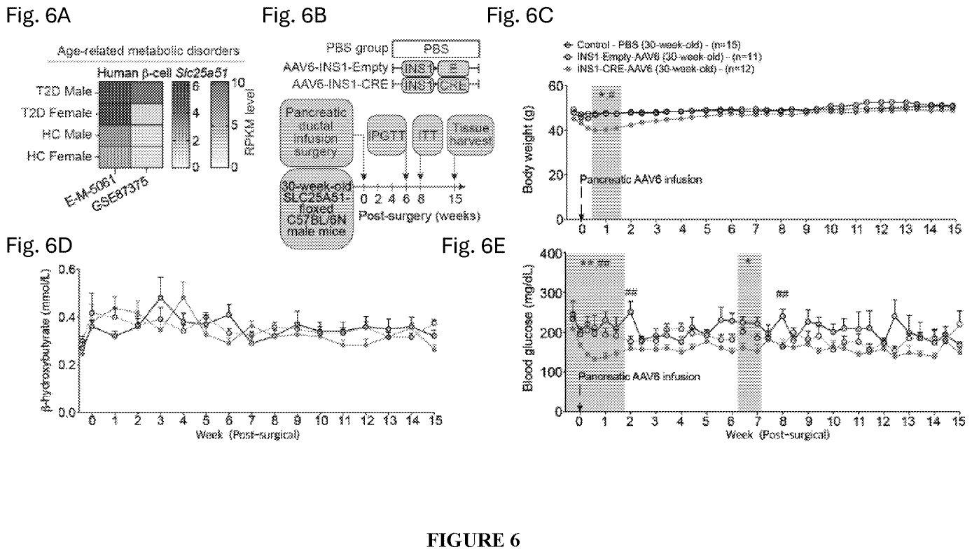

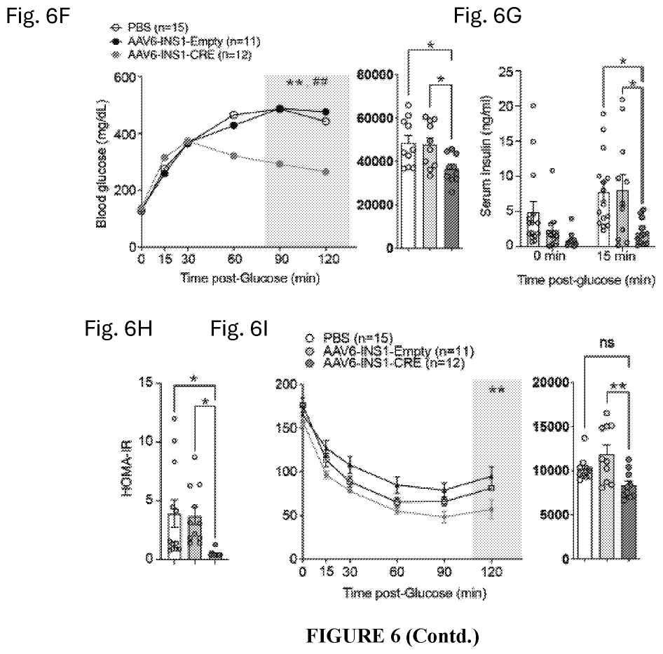

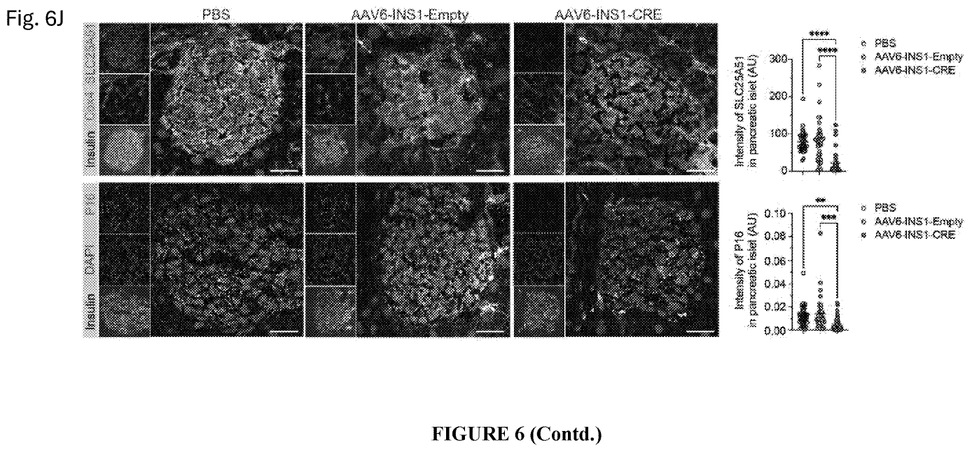

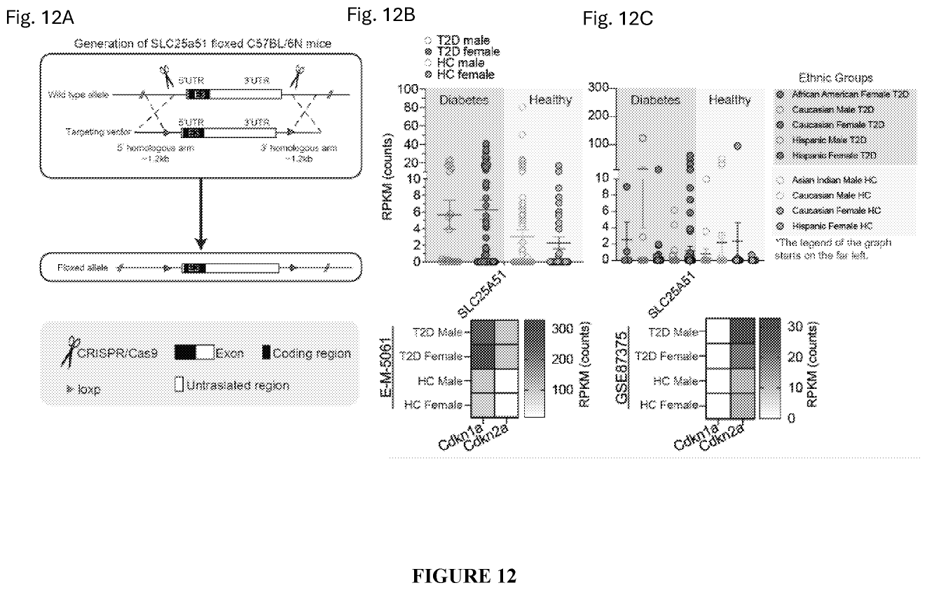

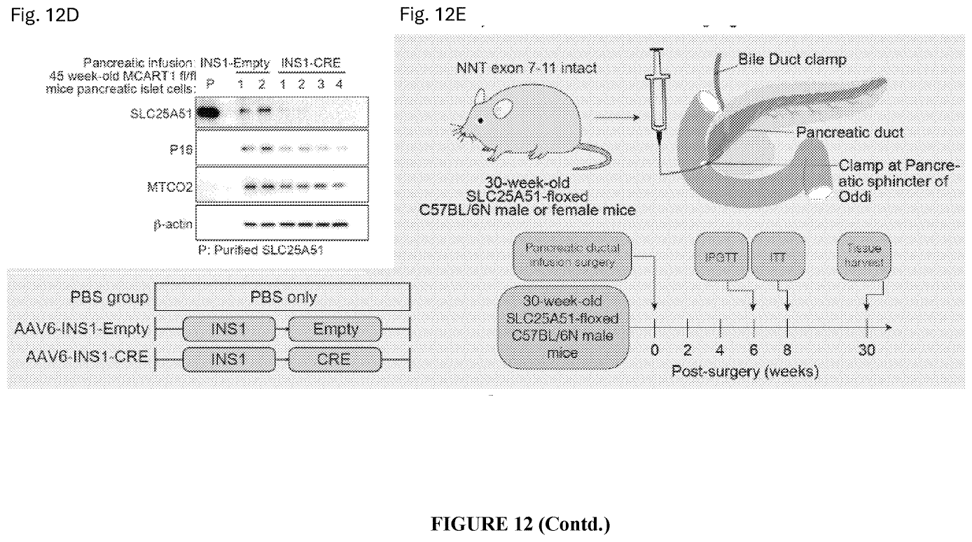

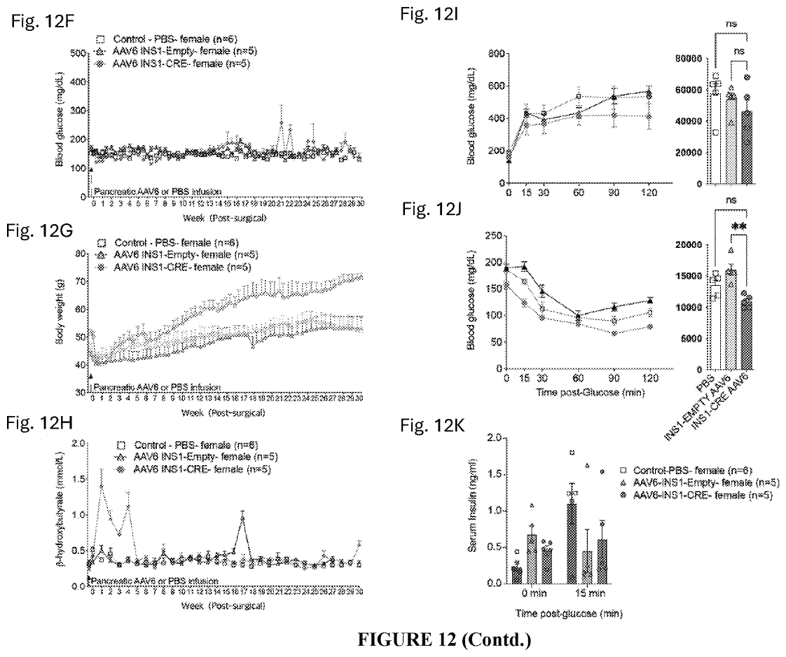

As in mice, SLC25A51 was predominantly expressed in beta cells in the human pancreas (FIG. 7B). To assess SLC25A51's potential as a therapeutic target, SLC25A51 expression was analyzed in scRNA-seq datasets from humans. SLC25A51 levels were higher in male T2D patients compared to healthy male controls from two different datasets, similar to the expression of Cdkn1a (p16^{INK4A}) and Cdkn2a (p21^{Cip1/Waf1}) (FIGS. 6A, 12B, 12C).

To test the effect of SLC25A51 deletion on glucose homeostasis and insulin sensitivity, SLC25A51 were specifically deleted in beta cells, employing a SLC25A51 floxed mouse model (FIGS. 6A, 12A, 12E). Using AAV6 viruses harboring Cre recombinase driven by the beta-cell-specific insulin 1 promoter (INS1-Cre), SLC25A51 was selectively deleted in pancreatic beta cells of 30-week-old male and female mice. Control mice received AAV6 viruses with an empty vector (INS1-Empty) or PBS via pancreatic duct injection (FIGS. 6A, 12A, 12E). Fifteen weeks post-surgery, male mice were sacrificed for pancreatic islet and tissue analysis. Western blot and IF staining confirmed the loss of SLC25A51 protein in the pancreatic islets of INS1-Cre-infused mice compared to INS1-Empty mice (FIGS. 61, 12D). Furthermore, beta-cell-specific SLC25A51 knockout (OKO) mice exhibited reduced p16^{INK4A} levels, indicating lower beta-cell senescence (FIGS. 6J, 12D).

Since age-related insulin resistance is observed in both mice and humans, whether the loss of SLC25A51 in pancreatic beta cells affected insulin sensitivity was further investigated. While infusion of INS1-CRE AAV6 viruses resulted in a slight reduction in body mass and blood glucose levels compared to the INS1-Empty AAV6 and PBS-infused mice (FIGS. 6B, 6D), circulating beta-hydroxybutyrate levels remained unchanged (FIG. 6C). Interestingly, blood glucose levels in SLC25A51 βKO mice remained lower after virus infusion, while body weight stabilized to pre-surgery levels (FIG. 6D).

To assess glucose clearance and insulin sensitivity, intraperitoneal glucose tolerance test (IPGTT) and insulin tolerance test (ITT) were performed. SLC25A51 βKO mice displayed improved glucose clearance in the IPGTT. Insulin measurements at 0 and 15 min during IPGTT and ITT revealed hyperinsulinemia in the two control groups, while SLC25A51 βKO mice exhibited lower systemic insulin levels (PBS:7.84±5.2 ng/ml; AAV6-INS1-empty:8.07±7.3 ng/ml; AAV6-INS1-CRE:2.07±1.8 ng/ml), likely explaining the improved insulin sensitivity (FIGS. 6E-6H). HOMA-IR analysis indicated insulin resistance in the PBS and AAV6-INS1-Empty groups, whereas the AAV6-INS1-CRE infused group was non-insulin-resistant (FIG. 6G). Like male mice, female mice also showed improved insulin sensitivity (FIG. 12J). However, glucose levels, serum insulin levels, and IPGTT showed minimal effects in female mice (FIGS. 12F-121, 12K). Furthermore, IF staining suggested that the improved metabolic phenotype in male mice upon beta-cell-specific SLC25A51 deletion was due to lowered levels of p16^{INK4A}-positive senescent beta cells (FIGS. 61, J).

Thus, the results indicate that beta cell-specific deletion of SLC25A51 improves age-associated glucose homeostasis and insulin resistance in mice.

### Example 9. In Vivo Studies of β-Cell-Specific SLC25A51 Knockout Mice

β-cell-specific SLC25A51 knockout male mice (INS2-KO) were generated. 30-week-old C57BL/6N wild-type (WT) and β-cell-specific SLC25A51 knockout male mice (INS2-KO) (n=5 per group) were sacrificed. Pancreatic islets were isolated and treated with or without Nutlin-3a (10 μg/mL, 6 h) or CDDO-Im (10 nM, 6 h) to activate p53 and Nrf2, respectively. Samples were then collected for qPCR analysis of SLC25A51 and CRE mRNA expression levels.

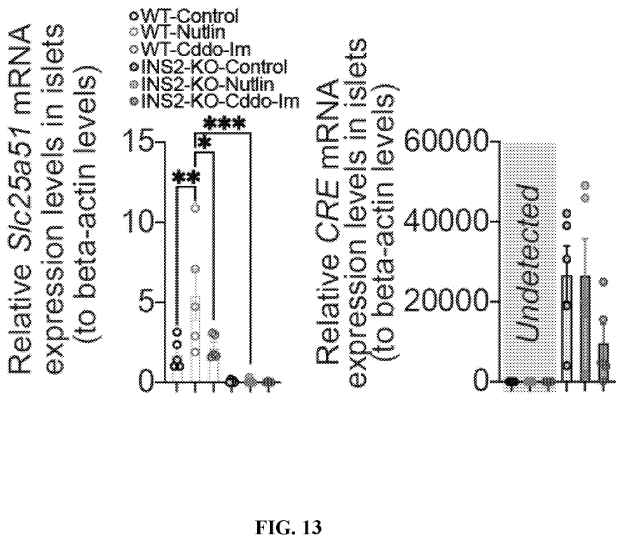

Following treatment with Nutlin-3A, expression levels of SLC25A51 were found to be significantly down-regulated in the INS2-KO mice when compared to wild-type mice. In contrast, CRE expression levels were upregulated upon treatment with Nutlin-3a, both in wild-type and INS2-KO mice (FIG. 13).

### Example 10. In Vivo Studies of Aging in β-Cell-Specific SLC25A51 Knockout Mice

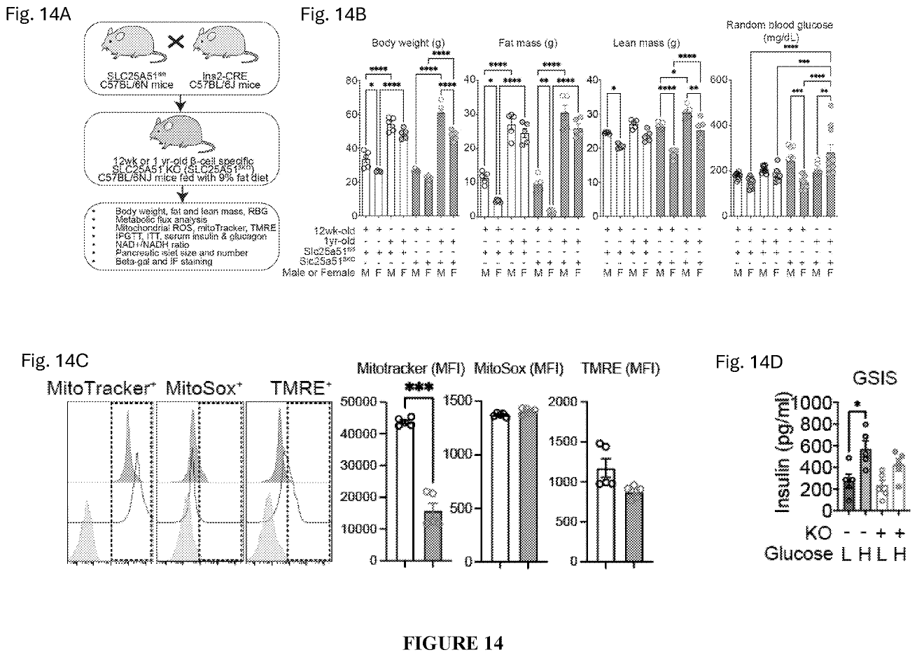

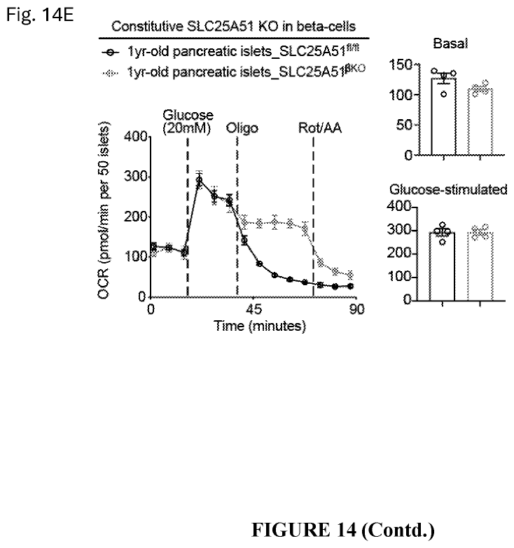

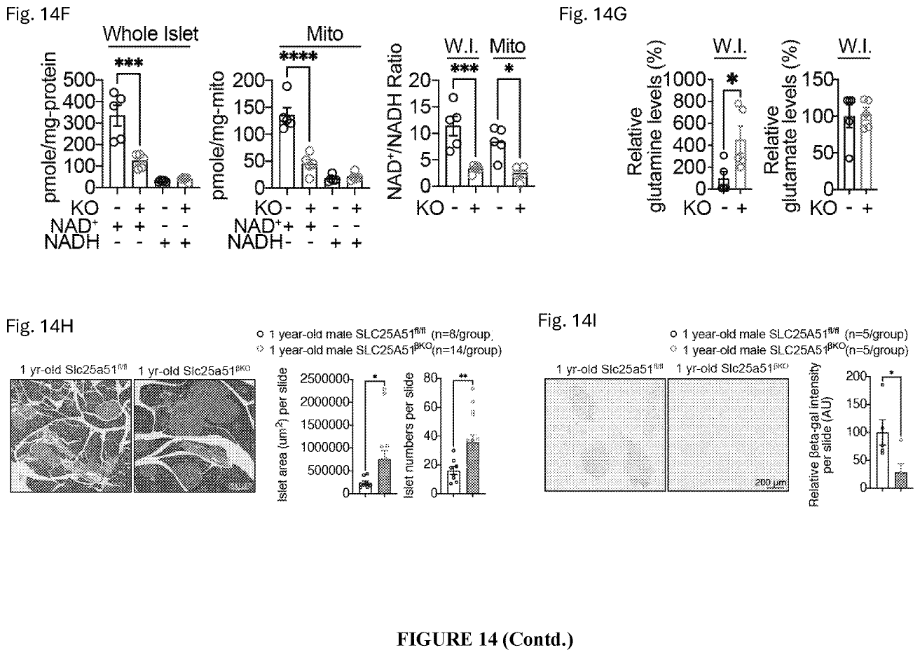

To study the effects of SLC25A51 inhibition during aging, 12-week-old and 1-year-old C57BL/6N wild-type (WT) and β-cell-specific SLC25A51 knockout male and female mice (Slc25a51^{βKO}) were generated and maintained on a 9% fat diet throughout life (FIG. 14A).

Measurements of body weight, fat mass, lean mass and random blood glucose levels indicated that body weight and fat mass increased with age in wild-type as well as Slc25a51^{βKO} mice, for both male and female mice. However, while random blood glucose levels showed differences among male and female mice, with the levels being significantly increased in female Slc25a51^{βKO} mice at 1 year compared to 12 weeks (FIG. 14B).

Pancreatic islets were isolated from 1-year-old WT and Slc25a51^{βKO} male mice and were stained with MitoTracker (mitochondria labeling), MitoSox (superoxide detector), and Tetramethylrhodamine ethyl ester (TMRE) (active mitochondria staining). Glucose-stimulated insulin secretion (GSIS) assays were performed on isolated pancreatic islets by sequential incubation in low (2.8 mM) and high (17.5 mM) glucose in Krebs Ringer Bicarbonate buffer. Supernatants were collected at each step, and insulin levels were quantified by ELISA using insulin standards. Quantification of the staining studies showed a significant decrease in the mean fluorescence intensity (MFI) by MitoTracker in wild-type mice compared to Slc25a51^{βKO} mice (FIG. 14C). GSIS studies showed a significant increase in insulin levels in wild-type mice when incubated with high glucose compared to low glucose (FIG. 14D).

Next, isolated pancreatic islets were evaluated for oxygen consumption rate (OCR) with a Seahorse extracellular flux analyzer XFe96 (Agilent). Freshly isolated pancreatic islets were first placed in each well with low glucose (2 mM) for 30 minutes in non-C02 incubator. After recording basal cellular respiration, cells were treated sequentially with glucose (20 mM), oligomycin (2 ug/ml), and rotenone/antimycin A (2 uM). Basal and glucose-stimulated OCR was compared between WT and Slc25a51^{βKO} male mice, and showed no significant differences between the two groups (FIG. 14E).

Whole pancreatic islets (WI) were isolated from each WT and Slc25a51^{βKO} male mouse, and mitochondria were pooled from five mice per sample to achieve a mitochondrial protein concentration of approximately 0.5 mg/mL. Whole-islet NAD⁺ and NADH levels were quantified using NAD+/NADH Glo kit (Promega) with NAD⁺/NADH standards and normalized to protein content determined by bovine serum albumin (BSA) standards. Mitochondrial NAD⁺ and NADH levels were measured in a similar manner, with normalization accounting for pooling from five mice per sample. After obtaining NAD+ and NADH levels, NAD+/NADH ratio was calculated. Glutamine and glutamate levels were also quantified using Glutamine/Glutamate Glo kit (Promega) with standards. Comparison of the results showed that while whole islet and mitochondrial NAD+ levels were decreased significantly in Slc25a51^{βKO} mice compared to wild-type mice, NADH levels showed no significant changes. As a result, whole islet and mitochondrial NAD⁺/NADH ratio was were decreased significantly in Slc25a51^{βKO} mice compared to wild-type mice (FIG. 14F). While relative glutamine levels were significantly increased in Slc25a51^{βKO} mice compared to wild-type mice, relative glutamate levels showed no significant difference (FIG. 14G).

Finally, pancreatic tissues were collected and either fixed in 4% paraformaldehyde (PFA) or snap-frozen in optimal cutting temperature (OCT) compound with 2-methylbutane. Fixed tissues were embedded in paraffin and processed for hematoxylin and eosin (H&E) staining, whereas snap-frozen tissues were subjected to beta-galactosidase staining. Quantification of microscopy images showed an increase in islet area as well as islet numbers in Slc25a51^{βKO} mice compared to wild-type mice, whereas beta-galactosidase staining revealed a decrease in relative beta-galactosidase intensity in Slc25a51^{βKO} mice compared to wild-type mice (FIGS. 14H and 14I).

Thus, the results demonstrate the role of SLC25A51 in the control of pancreatic islet function and indicate that beta cell-specific deletion of SLC25A51 improves pancreatic function in mice.

We claim:

1. A method for detecting the presence of cellular senescence in a subject, the method comprising:
(a) detecting the level of one or more biomarkers comprising solute carrier family 25 member 51 (SLC25A51) or nicotinamide adenine dinucleotide (NAD⁺) in a biological sample from the subject; and
(b) comparing the level of the one or more biomarkers with a control sample,
wherein an increase in the level of SLC25A51 in the subject as compared to the control sample indicates the presence of cellular senescence in the subject; and/or
wherein a decrease in the level of NAD+ in the subject as compared to the control sample indicates the presence of cellular senescence in the subject.

2. The method of claim 1, wherein the level of SLC25A51 is a level of the SLC25A51 mRNA or SLC25A51 protein.

3. The method of claim 1, further comprising determining the NAD⁺/NADH ratio in the sample from the subject, wherein a decrease in the NAD⁺/NADH ratio as compared to the control sample indicates the presence of cellular senescence in the subject.

4. The method of claim 1, further comprising detecting the level of one or more molecules selected from the group consisting of P16^{INK4A}, P21^{Cip1/Waf1}, γ-H2AX, NAMPT, glutamine, glutamate, cellular ammonia and serum secretory associated phenotype (SASP) factors.

5. The method of claim 1, wherein the control sample is an earlier-in-time biological sample obtained from the subject, or wherein the control sample is a biological sample obtained from a younger subject.

6. The method of claim 1, wherein the biological sample is obtained from blood, testis, breast, pancreas, or liver tissue of the subject.

7. The method of claim 6, wherein the biological sample is obtained from pancreas tissue and comprises pancreatic islet cells.

8. The method of claim 7, wherein the pancreatic islet cells comprise pancreatic β-cells.

9. A method of improving pancreatic β-cell function in a subject in need thereof, the method comprising administering to the subject a therapeutically effective amount of an SLC25A51 inhibitor, thereby improving pancreatic β-cell function in the subject.

10. The method of claim 9, wherein administration of the SLC25A51 inhibitor increases glucose clearance, increases insulin sensitivity, and/or decreases body weight in the subject compared to a control.

11. The method of claim 10, wherein glucose clearance is measured by a glucose tolerance test and insulin sensitivity is measured by an insulin tolerance test.

12. The method of claim 10, wherein the blood glucose level in the subject after administration of the SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control.

13. The method of claim 10, wherein the circulating insulin level in the subject after administration of SLC25A51 inhibitor is at least 1.1 fold, at least 1.5 fold, at least 2 fold, at least 5 fold, at least 10 fold, at least 20 fold, at least 50 fold, at least 100 fold, at least 200 fold lower compared to the control.

14. The method of claim 10, wherein the body weight of the subject after administration of the SLC25A51 inhibitor is at least 1.1 fold, at least 1.5 fold, at least 2 fold lower compared to the control.

15. The method of claim 9, wherein administration of the SLC25A51 inhibitor delays the onset of senescence in pancreatic β-cells of the subject.

16. The method of claim 15, wherein the expression levels of one or more genes selected from the group consisting of CDKN2A, CDKN1A, CXCL10, IL-1β, and IL-6 in a biological sample from the subject is at least 1.1 fold, at least 1.5 fold, at least 2 fold, at least 5 fold, at least 10 fold, at least 20 fold, at least 50 fold, at least 100 fold, at least 200 fold lower compared to a control.

17. The method of claim 15, wherein the levels of p16^{INK4A}-positive senescent beta cells in the subject is at least 1.1 fold, at least 1.5 fold, at least 2 fold, at least 5 fold, at least 10 fold, at least 20 fold, at least 50 fold, at least 100 fold, at least 200 fold lower compared to a control.

18. The method of claim 10, wherein the control is measured prior to administration of the SLC25A51 inhibitor to the subject.

19. The method of claim 9, wherein the SLC25A51 inhibitor is selected from the group consisting of a small molecule, an antibody, an antisense oligonucleotide, an siRNA, an shRNA, and a miRNA.

20. The method of claim 19, wherein the SLC25A51 inhibitor is fludarabine phosphate.

21. The method of claim 9, wherein the subject has diabetes.

22. The method of claim 9, wherein the subject is a human subject.

23. A method of treating a metabolic disorder in a subject, the method comprising administering to the subject a therapeutically effective amount of an SLC25A51 inhibitor, thereby treating the metabolic disorder in the subject.

24. The method of claim 23, wherein the metabolic disorder is associated with cellular senescence and is selected from the group consisting of diabetes, metabolic syndrome (MetS), metabolic cardiovascular diseases, osteoporosis, atherosclerosis, hepatic steatosis, osteoarthritis, idiopathic pulmonary fibrosis, obesity-induced anxiety, tau-mediated neurodegenerative disease and chronic kidney disease.

25. The method of claim 24, wherein the SLC25A51 inhibitor is selected from the group consisting of a small molecule, an antibody, an antisense oligonucleotide, an siRNA, an shRNA and a miRNA.

26. The method of claim 25, wherein the SLC25A51 inhibitor is fludarabine phosphate.

27. The method of claim 23, wherein the subject has diabetes.

28. The method of claim 23, wherein the subject is a human subject.
